(19) United States
(12) Patent Application Publication
(10) Pub. No.: US 2011/0044954 A1
(43) Pub. Date: Feb. 24, 2011

(54) Methods of producing germ-like cells and related therapies

(76) Inventors: Steven Stice, Athens, GA (US); Franklin West, Athens, GA (US)

Correspondence Address: Henry D. Coleman, 714 Colorado Avenue, Bridgeport, CT, 06605-1601, US

(21) Appl. No.: 12/583,402

(22) Filed: Aug. 20, 2009

(51) Int. Cl. A61K 35/12; C12N 5/071; C12N 5/07; C12N 5/00; A61P 15/00

(52) U.S. Cl. 424/93.7; 435/377; 435/366; 435/354; 435/325

(57) ABSTRACT

The present invention relates to methods of producing germ-like cells (GLCs) from embryonic stem cells and induced pluripotent stem cells, GLCs produced by such methods, gametes derived from such GLCs, pharmaceutical compositions and kits containing such GLCs, screens that use GLCs to identify agents useful in enhancing mammalian reproductive health, and methods of treatment that use GLCs to enhance mammalian reproductive health.

### RELATED APPLICATIONS

Not applicable.

### FIELD OF THE INVENTION

The present invention relates to methods of producing germ-like cells (GLCs) from embryonic stem cells and induced pluripotent stem cells, GLCs produced by such methods, gametes derived from such GLCs, pharmaceutical compositions and kits containing such GLCs, screens that use GLCs to identify agents useful in enhancing mammalian reproductive health, and methods of treatment that use GLCs to enhance mammalian reproductive health.

### BACKGROUND OF THE INVENTION

Citations for all references are found after the experimental section. Numerical citations (designated by “[ ]”) are listed in “Reference Collection 1” except for the numerical citations in Part 3 of the Experimental Section, which are listed in “Reference Collection 3”. Name citations for the Experimental Section, Part 2, are listed in “Reference Collection 2”.

Throughout this application, various publications are referenced. The disclosures of all of these publications and those references cited within those publications in their entireties are hereby incorporated by reference into this application in order to more fully describe the state of the art to which this invention pertains.

Infertility is a major problem in the United States, with 7.4% of married couples considered to be clinically infertile [1]. To aid these couples, assisted reproductive technologies have been developed, but they are ineffective in treating the most severe cases, where there is an absence of germ cell production [2]. Human embryonic stem cells (hESCs) offer the means to further understand intrinsic and extrinsic factors involved in early and late germ cell development and survival [3-5]. New developments and discoveries using hESCs should ultimately lead to novel fertility treatments.

Several genes and their products are expressed during germ cell development and are used to follow germ cell differentiation. POU5F1 (also known as Oct4), a transcription factor involved in stem cell pluripotency, is also expressed in primordial germ cells (PGCs) and is highly conserved among species [6]. As PGCs become linage-restricted during germ cell development, POU5F1 becomes exclusively expressed in germ cells and is not expressed in somatic cells [7]. In the mouse, Ifitm3 and DPPA3 are specifically expressed in PGCs [8, 9]. Unrestricted germ cells first express Ifitm3 during pregastrulation, followed by DPPA3 expression at the late primitive streak stage of development. In addition, DDX4 (also known as VASA) is a highly conserved, functionally important germ cell gene that is expressed exclusively in germ cells in numerous species, including Drosophila, Xenopus, mice, and humans [10-13]. DDX4 is an RNA helicase that in Drosophila has been shown to be important for germ cell plasm formation, but its role has not yet been fully elucidated in the mammalian system [11]. However, male DDX4 homozygous knockout mice are sterile [12], and previous studies have used DDX4 in human ESC to PGC differentiation [3, 14]. Male mouse germ cells enter a state of mitotic arrest at 13.5 days post coitum. This quiescent state is not reversed until puberty is reached; the mitotic process is then continued, and meiosis first takes place [15]. Synaptonemal complexes formed during homologous chromosome pairing in meiosis involve numerous proteins, such as SYCP3 [16, 17]. SYCP3 expression is specific to meiotic germ cells, and knockout of this gene results in meiotic disruption and sterility [15, 18]. After the pairing of homologous chromosomes, crossing over occurs, which is controlled by proteins such as MLH1 [19, 20] and is associated with the formation of chiasmata during a crossing-over event [19]. MLH1

knockout mice exhibit disrupted meiosis, which results in sterility in both female and male mice [20]. The expression of these genes in human germ cells has been noted, but their study has been limited. Further understanding of these genes and others through in vitro human germ cell models is therefore warranted.

In an attempt to address the need for an in vitro model to better understand these developmental processes, several groups have reported the differentiation of mouse embryonic stem cells (mESCs) and hESCs into germ-like cells. mESCs have produced both male and female germ-like cells with protein profiles consistent with germ cell development [4, 5]. Cells in advanced differentiation cultures have even proven to undergo meiosis and erasure of methylation in imprinting genes, two hallmarks of normal germ cell development [21]. Nayernia et al. successfully showed that mESC-derived germ cells can produce offspring [22]. Differentiated hESCs have also proven to have protein profiles consistent with normal germ cell development (i.e., DDX4+) and undergo early stages of meiosis [3, 14]. Unfortunately, both mESC and hESC germ cell differentiation culture systems have produced relatively mixed populations, with less than 35% DDX4-positive cells derived from hESCs, and coexpression of other germ cell markers, such as POU5F1, was not determined [3, 14]. Traditionally, isolation of stem cell populations based on only a single marker is not as robust as that based on two or more markers. Previous work has established that mouse embryonic fibroblasts feeders and basic fibroblast growth factor are essential for culturing primary germ cells [23-25]; however, their role has not been examined in the derivation of germ-like cells from hESCs.

Therefore, the need exists for methods which will reliably produce adequately-sized and relatively genetically homogeneous populations of germ-like cells from which functioning gametes can be derived.

### SUMMARY OF THE INVENTION

The present invention relates to novel methods of producing homogeneous populations of germ-like cells (GLCs), including methods of cloning a pure population of embryonic stem cells (ESC)-derived GLCs. In preferred embodiments, methods of the present invention combine adherent clonal cell culture propagation with selection for a GLC population that expresses both germ cell specific and pluripotency proteins, DDX4 and POU5F1 respectively.

In certain embodiments, the invention provides a novel adherent hESC to GLC differentiation culture system that is capable of producing a highly enriched GLC population wherein greater than about 69% of cells co-express the germ cell markers DDX4 and POU5F1 and wherein greater than about 90% of GLCs express the meiotic markers SYCP3 and MLH.

The adherent differentiation system of the present invention offers at least the following advantages over embryoid body (EB) systems:
1. Feeder cells have proven to be important in differentiating hESCs into GLCs as cultures lacking feeders demonstrate reduced GLC numbers. Adherent differentiation cultures optimize the contact between differentiating hESCs and feeders. West, F. D., et al., Enrichment and differentiation of human germ-like cells mediated by feeder cells and basic fibroblast growth factor signaling. Stem Cells, 2008. 26(11): p. 2768-76.
2. Adherent differentiation limits spontaneous differentiation signaling stimulated by EBs and aggregates allowing for more uniform and selective differentiation Nishikawa, S., L. M. Jakt, and T. Era, Embryonic stem-cell culture as a tool for developmental cell biology. Nat Rev Mol Cell Biol, 2007. 8(6): p. 502-7.
3. Cells are easily isolated in an adherent system as cells are only a few layers thick, while EBs and aggregates are cell masses that do not easily disaggregated without harsh treatments.
4. The adherent system also allows for homogeneous exposure to signaling factors that enhance germ cell differentiation, while EBs and aggregates only allow for the appropriate concentration of signaling factors to be seen by outer cells and decreasing concentrations seen by inner cells forming a gradient Kee, K., et al., Bone morphogenetic proteins induce germ cell differentiation from human embryonic stem cells. Stem Cells Dev, 2006. 15(6): p. 831-7.

Certain embodiments of the invention utilize feeders, which express factors that are important for in vivo germ cell development such as KIT ligand and basic fibroblast growth factor, a known mitogen and regulator of differentiation, to further enhance GLC development from hESCs. GLC (DDX4+ POU5F1+) enriched cultures expressed significantly (p<0.05) higher levels of the pre-migratory, post-migratory and meiotic germ cell genes relative to hESC cultures. These cultures have also demonstrated the ability to be continually cultured for 20+ passages, while maintaining the percentage of DDX4+POU5F1+ cells, germ cell gene expression and a stable karyotype.

Significantly, in certain embodiments, methods of the invention are able to produce a clonal population of GLCs wherein greater than 90% of cells are DDX4+ POU5F1+ and express the meiotic markers SYCP3 and MLH1 at high levels in cells that have undergone advanced differentiation. GLCs produced by these methods are primed for meiosis and represent an excellent system for exploring the role of compositions such as STRA8 in human germ cell development.

Accordingly, in one embodiment, the invention provides a method of producing germ-like cells (GLCs) by:

(a) differentiating cultured embryonic stem cells to germ-like cells in an adherent differentiation culture system comprising an effective amount a basic fibroblast growth factor, wherein the cultured embryonic stem cells are differentiated until at least about 10+% (until the cells are readily isolated), preferably about 50%, about 60% to about 90%, or about 65% to about 85%, or about 70% to about 80% of the cells express one or more germ cell markers; and
(b) optionally isolating and collecting the germ-like cells.

In preferred embodiments of GLC production methods of the invention, one or more and preferably all of the following applies:

(a) the embryonic stem cells are human embryonic stem cells (hESCs) and the adherent differentiation culture system comprises mouse embryonic fibroblast feeder cells;
(b) prior to differentiation, the hESCs are cultured in a culture medium comprising an effective amount of basic fibroblast growth factor and one or more components selected from the group consisting of knockout serum replacement, a non-essential amino acid, an antiobiotic, and β-mercaptoethanol;
(c) prior to differentiation and during culturing, the hESCs are passaged between 1 to 200 times;
(d) the cultured hESCs are differentiated until about 65% to about 85% of the cells express DDX4 and POU5F1 (these are GLC cells);
(e) about 85% to about 95% of the further differentiated cells express the meiotic markers SYCP3 and MLH1 (differentiated GLC); and
(f) the differentiated GLC can form haploid germ like cells (1 to 50%)

In one embodiment of a GLC production method of the invention, the adherent differentiation culture system comprises mouse embryonic fibroblast (MEF) feeder cells that have been transformed to express, express at an intermediate level, or not express KIT ligand (KITL).

In another embodiment of a GLC production method of the invention, cultured ESCs are differentiated in a medium comprising a member of the TGF-β family, most preferably BMP4. Alternatively, cultured ESCs are exposed to a member of the TGF-β family, most preferably BMP4, prior to differentiation.

In another embodiment of a GLC production method of the invention, the adherent differentiation culture system comprises the extracellular matrix of fibroblast feeder cells, preferably MEF extracellular matrix.

In another embodiment of a GLC production method of the invention, the invention provides a method of producing a pure population of germ-like cells, the method comprising:

(a) differentiating cultured embryonic stem cells to germ-like cells in an adherent differentiation culture system comprising a fibroblast growth factor, wherein the cultured embryonic stem cells are differentiated until at least about 10+% (so that the cells may be readily isolated), about 60% to about 90%, or about 65% to about 85%, or about 70% to about 80% of the cells express at least one germ cell marker;
(b) selecting individual differentiated cells that express at least one germ cell marker, propagating selected individual differentiated cells to form cell lines, and selecting for further differentiation those cell lines in which the cells (preferably, about 85% or more) express at least one germ cell marker; and
(e) differentiating selected cells to meiotic germ-like cells in an adherent differentiation culture system.

In another embodiment, GLC which are produced express markers including two, preferably three, four or five of acrosin, haprin, protamine 1, protamine 2 and RNF17.

To our knowledge, the above cloning method of the invention has yielded the highest proportion to date of germ-like cells derived from either mouse or human ESCs. This is an unexpected result.

In certain embodiments of the cloning methods of our invention, selected individual differentiated cells that express at least one germ cell marker are transduced with a reporter system containing STRA8 before being propagated to form cell lines, and after propagation, those cell lines which overexpress STRA8 and in which about 85% or more of the cells express at least one germ cell marker are selected for further differentiation.

Induced pluripotent stem cells (iPSCs) can also be differentiated into GLCs using the adherent differentiation culture system techniques of the invention described above.

In other embodiments, the invention provides GLCs made by the methods of the invention described above, as well as gametes derived from such GLCs, pharmaceutical compositions and kits containing such GLCs, screens that use GLCs to identify agents useful in enhancing mammalian reproductive health, and methods of treatment that use GLCs to enhance mammalian reproductive health.

In still other specific aspects, the present invention relates to a method for the in vitro production of sperm and oocyte from GLC and assisted reproductive techniques using GLC, including but not limited to in vitro fertilization, and intracytoplasmic sperm or germ cell injection.

These and other aspects of the invention are illustrated further in the Detailed Description of the Invention

### BRIEF DESCRIPTION OF THE FIGURES

FIG. 1 shows that expression of DDX4/POU5F1 protein is upregulated under enrichment conditions. hESCs were cultured on mouse embryonic fibroblast feeders (shown) or polyornithine- and laminin-coated plates in 20% knockout serum replacement media with or without bFGF (4 ng/ml) for 3, 10 (shown), or 30 days. DAPI (A) nuclear staining of hESCs (control, top row) exhibited colocalization with the pluripotency marker POU5F1 (C) and absence of the germ cell marker DDX4 (E). A merge of the three images are seen in (G). After 10 days of differentiation, the pluripotency marker POU5F1 (D) and germ cell marker DDX4 (F) displayed colocalization with DAPI (B), with similar results seen at day 30. DDX4 proved to have novel nucleolar localization ([F]; enlarged in the inset), and a merge of these images is shown in (H). Scale bars=10 μm, insets 5 μm. Abbreviations: bFGF, basic fibroblast growth factor; DAPI, 4,6-diamidino-2-phenylindole; hESC, human embryonic stem cell.

FIG. 2 shows enrichment conditions result in increased DDX4+ POU5F1+ cells, with bFGF playing a role under feeder-free conditions. Flow cytometry was used to quantify the DDX4_ POU5F1_ cell population ([A], day 10 with MEFS and bFGF shown). Flow analysis showed a significant (**, p less than 0.05; n=4) temporal effect, with an increase in DDX4+ POU5F1+ cells at day 10 compared with the hESC control ([B]; days 3, 10 and 30 with and without feeders). Under feeder-free conditions, bFGF (*, p less than 0.05) caused an increase in the percentage of DDX4+POU5F1+ at days 10 and 30.

Abbreviations: bFGF, basic fibroblast growth factor; hES, human embryonic stem; MEFS, mouse embryonic fibroblasts.

FIG. 3 shows feeder conditions resulted in higher gene expression of premigratory and migratory genes. hESCs were grown for 3, 10, and 30 days with and without feeders in the presence of 4 ng/ml of bFGF and were analyzed for gene expression by quantitative reverse transcription-polymerase chain reaction. The results showed that cells cultured on feeders exhibited significantly (**, p less than 0.05; n=4) higher expression of POU5F1, DAZL, and NANOG at days 10 and 30; of DDX4 at day 10; and of Ifitm3 and DPPA3 at day 30, relative to feeder-free conditions. Temporal effects were significant (*, p less than 0.05) under feeder culture conditions for DAZL and NANOG gene expression at days 10 and 30 relative to hESCs. POU5F1 and DDX4 were higher at day 10, and Ifitm3 was higher at day 30, relative to hESCs. Feeder-free cultures showed upregulation of DAZL at day 10, and all other genes (Ifitm3, POU5F1, DPPA3, NANOG, and DDX4) showed no significant increase relative to hESC controls. (Note scale differences.) Abbreviation: hESC, human embryonic stem cell.

FIG. 4 shows feeder conditions resulted in higher gene expression of genital ridge, spermatogonia, and meiotic-stage gene expression. hESCs were grown for 3, 10, and 30 days with and without feeders in the presence of 4 ng/ml of basic fibroblast growth factor and were analyzed for gene expression by quantitative reverse transcription-polymerase chain reaction. Feeder cell cultures resulted in significantly (**, p less than 0.05; n=4) increased expression of PIWIL2, PUM2, and MLH1 genes for days 10 and 30; of the DAZ1-4 cluster at day 10; and of NANOS1 and SYCP3 at day 30 relative to feeder-free conditions. Temporal effect under feeder culture conditions resulted in significant (*, p less than 0.05) upregulation of PIWIL2, PUM2, and NANOS1 at days 10 and 30 relative to hESCs and SYCP3 expression at day 30. Feeder-free cultures were upregulated for the genes NANOS1 and PIWIL2 at days 10 and 30 relative to hESCs; however, PUM2, DAZ1-4, SYCP3, and MLH1 showed no upregulation at day 10 or 30. (Note scale differences.) Abbreviation: hESC, human embryonic stem cell.

FIG. 5 shows expression of meiotic markers in differentiated cultures. hESCs were cultured on mouse embryonic fibroblast feeders in 20% knockout serum replacement medium with bFGF (4 ng/ml). Undifferentiated hESCs (control, top row) did not express the meiotic markers MLH1 ([A, B], merge with 4,6-diamidino-2-phenylindole [DAPI]) or SYCP3 ([C, D], merge with DAPI). After 16 days of differentiation, the meiotic markers MLH1 ([E, F], merge with DAPI) and SYCP3 ([G, H], merge with DAPI)

displayed colocalization with DAPI. This was not observed at day 10 or day 30.
Abbreviations: bFGF, basic fibroblast growth factor; hESC, human embryonic stem cell.

Figure X shows that flow cytometry analysis expression of DDX4/POU5F1 protein is upregulated under enrichment conditions when iPSC were cultured on mouse embryonic fibroblast feeders in 20% knockout serum replacement media with or without bFGF (4 ng/ml) for 10 days. After 10 days the percentage of DDX4/POU5F1 positive cells were increased over control iPSC. Additional culture for 16 days total increased the percentage of MLH1 SYCP3 in these conditions.

FIG. 6 shows differentiation of Germ-Like Cells from Human Embryonic Stem Cells. Immunocytochemistry indicated that POU5F1+ DDX4− (B and C-merge) hESCs were differentiated into DDX4+ POU5F1+ (F and G-merge) germ-like cells on feeders in 20% KSR media with media changes every other day and without passaging for 10 days. Human neural progenitor (hNPCs (J and K-merge)) and co-cultured feeder (N and O-merge) cells showed no expression of POU5F1 or DDX4. Analysis by flow cytometry indicated a small subpopulation of POU5F1+ DDX4+ cells in hESCs (D; negative control in red, hESCs in blue), while a substantial increase was seen in day 10 cells (H; negative control in red, day 10 cells in blue). DDX4 expression profiles of hESCs and day 10 cells are cells positive for both the human nuclear antigen and POU5F1. POU5F1 and DDX4 (L and P, respectively; negative control in red, hNPs or feeders in blue) expression was never observed in hNPCs or feeder cells.

FIG. 7 shows loss of KITL Expression in Differentiation Cultures Causes Decrease Germ Cell Gene Expression and DDX4+ POU5F1+ Cells. (A) hESCs were differentiated on KITL +/+, +/− or −/− feeders in 20% KSR media for 10 days. Differentiation of hESCs on KITL −/− feeders significantly decreased (p<0.05) DAZL, KIT, CXCR4, DDX4, MLH1 and SYCP3 gene expression relative to hESCs differentiated on KITL +/+ feeders. (B) Differentiation on KITL +/− and −/− feeders significantly decreased (p<0.05) DDX4+POU5F1+ cells relative to hESCs differentiated on KITL +/+ in a dose dependent manner (*=Statistically Significant from hESCs, #=Statistically Significant from KITL +/+ Treatment).

FIG. 8 shows BMP Increased Germ Cell Gene Expression and DDX4+ POU5F1+ Cells. (A) hESCs were differentiated in 20% KSR media in the presents of 100 ng/ml of noggin, 0 ng/ml (control), 10 ng/ml or 100 ng/ml of BMP4 for 10 days. Noggin significantly inhibited (p<0.05) expression of IFITM3, POU5F1, NANOG, PUM2 and MLH1, relative to hESCs (control). BMP4 significantly increased (p<0.05) DPPA3, POU5F1, KIT, NANOG, PUM2 and MLH1 expression relative to control. (B) 100 ng/ml of Noggin significantly decreased (p<0.05) the number of DDX4+ POU5F1+ cells, while 100 ng/ml of BMP4 had no effect when compared to control treatment. (s=Statistically Significant from hESCs, #=Statistically Significant from Control (0 ng/ml of BMP4 and 0 ng/ml Noggin)).

FIG. 9 shows differentiation on Feeder Extracellular Matrix Decreased Germ Cell Gene Expression, but Maintained DDX4+ POU5F1+ Cell Number. (A) hESCs were differentiated on feeder ECM and on feeders (control) in 20% KSR media for 10 days. Differentiation of hESCs on ECM significantly decreased (p<0.05) expression of germ cell genes IFITM3, DPPA3, KIT, NANOG, PUM2, MLH1 and SYCP3 (*=Statistically Significant from Control).

(B) There was no significant change in the percentage of DDX4+ POU5F1+ cells relative to Control, but for hESC the percentage of DDX4+ POU5F1+ cells was significantly lower (p<0.05) than in both the Control and ECM treatment groups (*=Statistically Significant from hESCs, #=Statistically Significant from Control; hESCs and D10 data are the same as FIG. 7).

FIG. 10 shows Meiotic Germ Cell Gene Expression. SYCP3 and MLH1 demonstrated increased gene expression at Days 10 and 30, relative to Day 3, indicating an increase in meiotic activity (A-B). A similar increasing trend was also seen in DDX4 and POU5F1 expression at Days 10 and 30 with respect to Day 3 (C-D). Unlike protein expression, bFGF did not seem to play a role in gene expression. Statistically significant day effect (p<0.05) indicated by Arabic lettering.

FIG. 11 shows Expression of Meiotic Markers in Differentiated Cultures. hESCs were differentiated on feeders for 10, 16 and 30 days in 20% KSR medium with bFGF (4 ng/ml). Undifferentiated hESCs (control, top row) did not express the meiotic markers MLH1 (A, merge (B) with 4,6-diamidino-2-phenylindole [DAPI]) or SYCP3 (C, merge (D) with DAPI). After 16 days of differentiation, the meiotic markers MLH1 (E, merge (F) with DAPI) and SYCP3 (G, merge (H) with DAPI) displayed co-localization with DAPI. This was not observed at day 10 or day 30.

FIG. 12 shows Continual Expansion and Transduction of Human GLCs. Flow cytometry indicated that hESCs differentiated for 10 days on feeders in 20% KSR media with bFGF had a significant (p<0.05) increase in the number of DDX4+ POU5F1+ cells from 4.0% to 18.7% (A). GLCs were then passaged every 4 days under differentiation conditions with analysis at passage 5 (58.0%), 10 (58.7%) and 20 (60.7%) showing that DDX4+/POU5F1+ cells not only were maintained but significantly (p<0.05) increased. Continually cultured GLCs were then trasduced using a GFP+ lentiviral system with >90% of cells demonstrating expression after 48 hrs (B, phase; C, GFP). These cells have maintained high levels of expression for 5 passages and are still being cultured. Statistical significance (p<0.05) indicated by Arabic lettering.

FIG. 13 shows Clonal Isolation and Meiotic Differentiation of Clonal GLCs. Single cell FACS sorting of GFP+GLCs into 96 well plates containing feeders and 20% KSR media plus 10 μM of Y-27632 resulted in 5 stable lines of potential GLCs. Flow cytometry confirmed clones 1-3 GLC identity with DDX4+ POU5F1+ expression in >90% of cells (A), while clones 4-5 were only POU5F1+ and believed to be hESCs (B). Clonal GLCs underwent meiotic differentiation for 0, 6 and 10 days and were analyzed by flow for SYCP3 and MLH1 expression. Clones 1-3 were positive for both SYCP3 (75.4%-88% positive, C) and MLH1 (80.6%-87.6% positive, E) at day 10 and negative at day 0 and 6 (data not shown). Clones 4 and 5 produced <4%+ cells for either marker at days 0, 6 (data not shown) and 10 (SYCP3-D; MLH1-F). All panels shown are representative samples.

FIG. 14 shows a TZV family STRA8 vector containing CMV promoter driven puromycin resistance and a tetracycline responsive element (TRE)-CMV promoter driven STRA8, as proposed for use in prophetic Example 12.

### DETAILED DESCRIPTION OF THE INVENTION

The following terms shall be used to describe the present invention:

Unless otherwise noted, the terms used herein are to be understood according to conventional usage by those of ordinary skill in the relevant art. In addition to the definitions of terms provided below, definitions of common terms in molecular biology may also be found in Rieger et al., 1991 Glossary of genetics: classical and molecular, 5th Ed., Berlin: Springer-Verlag; and in Current Protocols in Molecular Biology, F. M. Ausubel et al., Eds., Current Protocols, a joint venture between Greene Publishing Associates, Inc. and John Wiley & Sons, Inc., (1998 Supplement). It is to be understood that as used in the specification and in the claims, “a” or “an” can mean one or more, depending upon the context in which it is used. Thus, for example, reference to “a cell” can mean that at least one cell can be utilized.

The present invention may be understood more readily by reference to the following detailed description of the preferred embodiments of the invention and the Examples included herein. However, before the present compositions and methods are disclosed and described, it is to be understood that this invention is not limited to specific specific conditions, or specific methods, etc., as such may, of course, vary, and the numerous modifications and variations therein will be apparent to those skilled in the art.

Standard techniques for growing cells, separating cells, and where relevant, cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al., 1989 Molecular Cloning, Second Edition, Cold Spring Harbor Laboratory, Plainview, N.Y.; Maniatis et al., 1982 Molecular Cloning, Cold Spring Harbor Laboratory, Plainview, N.Y.; Wu (Ed.) 1993 Meth. Enzymol. 218, Part I; Wu (Ed.) 1979 Meth. Enzymol. 68; Wu et al., (Eds.) 1983 Meth. Enzymol. 100 and 101; Grossman and Moldave (Eds.) 1980 Meth. Enzymol. 65; Miller (ed.) 1972 Experiments in Molecular Genetics, Cold Spring Harbor Laboratory, Cold Spring Harbor, N.Y.; Old and Primrose, 1981 Principles of Gene Manipulation, University of California Press, Berkeley; Schleif and Wensink, 1982 Practical Methods in Molecular Biology; Glover (Ed.) 1985 DNA Cloning Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering Principles and Methods, Vols. 1-4, Plenum Press, New York. Abbreviations and nomenclature, where employed, are deemed standard in the field and commonly used in professional journals such as those cited herein.

“Adherent differentiation culture systems” include any culture in which cells in contact with a suitable growth medium are present, and can be viable or proliferate while adhered to a substrate. The substrate may contain extracellular matrix components such as collagen, fibronectin or other matrix protein (as otherwise described), which can increase adhesion properties of the cells and provide additional growth signals. Those of ordinary skill in the art know how to select adherent culture systems that use feeder cells or are feeder free and that are useful in the methods of the claimed invention. For example, as described further in the Examples, we have used a system in which germ-like cells were enriched in an adherent culture system by growing them on MEF feeders or polyornithine (20 μg/ml) and laminin (5 μg/ml)-coated (feeder-free) plates with or without 4 ng/ml bFGF. Multi-tray bioreactors can be used, as can vessels such as the HYPERflask® (Corning, N.Y.). Micro-beads and systems such as the CellCube (Biotechnology Techniques 9:10 (October 1995)) can also be employed.

In certain embodiments, methods of the invention comprise or contemplate plating the cells in an adherent culture. As used herein, the terms “plated” and “plating” refer to any process that allows a cell to be grown in adherent culture. As explained, the term “adherent culture” refers to a cell culture system whereby cells are cultured on a solid surface, which may in turn be coated with a solid substrate that may in turn be coated with another surface coat of a substrate, such as those listed below, or any other chemical or biological material that allows the cells to proliferate or be stabilized in culture. The cells may or may not tightly adhere to the solid surface or to the substrate. In one embodiment, the cells are plated on matrigel coated plates, which is preferred. The substrate for the adherent culture may comprise any one or combination of cell support such as polyornithine, laminin, poly-lysine, purified collagen, gelatin, extracellular matrix, fibronectin, tenascin, vitronectin, entactin, heparin sulfate proteoglycans, poly glycolytic acid (PGA), poly lactic acid (PLA), poly lactic-glycolic acid (PLGA) and feeder layers such as, but not limited to, primary fibroblasts or fibroblast cells lines. Furthermore, the substrate for the adherent culture may comprise the extracellular matrix laid down by a feeder layer, laid down by the pluripotent human cells or cell culture or laid down by the definitive endoderm cells or cell culture.

A “cell population” refers to a plurality or a collection of cells. A cell population can have greater or less numbers of particular cells than other cells present in the population.

A “cell culture” refers to maintenance or growth of one or more cells in vitro or ex vivo. Thus, a cell culture is one or more cells in a growth or culture medium of some kind. A “culture medium” or “growth medium” are used interchangeably herein to mean any substance or preparation used for sustaining or maintaining viability of cells, or growing cells.

“Culturing of embryonic stem cells” may entail 1, 2, 3, 4, 5, 6, 7, 8, 9, 10 or more cell doublings. Such expanded or proliferated cells, cultures, populations and compositions can include 100,000, 5000,000, 1,000,000, 2,000,000-5,000,000 or more Germ like Cells (GLC).

The term “passaged” is used to describe the process of splitting cells and transferring them to a new cell vial or plate for further growth/regrowth. The adherent cells according to the present invention may be passaged using enzymatic (e.g., trypsin, Accutase™ or collagenase) passage, manual passage (mechanical, with, example, a spatula or other soft mechanical utensil or device) and other non-enzymatic methods, such as cell dispersal buffer. In preferred aspects of the invention, cells are passaged as few times as possible (preferably, 10 times or fewer) before being further used/differentiated, although in certain aspects of the invention, grown cells may be passaged up to several hundred times as otherwise described herein.

“Germ cell markers” include, but are not limited to DDX4, POU5F1, IFITM3, DPPA3, NANOG, NANOS1, PIWIL2, DAZ1, DAZ2, DAZ3, DAZ4, SYCP3, SYCP1, MLH1,CXCR4, PUM2, DAZL, KIT.

“Reporter systems” are well-known to those of ordinary skill in the art and comprise vectors that can include a selection marker. A “selection marker” or equivalent means a gene that allows the selection of cells containing the gene. “Positive selection” refers to a process whereby only cells that contain the positive selection marker will survive upon exposure to the positive selection agent or be marked. For example, drug resistance is a common positive selection marker; cells containing the positive selection marker will survive in culture medium containing the selection drug, and those which do not contain the resistance gene will die. Suitable drug resistance genes are neo, which confers resistance to G418, or hygr, which confers resistance to hygromycin, and puro which confers resistance to puromycin, among others. Other positive selection marker genes include genes that allow the identification or screening of cells. These genes can encode fluorescent proteins, lacZ, the alkaline phosphatase, and surface markers such CD8, among others. “Negative selection” refers to a process whereby cells containing a negative selection marker are killed upon exposure to an appropriate negative selection agent which kills cells containing the negative selection marker. For example, cells which contain the herpes simplex virus-thymidine kinase (HSV-tk) gene are sensitive to the drug gancyclovir (GANC). Similarly, the gpt gene renders cells sensitive to 6-thioxanthine. GFP is a preferred selection marker. A preferred reporter system comprises a viral vector comprising a first antibiotic resistance element under viral promoter control and a second antibiotic responsive-STA8 element under viral promoter control.

As used herein, the term “effective amount” refers to that amount or concentration of any component or material and where relevant, for an appropriate length of time which is used to produce an intended result in the present invention.

As used herein, the term “express” refers to the transcription of a polynucleotide or translation of a polypeptide in a cell, such that levels of the molecule are measurably higher in a cell that expresses the molecule than they are in a cell that does not express the molecule. Methods to measure the expression of a molecule are well known to those of ordinary skill in the art, and include without limitation, Northern blotting, RT-PCT, in situ hybridization, Western blotting, and immunostaining.

As used herein, the term “contacting” (i.e., contacting a definitive endoderm cell, with a compound) is intended to include incubating the compound and the cell together in vitro (e.g., adding the compound to cells in culture).

As used herein, the term “differentiate” refers to the production of a cell type that is more differentiated than the cell type from which it is derived. The term therefore encompasses cell types that are partially and terminally differentiated. As used herein, the term, “differentiated”, “differentiating”, “cell differentiation environment”, etc. refer to a cell culture condition (e.g. generally, a basal cell media) wherein the pluripotent cells are induced to differentiate, or are induced to become a human cell culture enriched in differentiated cells. Preferably, the differentiated cell lineage induced by the growth factor will be homogeneous in nature.

In certain embodiments of the present invention, the term “enriched” refers to a cell culture that contains more than approximately 50%, 55%, 60%, 65%, 70%, 75%, 80%, 85%, 90%, or 95% of the desired cell lineage, depending upon the type of cells and methods used to provide same.

The term “homogeneous,” refers to a population that contains more than approximately 50%, 60%, 70%, 80%, 85%, 86%, 87%, 88%, 89%, 90%, 91%, 92%, 93%, 94%, 95%, 96%, 97%, 98%, or 99% of the desired cell lineage. A homogeneous lineage may be obtained directly from the differentiation process without further purification of the cells or alternatively, flow cytometry and other techniques may be used to purify the cells.

Cells which are produced according to the present invention have several uses in various fields of research and development including but not limited to drug discovery, drug development and testing, toxicology, production of cells for therapeutic purposes and for transplantation as well as basic science research. These cell types express molecules that are of interest in a wide range of research fields. These include the molecules known to be required for the functioning of the various cell types as described in standard reference texts. These molecules include, but are not limited to, cytokines, growth factors, cytokine receptors, extracellular matrix, transcription factors, secreted polypeptides (hormones) and other molecules, and growth factor receptors.

“Embryonic stem cells (ESCs)” are stem cells derived from the inner cell mass of an early stage embryo known as the blastocyst. Embryonic Stem (ES) cells are pluripotent. This means they are able to differentiate into all derivatives of the three primary germ layers: ectoderm, endoderm, and mesoderm. As used herein, the term “pluripotent human cell” or “human embryonic stem cells” encompasses pluripotent cells obtained from human embryos, fetuses or adult tissues. In one preferred embodiment, the pluripotent human cell is a human pluripotent embryonic stem cell (hESC). In another embodiment the pluripotent human cell is a human pluripotent fetal stem cell, such as a primordial germ cell. In another embodiment the pluripotent human cell is a human pluripotent adult stem cell. As used herein, the term “pluripotent” refers to a cell capable of at least developing into one of ectodermal, endodermal and mesodermal cells. As used herein the term “pluripotent” refers to cells that are totipotent and multipotent. As used herein, the term “totipotent cell” refers to a cell capable of developing into all lineages of cells. The term “multipotent” refers to a cell that is not terminally differentiated. As also used herein, the term “multipotent” refers to a cell that, without manipulation (i.e., nuclear transfer or dedifferentiation inducement), is incapable of forming differentiated cell types derived from all three germ layers (mesoderm, ectoderm and endoderm), or in other words, is a cell that is partially differentiated. The pluripotent human cell can be selected from the group consisting of a human embryonic stem (ES) cell; a human inner cell mass (ICM)/epiblast cell; a human primitive ectoderm cell, such as an early primitive ectoderm cell (EPL); a human primordial germ (EG) cell; and a human teratocarcinoma (EC) cell. The human pluripotent cells of the present invention can be derived using any method known to those of skill in the art. For example, the human pluripotent cells can be produced using de-differentiation and nuclear transfer methods. Additionally, the human ICM/epiblast cell or the primitive ectoderm cell used in the present invention can be derived in vivo or in vitro. EPL cells may be generated in adherent culture or as cell aggregates in suspension culture, as described in WO 99/53021. Furthermore, the human pluripotent cells can be passaged using any method known to those of skill in the art, including, manual passaging methods, and bulk passaging methods such as antibody selection and protease passaging.

“Induced pluripotent stems cells” (iPSCs) are a type of pluripotent stem cell artificially derived from a non-pluripotent cell, typically an adult somatic cell, by inducing a “forced” expression of certain genes. iPS cells are typically derived by transfection of certain stem cell-associated genes into non-pluripotent cells, such as adult fibroblasts. Transfection is typically achieved through viral vectors, such as retroviruses. Transfected genes include the master transcriptional regulators Oct-3/4 (Pouf51) and Sox2, although it is suggested that other genes enhance the efficiency of induction. After 3-4 weeks, small numbers of transfected cells begin to become morphologically and biochemically similar to pluripotent stem cells, and are typically isolated through morphological selection, doubling time, or through a reporter gene and antibiotic selection. In methods of the invention, human iPSCs are preferred, and induced pluripotent stem cells derived from IMR90 lung fibroblast cells are most preferred. In other methods, GLCs could be produced from a subject and further differentiated into sperm and/or eggs which may have significant agricultural, veterinary and/or animal husbandry benefits, as well as other benefits in humans.

The terms “culture medium”, “cell medium” or “cell media” are used to describe a cellular growth medium in which embryonic stem cells and/or GLCs are grown or differentiated. Cellular media are well known in the art and comprise at least a minimum essential medium plus optional agents such as growth factors, including fibroblast growth factor, preferably basic fibroblast growth factor (bFGF), glucose, non-essential amino acids, glutamine, insulin, transferrin, beta mercaptoethanol, and other agents well known in the art. Preferred media include commercially available media such as DMEM/F12 (1:1) or alpha MEM media, each of which may be supplemented with any one or more of L-glutamine, knockout seum replacement (KSR), fetal bovine serum (FBS), non-essential amino acids, beta-mercaptoethanol, basic fibroblast growth factor (bFGF) and an antibiotic. Cell media useful in the present invention are commercially available and can be supplemented with commercially available components, available from Invitrogen Corp. (GIBCO) and Biological Industries, HyClone (Thermo Scientific), among numerous other commercial sources. One of ordinary skill in the art will be able to readily modify the cell media to culture ESCs and differentiate GSCs pursuant to the present invention. The Materials and Methods sections of the Examples presented hereinafter illustrate preferred culture media.

During differentiation, a cell culture condition (e.g. generally, a basal cell media) exists wherein the pluripotent cells are induced to differentiate, or are induced to become a human cell culture enriched in differentiated cells. Preferably, the differentiated cell lineage induced by the growth factor will be homogeneous in nature. The term “homogeneous,” refers to a population that contains more than approximately 50%, 60%, 70%, 80%, 85%, 86%, 87%, 88%, 89%, 90%, 91%, 92%, 93%, 94%, 95%, 96%, 97%, 98%, or 99% of the desired cell lineage. A homogeneous lineage may be obtained directly from the differentiation process without further purification of the cells or alternatively, flow cytometry and other techniques may be used to purify the cells, especially the pancreatic endoderm cells or liver endoderm cells.

A cell differentiation medium may contain a variety of components as described herein, including, for example, KODMEM medium (Knockout Dulbecco's Modified Eagle's Medium), DMEM, Ham's F12 medium (especially DMEM/F12 50:50), FBS or FCS (fetal bovine serum or fetal calf serum), Retinoic acid (RA) fibroblast growth factor, including FGF2 (fibroblast growth factor 2), FGF 8, FGF 10 (especially for pancreatic or liver endoderm cells), KSR, bone morphogenetic protein (BMP) 4, BMP8, BMP7, BMP2 or hLIF (human leukemia inhibitory factor). The cell differentiation medium can also contain supplements such as L-Glutamine, NEAA (non-essential amino acids), P/S (penicillin/streptomycin), and β-mercaptoethanol (β-ME). It is contemplated that additional factors may be added to the cell differentiation environment, including, but not limited to, fibronectin, laminin or other cell support, heparin, heparin sulfate, retinoic acid, members of the epidermal growth factor family (EGFs), members of the fibroblast growth factor family (FGFs) including FGF2, FGF8 and/or FGF10, members of the platelet derived growth factor family (PDGFs), transforming growth factor (TGF)/bone morphogenetic protein (BMP)/growth and differentiation factor (GDF) factor family antagonists including but not limited to noggin, follistatin, chordin, gremlin, cerberus/DAN family proteins, ventropin, high dose activin, and amnionless. TGF/BMP/GDF antagonists could also be added in the form of TGF/BMP/GDF receptor-Fc chimeras. Other factors that may be added include molecules that can activate or inactivate signaling through Notch receptor family, including but not limited to proteins of the Delta-like and Jagged families as well as inhibitors of Notch processing or cleavage. Other growth factors may include members of the insulin like growth factor family (IGF), insulin, the wingless related (WNT) factor family, and the hedgehog factor family. Additional factors may be added to promote definitive endoderm stem/progenitor proliferation and survival as well as survival and differentiation of derivatives of these progenitors.

The methods of the present invention contemplate that cells may be cultured with a feeder cell or feeder layer. The term “feeder cell” is used to describe a cell that is co-cultured with a target cell and stabilizes (in some cases it both enhances and stabilizes) the target cell in its current state of differentiation. A feeder layer comprises more than one feeder cell in culture. In one embodiment of the above method, conditioned medium is obtained from a feeder cell that stabilizes the target cell in its current state of differentiation. Any and all factors produced by a feeder cell that allow a target cell to be stabilized in its current state of differentiation can be isolated and characterized using methods routine to those of skill in the art. These factors may be used in lieu of a feeder layer, or may be used to supplement a feeder layer. Preferred feeder cells include mouse embryonic fibroblast (MEF) feeder cells, feeder cells derived from human embryonic stem cells, feeder cells derived from the spontaneous differentiation of human embryonic stem cells, feeder cells obtained from human placenta, feeder cells derived from human foreskin, and feeder cells from human postnatal foreskin fibroblasts. MEF feeder cells are particularly preferred.

As used herein, the term “stabilize” refers to the differentiation state of a cell. When a cell or cell population is stabilized, it will continue to proliferate over multiple passages in culture, and preferably indefinitely in culture; additionally, each cell in the culture is preferably of the same differentiation state, and when the cells divide, typically yield cells of the same cell type or yield cells of the same differentiation state. Preferably, a stabilized cell or cell population does not further differentiate or de-differentiate if the cell culture conditions are not altered, and the cells continue to be passaged and are not overgrown. Preferably the cell that is stabilized is capable of proliferation in the stable state indefinitely, or for at least more than 2 passages. Preferably, it is stable for more than 5 passages, more than 10 passages, more than 15 passages, more than 20 passages, more than 25 passages, or most preferably, it is stable for more than 30 passages. In certain embodiments, the cell is stable for greater than 1 year of continuous passaging.

In one embodiment, stem cells (pluripotent cells) to be differentiated into definitive GLCs are maintained in culture in a pluripotent state by routine passage until it is desired that they be differentiated into definitive GLCs. In some embodiments, a member of the TGF-β family is administered to the pluripotent cell. As used herein, the term “member of the TGF-β family” refers to growth factors that are generally characterized by one of skill in the art as belonging to the TGF-β family, either due to homology with known members of the TGF-β family, or due to similarity in function with known members of the TGF-β family. In certain embodiments, the member of the TGF-β family is selected from the group consisting of Nodal, Activin A, Activin B, TGF-β, BMP2 and BMP4. Additionally, the growth factor Wnt3a is useful for the production of definitive GLCs. In certain embodiments of the present invention, combinations of any of the above-mentioned growth factors can be used. It is not necessary that these components be added to the cells simultaneously.

With respect to some of the embodiments of differentiation methods described herein, the above-mentioned growth factors are provided to the cells so that the growth factors are present in the cultures at concentrations sufficient to promote differentiation of at least a portion of the stem cells to GLCs. In some embodiments of the present invention, the above-mentioned growth factors are present in the cell culture at a concentration of at least about 0.5 ng/ml, at least 1 ng/ml, at least 10 ng/ml, at least about 25 ng/ml, at least about 50 ng/ml, at least about 75 ng/ml, at least about 100 ng/ml, at least about 200 ng/ml, at least about 300 ng/ml, at least about 400 ng/ml, at least about 500 ng/ml, or at least about 1000 ng/ml.

In certain embodiments of the present invention, the above-mentioned growth factors are removed from the cell culture subsequent to their addition. For example, the growth factors can be removed within about one day, about two days, about three days, about four days, about five days, about six days, about seven days, about eight days, about nine days or about ten days after their addition. In a preferred embodiment, the growth factors are removed about four days after their addition.

Cultures of GLCs can be grown in medium containing reduced serum or no serum. In certain embodiments of the present invention, serum concentrations can range from about 0.1% to about 30% (v/v). In some embodiments, GLCs are grown with serum replacement. In preferred embodiments, both pancreatic endoderm cells and liver endoderm cells are preferably grown in basal cell media comprising about 1% to about 20% (vol.) fetal calf serum, more preferably about 10% fetal calf serum.

The progression of the hESC culture to GLCs can be monitored by quantitating expression of marker genes characteristic of these cells as well as the lack of expression of marker genes characteristic of hESCs, definitive GLCs and other cell types. One method of quantitating gene expression of such marker genes is through the use of quantitative PCR (Q-PCR). Methods of performing Q-PCR are well known in the art. Other methods which are known in the art can also be used to quantitate marker gene expression. Marker gene expression can be detected by using antibodies specific for the marker gene of interest.

Using the methods described herein, compositions comprising GLCs which are substantially free of other cell types can be produced. Alternatively, compositions comprising mixtures of hESCs and GLCs can be produced. For example, compositions comprising at least 5 GLCs for every 95 hESCs can be produced, or 5 GLCs for every 95 definitive endoderm cells can be produced. In still other embodiments, compositions comprising at least 95 GLCs for every 5 hESCs, or up to 80 or more GLCs for every 5 definitive endoderm cells can be produced. Additionally, compositions comprising other ratios of GLCs to hESCs are contemplated.

In some embodiments of the present invention, GLCs can be isolated by using an affinity tag, such as SSEA1, CXCR4 and KIT, that is specific for such cells. One example of an affinity tag specific for GLCs is an antibody that is specific to a marker polypeptide that is present on the cell surface of the GLCs desired to be purified but which is not substantially present on other cell types that would be found in a cell culture produced by the methods described herein.

It is contemplated that the pluripotent cells or GLCs which are used as starting materials can be dissociated to an essentially single cell culture. As used herein, an “essentially single cell culture” is a cell culture wherein during passaging, the cells desired to be grown are dissociated from one another, such that the majority of the cells are single cells, or two cells that remain associated (doublets). Preferably, greater than 40%, 50%, 60%, 70%, 80%, 90%, 95%, 96%, 97%, 98%, 99% or more of the cells desired to be cultured are singlets or doublets. The term encompasses the use of any method known now or later developed that is capable of producing an essentially single cell culture. Non-limiting examples of such methods include the use of a cell dispersal buffer, and the use of proteases such as trypsin, collagenase, dispase, and accutase. These proteases and combinations of certain of the proteases are commercially available. The invention contemplates that the cell culture can be dissociated to an essentially single cell culture at any point during passaging, and it is not necessary that the dissociation occur during the passage immediately prior to contact with the inhibitor. The dissociation can occur during one or more passages. Alternatively, the samples may be centrifuged to dissociate the cell culture.

The cells produced using the methods of the present invention have a variety of uses. In particular, the cells can be used as a source of nuclear material for nuclear transfer techniques and used to produce cells, tissues or components of organs for transplant. For example, gametes may be derived from GLCs produced in accordance with the invention and such gametes could be used in vitro or in vivo fertilization techniques. In addition, the cells may be used for toxicity or drug screens. When induced pluripotent stem cells (iPSC) are used to generate GLC, the GLC generated from cells taken from one individual and used to make make sperm and or eggs to treat infertility. Additionally, iPSC from a male could be used to generate GLC and in this case sperm and eggs from the same individual (ie prized bulls) could then be mated one bull produced eggs and one bull produces the sperm, or sperm and eggs from that one individual

In a preferred embodiment of the invention, germ-like cells (GLCs) are produced by first culturing human embryonic stem cells (hESCs) on MEF feeder cells in a medium comprising basic fibroblast growth factor (bFGF), knockout serum replacement (KSR), a non-essential amino acid, an antiobiotic, and mercaptoethanol. The cells are passaged between 2 to 300, or between 50 to 250, or between 100 to 200 times during culturing. Cells are passaged by mechanical dissociation and are replated on fresh feeders to prevent undirected differentiation, with daily medium changes. During culturing, cells are maintained in about 5% CO₂ and at a temperature of about 37° C.

Next, cultured ESCs are enriched and differentiated by growing them, for between 3 to 30, or 5 to 25, or 10 to 20, or 12 to 15 days on MEF feeder cells in a culture medium comprising a bFGF and knockout serum replacement (KSR). During enrichment and differentiation, cells are not passaged and are maintained in about 5% CO₂ and at a temperature of about 37° C. After differentiation, between about 60% to about 90%, or about 65% to about 85%, or about 70% to about 80% of the cells in the culture medium are DDX4-positive and POU5F1-positive cells. Preferably, after differentiation, between about 75% to about 99%, or 80% to 97%, or most preferably 85% to about 95% of the differentiated cells express the meiotic markers SYCP3 and MLH1.

The enriched and differentiated cells can then be cloned to produce a pure population of germ-like cells (GLCs) as follows. Individual differentiated cells that express at least one germ cell marker (preferably that express both DDX4 and POU5F1) are selected, are preferably transduced with a reporter system containing STRA8, and are propagated to form cell lines. Those cell lines that overexpress STRA8 and in which about 85% or more of the cells express at least one germ cell marker (preferably that express both DDX4 and POU5F1) are selected for further differentiation. Selected cells are then differentiated to meiotic germ-like cells in an adherent differentiation culture system. Preferably, cells selected for differentiation to meiotic germ-like cells also express both SYCP3 and MLH1. These techniques are further illustrated in Examples 10 and 11.

Mouse developmental studies have provided significant insight into the genes involved in meiosis with several reports indicating that expression of the retinoic acid 8 (Stra8) gene is essential for meiotic induction in mouse germ cells Koubova, J., et al., Retinoic acid regulates sex-specific timing of meiotic initiation in mice. Proc Natl Acad Sci USA, 2006. 103(8): p. 2474-9 (“Koubova”); Anderson, E. L., et al., Stra8 and its inducer, retinoic acid, regulate meiotic initiation in both spermatogenesis and oogenesis in mice. Proc Natl Acad Sci USA, 2008. 105(39) (“Anderson”): p. 14976-80; Baltus, A. E., et al., In germ cells of mouse embryonic ovaries, the decision to enter meiosis precedes premeiotic DNA replication. Nat Genet, 2006. 38(12): p. 1430-4. Stra8 expression was shown to be associated with meiotic entry and sex specific timing. Koubova; Bowles, J., et al., Retinoid signaling determines germ cell fate in mice. Science, 2006. 312(5773): p. 596-600.]. Later studies have suggested an essential role for Stra8 in meiotic initiation since Stra8 deficient mice present abnormal meiotic progression in cohesion, synapsis and recombination processes. Anderson (male); Baltus, A. E., et al., In germ cells of mouse embryonic ovaries, the decision to enter meiosis precedes premeiotic DNA replication. Nat Genet, 2006. 38(12): p. 1430-4.], (female). However functional translational germ cell studies on the role of Stra8 in human germ development are nonexistent because of a lack of appropriate and available experimental material and processes to probe the role of STRA8 in human germ cell meiosis.

Those of ordinary skill in the art will recognize that the above illustrative method can be varied using media, constructs, and apparatus that are well-known in the art. For example, viral vectors, markers, and cell media can be changed or adapted pursuant to the description provided herein and the knowledge of those of ordinary skill in the art.

Pharmaceutical compositions comprising effective amounts of GLCs are also contemplated by the present invention. These compositions comprise an effective number of GLCs, optionally in combination with a pharmaceutically acceptable carrier, additive or excipient. In certain aspects of the present invention, cells are administered to the patient in sterile saline. In other aspects of the present invention, the cells are administered in Hanks Balanced Salt Solution (HBSS) or Isolyte S, pH 7.4. Other approaches may also be used, including the use of cellular media as otherwise described herein, preferably in the absence of growth facts. Such compositions, therefore, comprise effective amounts or numbers of GLCs in sterile saline. These may be obtained directly by using fresh or cryopreserved cells.

Pharmaceutical compositions according to the present invention preferably comprise an effective number within the range of about 1.0×10² GLCs to about 5.0×10⁷ mononuclear cells, more preferably about 1×10⁴ to about 9×10⁶ cells, even more preferably about 1×10⁶ to about 8×10⁶ cells generally in solution, optionally in combination with a pharmaceutically acceptable carrier, additive or excipient. Effective numbers of GLCs, either within a sample of other cells or preferably, as concentrated or isolated cells, may range from as few as several hundred or fewer to several million or more, preferably at least about one thousand cells within this range. In aspects of the present invention whereby the cells are injected in proximity to the brain or spinal cord tissue to be treated, the number of cells may be reduced as compared to aspects of the present invention which rely on parenteral administration (including intravenous and/or intraarterial administration).

In using compositions according to the present invention, fresh or cryopreserved GLCs (prepared using methods well known in the art from fresh GLCs) may be used without treatment with a differentiation agent or GLCs may be used with or without an effective amount of a differentiation agent prior to being used in a method of treatment as described herein.

“Enhancing the reproductive health of a female mammal” includes enhancing or restoring fertility, as well as alleviating menopausal disorders, including, but not limited to, somatic disorders such as osteoporosis, cardiovascular disease, somatic sexual dysfunction, hot flashes, vaginal drying, sleep disorders, depression, irritability, loss of libido, hormone imbalances, and the like, as well as cognitive disorders, such as loss of memory; emotional disorders, depression, and the like.

“Enhancing the reproductive health of a male mammal” includes restoring or enhancing fertility and/or spermatogenesis. For example, a germ-like cell made by a method of the invention may be grafted into the seminiferous epithelium of a male mammal's testes; the engrafted germ-like cell differentiates into a sperm cell in vivo, thereby restoring or enhancing spermatogenesis.

“Enhancing the reproductive health of a mammal” includes enhancing the reproductive health of a male or female mammal.

In one example of a method of treating infertility in accordance with the invention, GLCs capable of undergoing genetic recombination, meiosis and ultimately generating functional haploid cells based on selection as described above are cultured with an oocyte differentiation agent to produce an oocyte; the oocyte is fertilized in vitro to form a zygote; and the zygote is implanted into the uterus of a female subject.

Alternatively, GLCs capable of undergoing genetic recombination, meiosis and ultimately generating functional haploid cells based on selection as described above are used to induce folliculogenesis. The GLCs are engrafted into ovary tissue and differentiate into an oocyte within a follicle, thereby inducing folliculogenesis.

In representative methods of the invention useful in evaluating the effect of a composition on the reproductive health of a mammal, the composition is exposed to a germ-like cell made by a method of the invention and the effect of the composition on the germ-like cell in comparison to a control is observed.

GLCs of the invention, gametes derived therefrom, and pharmaceutical compositions comprising GLCs of the invention, may be supplied along with additional reagents in a kit. The kits can include instructions for the treatment regime or assay, reagents, equipment (test tubes, reaction vessels, needles, syringes, etc.) and standards for calibrating or conducting the treatment or assay. The instructions provided in a kit according to the invention may be directed to suitable operational parameters in the form

of a label or a separate insert. Optionally, the kit may further comprise a standard or control information so that the test sample can be compared with the control information standard to determine if whether a consistent result is achieved.

The invention is described further in the following examples, which are illustrative only and are in no way limiting.

### EXPERIMENTAL SECTION

### Part 1

### Materials and Methods for Examples 1-4

hESC Culture Conditions

BGO1 (XY) hESCs with normal karyotype were cultured on ICR mouse (Harlan, Indianapolis, http://www.harlan.com) MEF feeders inactivated by mitomycin C (Sigma-Aldrich, St. Louis, http://www.sigmaaldrich.com). The cells were cultured in 20% knockout serum replacement (KSR) stem cell medium, which consisted of Dulbecco's modified Eagle's medium/Ham's F-12 medium supplemented with 20% KSR, 2 mM L-glutamine, 0.1 mM nonessential amino acids, 50 units/ml penicillin/50 μg/ml streptomycin (Gibco, Grand Island, N.Y., http://www.invitrogen.com), 0.1 mM_-mercaptoethanol (Sigma-Aldrich), and 4 ng/ml bFGF (Sigma-Aldrich; R&D Systems Inc., Minneapolis, http://www.rndsystems.com). They were maintained in 5% CO₂ and at 37° C. Cells were passaged every 3 days by mechanical dissociation and replated on fresh feeders to prevent undirected differentiation, with daily medium changes, as previously described by our laboratory [26].

### Enrichment and Differentiation Culture Conditions

Germ-like cells were enriched in an adherent culture system by growing them on feeders or polyornithine (20 μg/ml) and laminin (5 μg/ml)-coated (feeder-free) plates with or without 4 ng/ml bFGF for 3, 10, and 30 days in 20% KSR conditioned media without passaging. Media prepared for feeder-free cultures were conditioned by exposing them to MEFs for 24 hours. Cultures were maintained in 5% CO₂ at 37° C., and medium was replaced every other day. Cells grown for 3 days with bFGF were under identical hESC maintenance conditions and are considered to be hESC controls.

### Immunocytochemistry

Cells were passaged onto glass four-chamber slides (BD Bioscience, San Jose, Calif., http://www.bdbiosciences.com) and fixed with 4% paraformaldehyde for 15 minutes. Antibodies were directed against POU5F1 (1:500; Santa Cruz Biotechnology), DDX4 (1:200; R&D Systems), MLH1 (1:200; Santa Cruz Biotechnology), and SYCP3 (1:200; Santa Cruz Biotechnology). Primary antibodies were detected using secondary antibodies conjugated to Alexa Fluor 488 or 594 (1:1,000; Molecular Probes, Eugene, Oreg., http://probes.invitrogen.com). Cell observations were made using the Olympus Ix81 (Olympus, Tokyo, http://www.olympus-global.com) with Disc-Spinning Unit and Slide Book Software (Intelligent Imaging Innovations, Santa Monica, Calif., http://www.intelligent-imaging. corn). The data were quantified using Image-Pro Plus (Media Cybernetics, Crofton, Md., http://www.mediacy.com).

### Flow Cytometry

Cells were fixed in 57%/43% ethanol/phosphate-buffered saline (PBS) for 10 minutes at room temperature. Cells were washed three times in PBS and were blocked in 6% donkey serum for 45 minutes. In feeder-free conditions, antibodies were directed against POU5F1 (1:500; Santa Cruz Biotechnology Inc., Santa Cruz, Calif., http://www.scbt.com) and DDX4 (1:200; R&D Systems). In the presence of feeders, an antibody against human nuclei (1 μl per million cells; Chemicon, Temecula, Calif., http://www.chemicon.com) was also used to prevent false positives caused by feeders. MEFs were also used as negative controls for POU5F1 and DDX4 expression. Primary antibodies were detected using fluorescently conjugated secondary antibodies Alexa Fluor 405, 488, and 647 (1:1,000; Molecular Probes). Cells were sorted and analyzed using a Dako-Cytomation CyAn flow cytometer (DakoCytomation, Glostrup, Denmark, http://www.dakocytomation.com) and FlowJo cytometry analysis software (Tree Star, Ashland, Oreg., http://www.treestar.com).

### Reverse Transcription-Polymerase Chain Reaction

RNA was extracted using the Qiashredder and RNeasy kits (Qiagen, Hilden, Germany, http://www1.qiagen.com) according to the manufacturer's instructions. The RNA quality and quantity were verified using an RNA 600 Nano Assay (Agilent Technologies, Palo Alto, Calif., http://www.agilent.com) and the Agilent 2100 Bioanalyzer. For real-time quantitative reverse transcription (RT)-polymerase chain reaction (PCR), total RNA (5 μg) was reverse-transcribed using the cDNA Archive Kit (Applied Biosystems, Foster City, Calif., http://www.appliedbiosystems.com) according to the manufacturer's protocols. Reactions were incubated initially at 25° C. for 10 minutes and subsequently at 37° C. for 120 minutes. Real-time (RT)-PCR (TaqMan; Applied Biosystems) assays were chosen for the transcripts to be evaluated from Assays-On-Demand (Applied Biosystems), a prevalidated library of human-specific quantitative PCR assays, and incorporated into 384-well Micro-Fluidics Cards. Two microliters of the cDNA samples (diluted to 50 μl) along with 50 μl of 2×PCR master mix were loaded into the respective channels on the microfludic cards, followed by centrifugation. The cards were then sealed, and real-time PCR and relative quantification were carried out on the ABI Prism 7900 Sequence Detection System (Applied Biosystems). All failed (undetermined) reactions were excluded, and ΔCt values were calculated. For calculation of relative fold change values, initial normalization was achieved against endogenous 18S ribosomal RNA using the ΔCt method of quantification (Applied Biosystems) [27]. Average fold changes from four independent runs were calculated as 2^{-ΔΔCt}. Significance was determined by running two-way analysis of variance and Tukey's pairwise (SAS Institute, Cary, N.C., http://www.sas.com) comparisons for each gene, focusing on temporal, bFGF, and feeder effects and their interactions. Qualitative RT-PCR was performed on RNA using the Qiagen OneStep RT-PCR Kit following the manufacturer's instructions. Reactions were incubated initially at 50° C. for 30 minutes and then at 95° C. for 15 minutes for reverse transcription. The PCR conditions were initiated with denaturing at 95° C. for 4 minutes followed by 34 cycles at 94° C. for 1 minute, 62° C. for 1 minute, and 72° C. for 1 minute, with a final extension at 72° C. for 10 minutes. Products were then run on 4% agarose gel and examined. The following specific primers were used for the amplification of Cyp26b1: sense, 5′TCTTTGAGGGCTTGGATCTG; antisense, 5′GAATTGGACACCGTGTTGG.

### Example 1

### Germ Cell Protein Expression in an Adherent Germ

### Cell Culture System

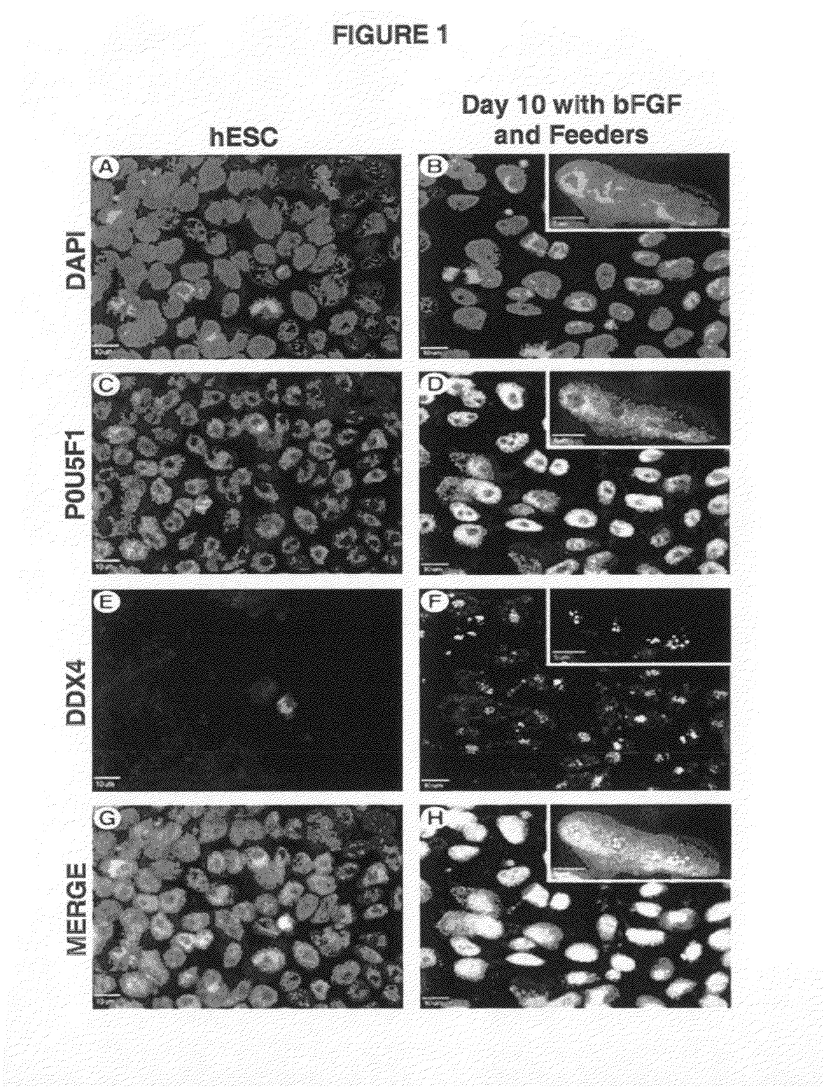

DDX4+ POU5F1+ cells were enriched on MEF feeder cocultures and on polyornithine- and laminin-coated plates for 3, 10, and 30 days without passaging under enrichment conditions. Cells grown under enrichment conditions for 3 days with bFGF were under identical hESC maintenance conditions and are considered to be hESC controls. In most cases, 4,6-diamidino-2-phenylindole (DAPI)(FIG. 1A) nuclear staining of hESCs showed colocalization with the pluripotency marker POU5F1 (FIG. 1C) and absence of the germ cell marker DDX4 (FIGS. 1E, 1G, merge). However, after 10 days of differentiation, the pluripotency marker POU5F1 (FIG. 1D) and germ cell marker DDX4 (FIG. 1F) showed nuclear colocalization with DAPI (FIGS. 1B, 1H, merge), with similar results seen at day 30. All treatments showed a subpopulation of DDX4+ POU5F1+ cells, which is indicative of early germ cell development (FIG. 1).

Unexpectedly, we observed DDX4+ POU5F1+ cells in hESC cultures; however, other groups have also noted germ-like cells in undifferentiated mouse [28] and human [3] ESCs. DDX4+ POU5F1+ cells were found to be in large clusters, which showed the potential for germ cell signaling events. DDX4 was localized to the nucleolus of DDX4+ POU5F1+ cells (FIGS. 1F, 1H, inset). In contrast, no DDX4- or POU5F1-positive staining was observed in cocultured feeder cells or other hESC-derived cells, such as human neural progenitor cells (data not shown).

### Example 2

### Temporal Effect on DDX4+ POU5F1+ Expression

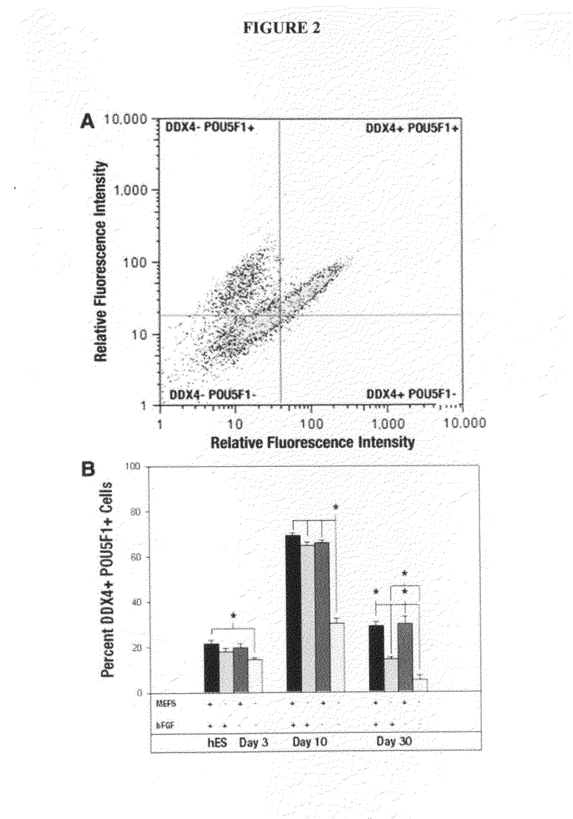

Immunocytochemistry showed enhanced germ-like marker expression under enrichment conditions; therefore, we used flow cytometry to quantify the enrichment of DDX4+ POU5F1+ cells. Flow analysis further confirmed that a population of cells were indeed positive for both DDX4 and POU5F1 (FIG. 2A). Flow analysis showed that there was a significant (p less than 0.05) temporal effect, with an increase in DDX4+ POU5F1+ cell percentage at day 10 (average of treatments, 57.62±9.20) compared with the ESC control, day 3 (average of treatments, 18.47±1.56), and 30 (average of treatments, 19.62±5.98), with and without feeders and bFGF (FIG. 2B). bFGF played a significant role in increasing the percentage of DDX4+ POU5F1+ cells, but only under feeder-free conditions (FIG. 2B). There was a significant (p less than 0.05) increase in the percentage of DDX4+ POU5F1+ cells at days 10 (34.63%) and 30 (9.16%) in the presence of bFGF. However, bFGF had no significant effect when hESCs were cultured on feeders.

Within day 10 samples, cells without bFGF and feeders showed significantly (p less than 0.05) reduced numbers of DDX4+ POU5F1+ cells (FIG. 2B). Samples within day 30 were also compared, and treatments without MEFs showed reduced numbers of DDX4+ POU5F1+ cells irrespective of the presence of bFGF. This indicates that feeder cell contact plays a role in germ cell enrichment, since cells grown without feeders were grown in MEF-conditioned media and would carry feeder-derived soluble signaling factors.

### Example 3

### MEF Feeders Increased Germ-Like Gene Expression

### Premigratory and Migratory Stage Gene Expression.

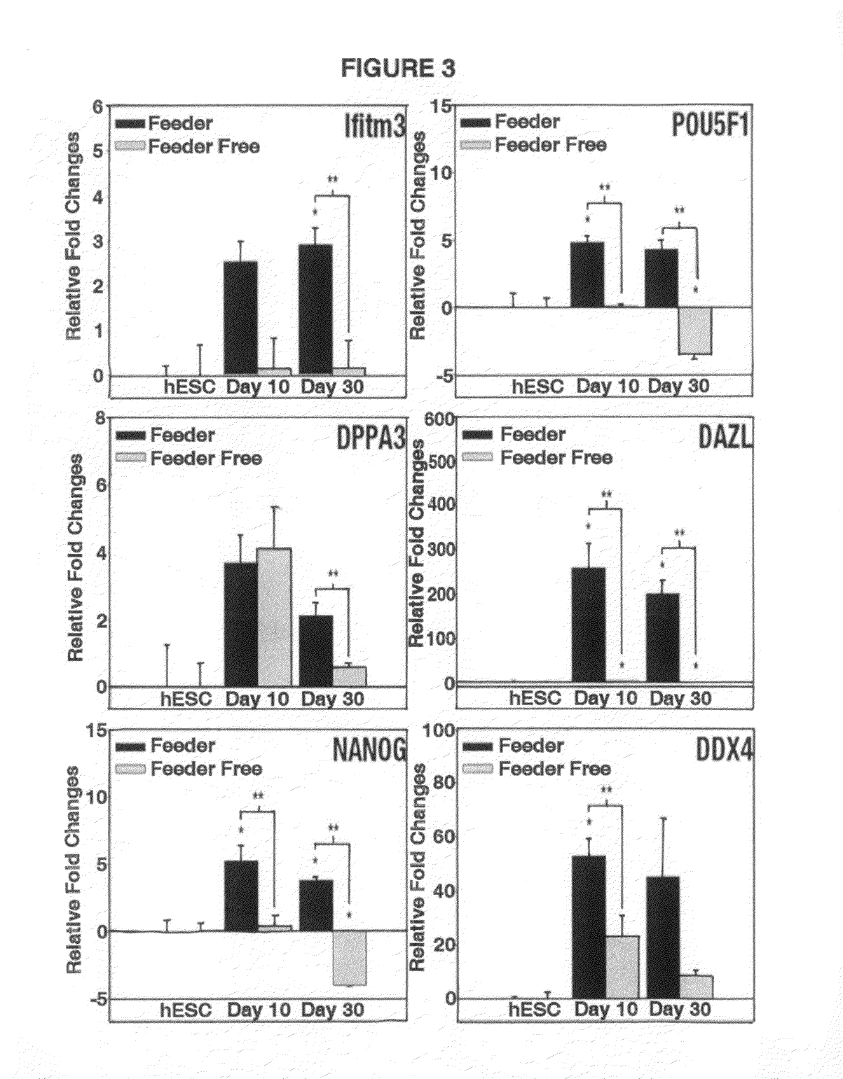

Because of bFGF optimizing the performance of feeder-free cultures by enriching DDX4+ POU5F1+ cells, germ cell gene expression was examined only in the presence of bFGF. Significant increases in premigratory (Ifitm3, DPPA3, POU5F1, and DAZL) and migratory (NANOG and DDX4) germ cell gene expression were observed for cultures both with and without feeders at days 10 and 30; however, cultures grown on feeders consistently showed significantly higher germ cell gene expression than feeder-free conditions, regardless of day (FIG. 3). Cells cultured on feeders had significantly higher POU5F1, DAZL, and NANOG gene expression (p less than 0.05) at both day 10 and day 30, relative to feederless counterparts (fold changes are shown in FIG. 3). Feeder cell-cultured conditions also produced significant (p less than 0.05) increases for DDX4 expression at day 10 and Ifitm3 and DPPA3 expression at day 30 in comparison with groups without feeders (FIG. 3). Overall, treatments with feeders showed noted increases in premigratory and migratory gene expression relative to the treatments without feeders. In fact, gene expression for DAZL was several hundredfold higher when cells were on feeders. In addition, treatments without feeders showed decreased gene expression relative to hESCs for POU5F1, DAZL, and NANOG at day 30, whereas a decrease in germ cell gene expression was never observed in feeder culture conditions. When enriched feeder cell cultures were compared with undifferentiated hESCs, DAZL and NANOG, genes expressed during premigratory and migratory stages, were upregulated in day 10 and day 30 cultures (FIG. 3). Other premigratory genes, POU5F1 and DDX4, were also higher at day 10, and Ifitm3 was higher at day 30 relative to hESC control. In contrast, cultures without feeders showed only limited increases. DAZL in feederfree cultures was upregulated only at day 10 (FIG. 3). All other genes (Ifitm3, POU5F1, DPPA3, NANOG, and DDX4) showed no significant increase relative to hESC controls. This proved that feeder culture conditions caused increased premigratory and migratory germ cell gene expression over time, whereas cultures without feeders showed limited temporal increases.

### Genital Ridge, Spermatogonia, and Meiotic Stage Gene Expression.

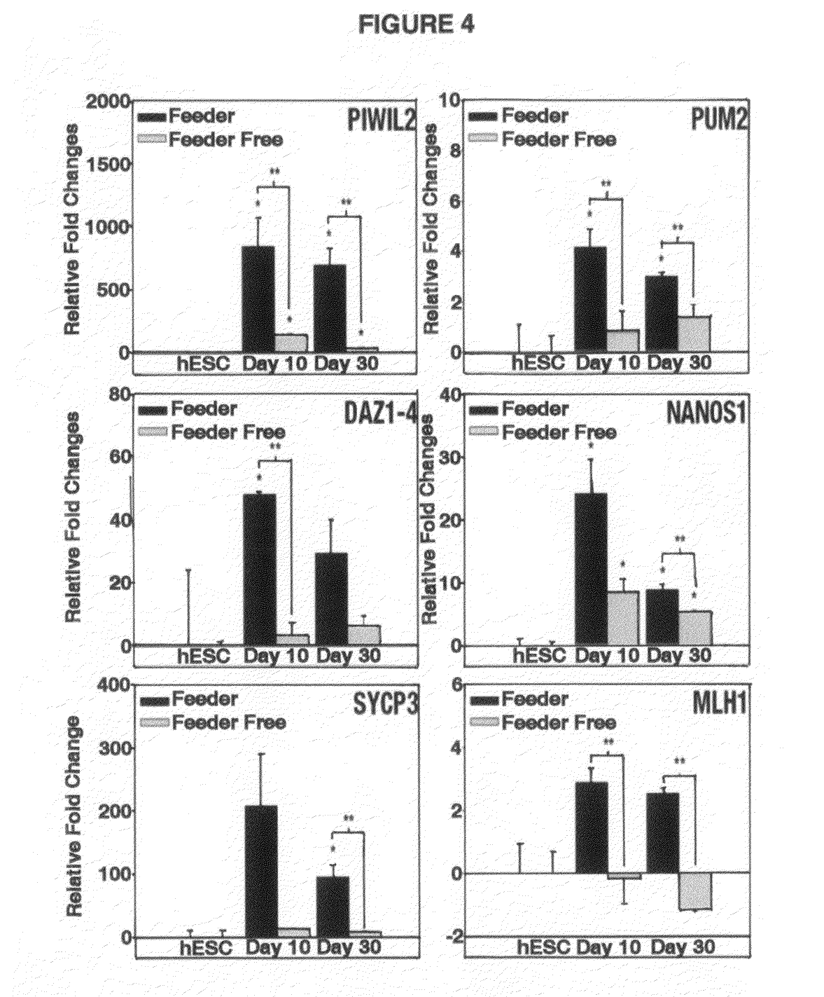

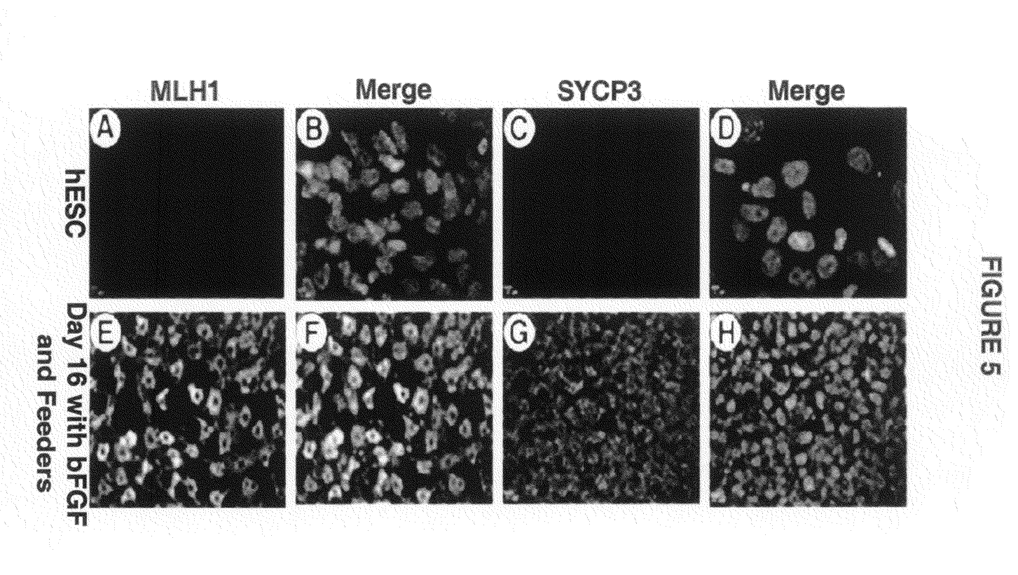

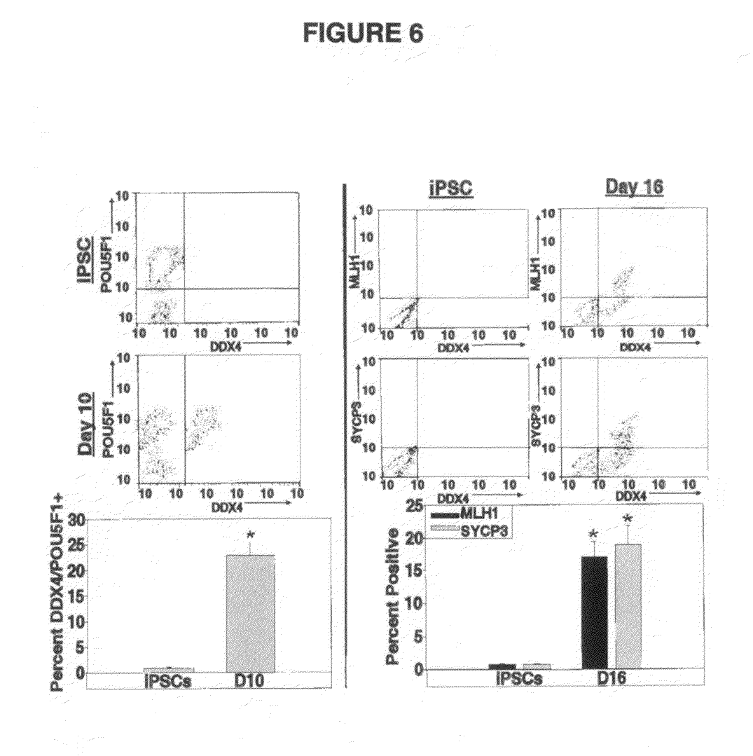

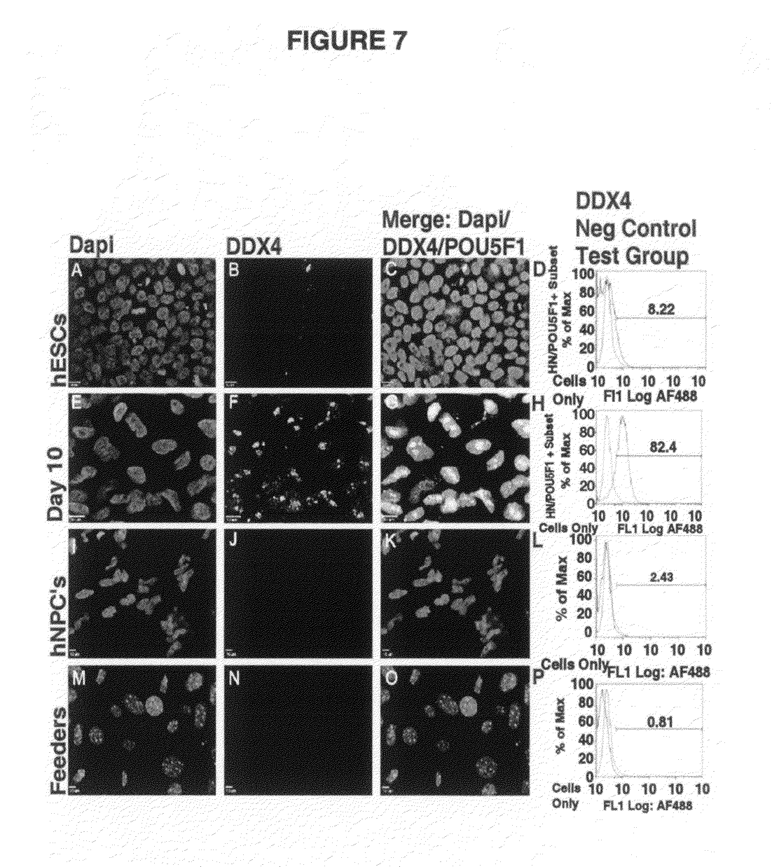

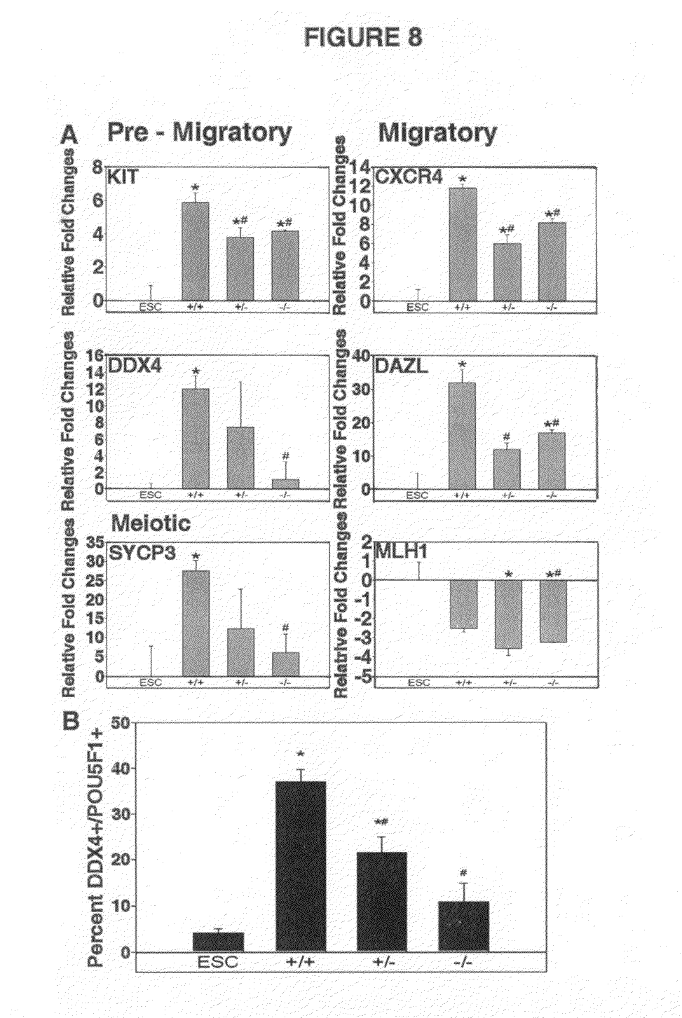

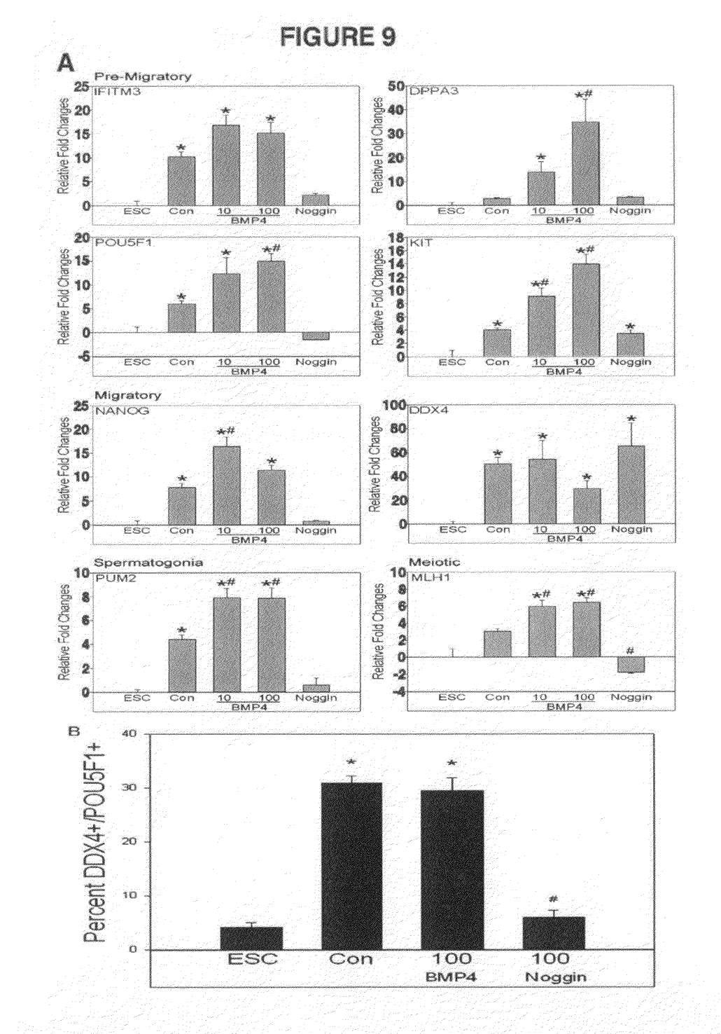

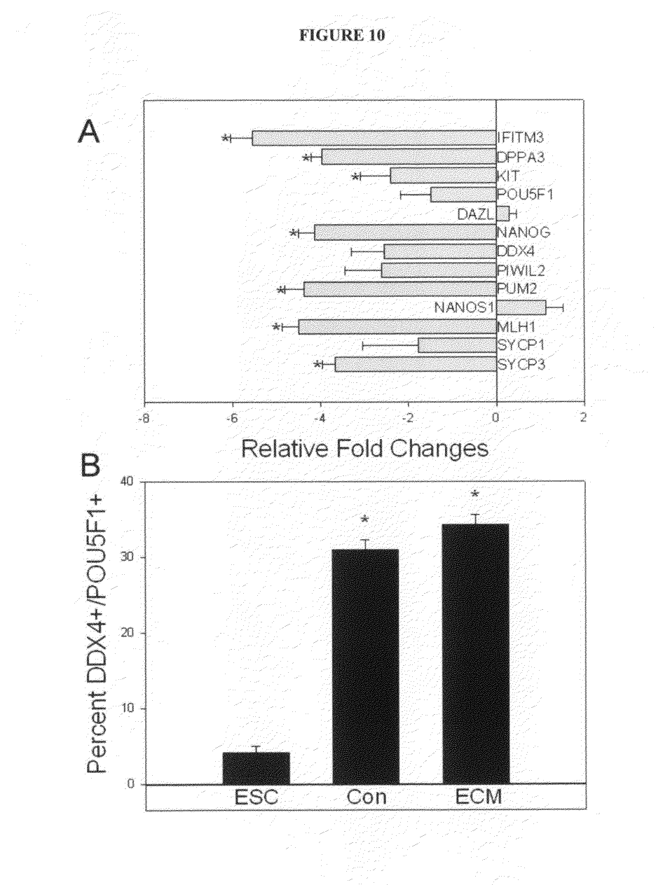

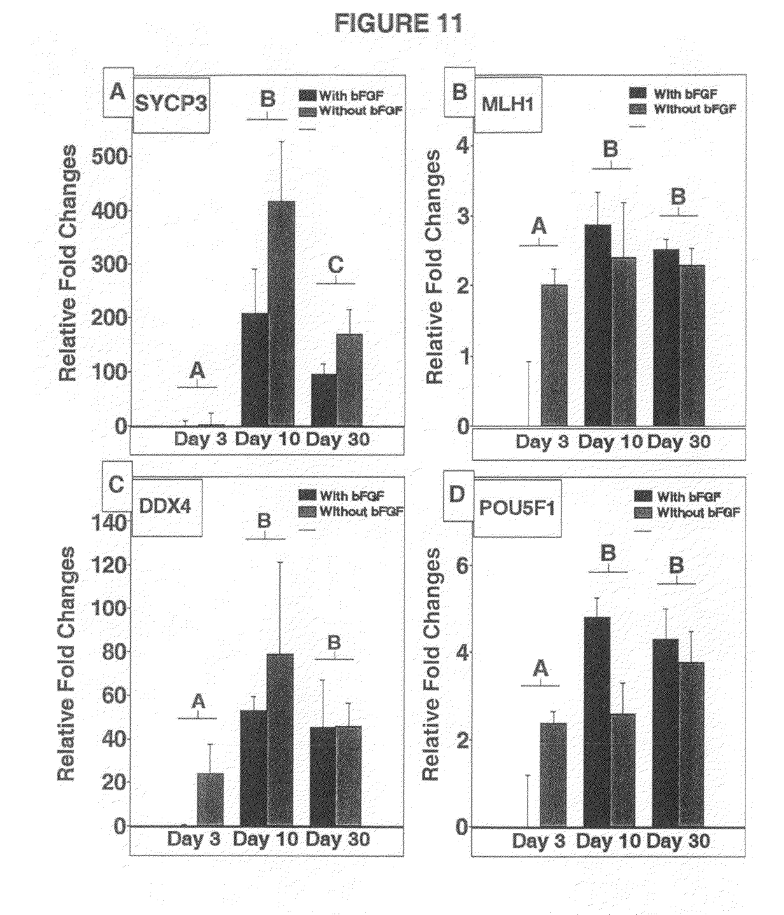

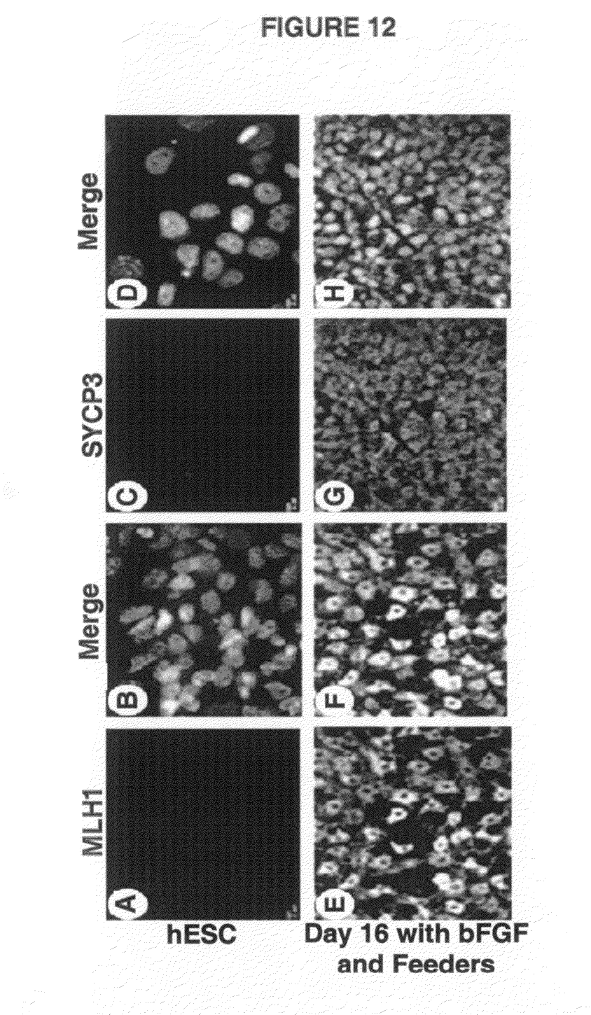

We then asked whether our culture system upregulated the expression of postmigratory genes of the genital ridge (PIWIL2), spermatogonia (PUM2, DAZ1-4, and NANOS1), and meiotic (MLH1 and SYCP3) phases of germ cell development. Therefore, we compared contemporary cultures by quantitative RT-PCR and found that cultures exposed to feeders consistently showed significantly higher germ cell gene expression than feeder-free cultures. Feeder cell cultures significantly (p less than 0.05) increased PIWIL2, PUM2, and MLH1 gene expression in day 10 and 30 cultures, relative to their contemporary without feeder cultures (FIG. 4). The feeder treatment groups also produced an increase in the DAZ1-4 cluster (DAZ1, DAZ2, DAZ3, DAZ4) at day 10 and NANOS1 and SYCP3 at day 30 (FIG. 4). Some treatments with feeders produced highly significant changes in postmigratory gene expression; PIWIL2, SYCP3, and the DAZ family cluster had at least a 10-fold increase in expression (p less than 0.05) relative to feeder-free culture. The upregulation of these postmigratory genes is indicative of advanced stages of differentiation when MEF feeder cells are present. Postmigratory gene expression was increased over time in MEF feeder culture conditions compared with undifferentiated hESC cultures, but less so in feeder-free conditions. In feeder culture conditions, expression of PIWIL2, PUM2, and NANOS1 was significantly (p less than 0.05) higher at days 10 and 30 than in hESCs (FIG. 4). SYCP3, a known meiotic marker, was upregulated at day 30 relative to hESCs. Although feeder-free cultures generally showed less expression for these genes compared with MEF-grown cells, they were upregulated compared with undifferentiated hESCs for the genes NANOS1 and PIWIL2 at days 10 and 30 (FIG. 4). In contrast to the MEF-included culture, PUM2 and SYCP3 showed no upregulation at day 10 or day 30 in feeder-free conditions. This suggests that feeder conditions generally cause a temporal increase in postmigratory gene expression, whereas feeder-free conditions fail to do so.

### Example 4

### Expression of Meiotic Markers in Culture

Increased gene expression of the meiotic genes MLH1 and SYCP3 indicated potential entry into meiosis; however, normally this does not occur in male germ cell development until puberty. Cyp26b1, a retinoic acid degrading enzyme, regulates sex-specific timing of meiotic entry and inhibits meiosis in male mice, whereas male Cyp26b1-knockout mouse germ cells enter meiosis in prenatal stages similar to female counterparts [29, 30]. Our RT-PCR results suggested that Cyp26b1 is not expressed in cultures of all treatments (gel not shown). The absence of Cyp26b1 may contribute to the onset of meiotic gene expression in cultures of germ-like cells. To determine whether germ-like cells undergo meiosis in culture, hESCs were differentiated for 10, 16, and 30 days on MEFs in the presence of bFGF and immunostained for MLH1 and SYCP3.

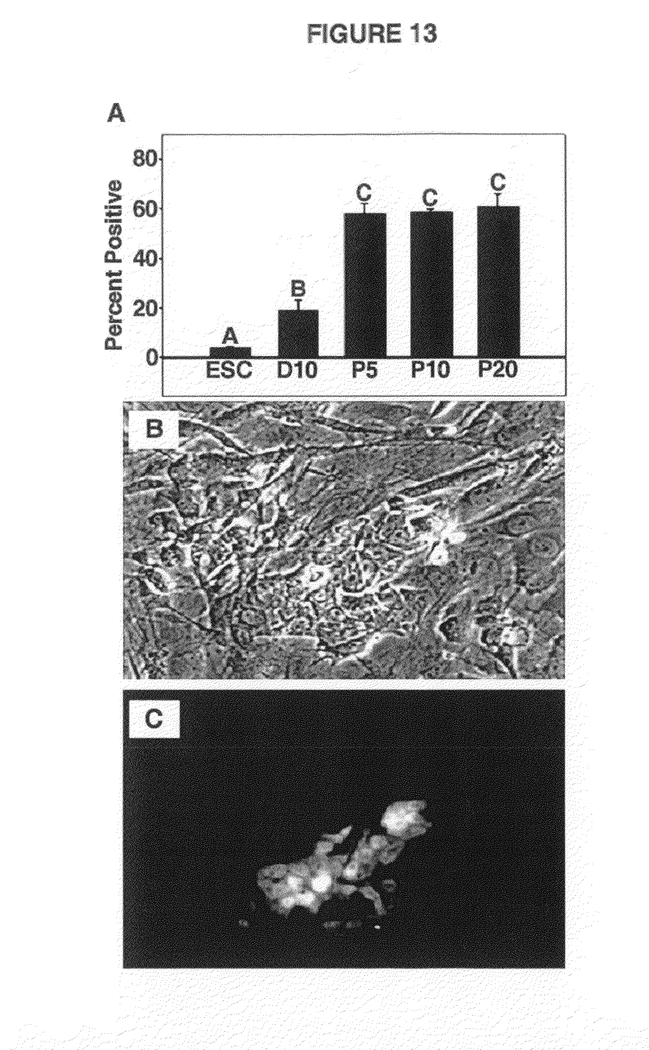

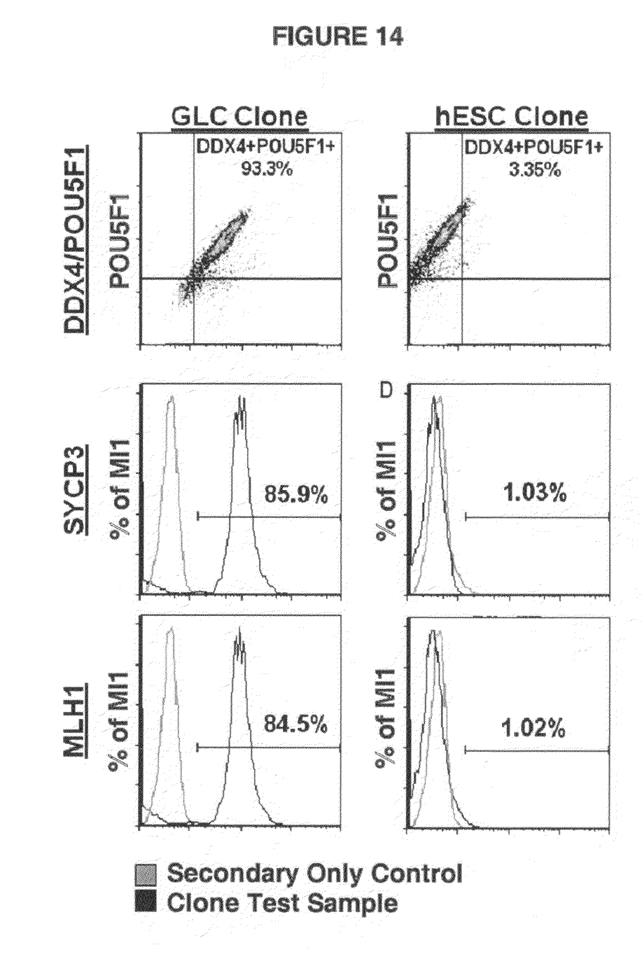

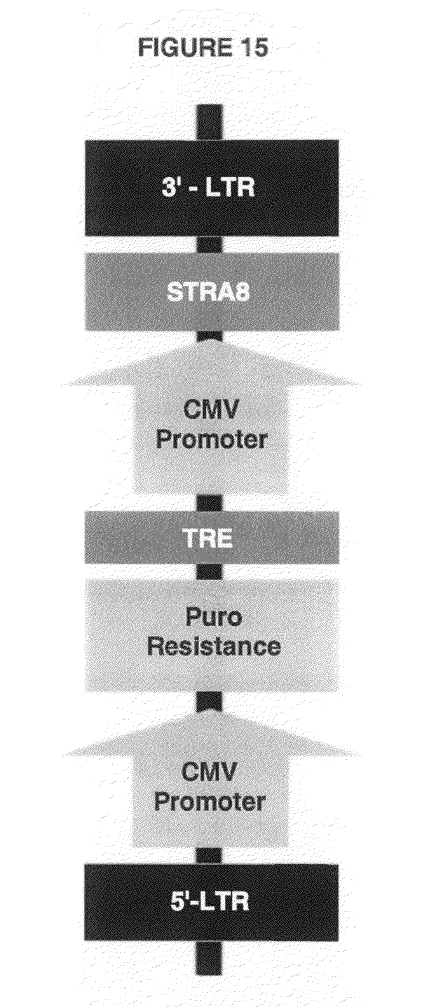

Immunostaining showed that 90% of day 16 cells were positive for MLH1 (FIGS. 5E, 5F) and SYCP3 (FIGS. 5G, 5H) proteins, whereas no expression of either marker was found in hESCs (FIG. 5A-5D), day 10 (data not shown) cells, or day 30 cells. In addition, staining was localized to the nucleus, which correlates with their known role in chromosome segregation during meiosis [17, 20, 31], suggesting that germ-like cells have the potential to undergo meiosis in culture.

### Example 5

### GLC and Meiotic Marker expression Form Induced Pluripotent Stem Cells (iPSC)

Recently several groups have demonstrated that expression of various combinations of six transcription factors (Oct4, Sox2, Nanog, Klf4, cMyc and Lin28) can induce human somatic cells into a pluripotent state where they exhibit the essential characteristics of hESC. These iPSC demonstrate a normal karyotype, express telomerase activity, and express markers that characterize hESC. Importantly these cells also maintain the potential to differentiate into cell types of all three germ layers. Using a lentiviral system we have transduced IMR90 fibroblast cells using an EF1α promoter to drive expression of the six transcription factors and plated the cells onto mouse fibroblasts in hESC medium as previously described. Preliminary results have repeatedly demonstrated the formation of colonies of cells morphologically similar to hESC colonies. These iPSC were cultured in the same manner as pluripotent embryonic stem cells (example 3 and 4) but were from lower passage number than hESC (less than 10 passage) to differentiate these iPSC towards DDX4+ POU5F1+ cells and were enriched on MEF feeder cocultures and on polyornithine- and laminin-coated plates for 10 days without passaging under enrichment conditions (FIG. 6). In addition further differentiation (day 16) produced MLH and SYCP3) positive cells (FIG. 6). Therefore the methods describe here work equally as well in differentiating any type of pluripotent stem cells (ESC or iPSC)

### Example 6

### Pluripotent Stem Cells Derived Haploid Germ Cells

hESCs were differentiated for 5 to 15 days in 20% KSR media in the presents or absents of feeders or feeder derivatives as described in West et al. 2008 [1]. Cells were then exposed to fetal bovine serum differentiation media (1% to 50% fetal bovine serum, 95% to 50% Dulbecco's Modified Eagle Medium: Nutrient Mixture F-12 (DMEM/F12), 0% to 10% Penicillin/Strptomycin, 0% to 10% 2 mM L-Glutamine, 0% to 10% 10 mM MEM Non-Essential Amino Acids, 0 to 500 ug/ml of basic fibroblast growth factor and 0 to 1 ul/ml 1 M β-Mercaptoethanol) for 5 to 50 days without passaging with media changes daily to every 7 days at 35 to 40° Celsius and 2 to 10% CO₂. Utilizing this system, 0 to 12% of germ-like cells demonstrated haploid character. In addition, these cells expressed advanced differentiation markers including acrosin, haprin, protamine 1, protamine 2 and RNF17.

Discussion of the Experimental Results of Examples 1-6 We used hESCs as a model to understand the differentiation process toward the germ cell lineage. After examining multiple treatment variations, we were able to produce a 69% DDX4+ and POU5F1+ germ-like cell population when culturing hESCs on MEF feeder cells with bFGF-supplemented media. To our knowledge this is the most uniform population of germ-like cells generated from hESCs reported to date. In previous studies, hESCs formed more heterogeneous populations, and the best results previously published were no higher than 35% of the population being DDX4-positive [3, 14] in embryoid body differentiation systems, in contrast to the adherent culture system described here. Chen et al. used a human adherent germ cell differentiation culture system; however, they did not determine in a quantifiable method the number of germ-like cells produced, nor did they examine the role of feeder cells or bFGF-supplemented media [32].

Feeder cells and bFGF have important roles in maintaining both ESCs and primordial germ cells [33], but their role in germ cell differentiation has not been investigated. Here, the role of bFGF proved to be of even greater importance under feeder-free conditions, with an increase of more than 100% in DDX4+ POU5F1+ cells in feeder-free conditions with bFGF at days 10 and 30, relative to feeder-free conditions without bFGF (FIG. 2). Because the feeder-free culture uses MEF-conditioned media, this suggests that the levels of MEF-derived bFGF are not optimal for germ cell differentiation. At the same time, it is unlikely that bFGF is the only factor associated with the MEFs affecting our results. In addition to producing bFGF [34], MEF feeders produce Kit ligand (also known as stem cell factor), which is linked to germ cell proliferation and development [35]. These and other factors are logical targets for further understanding of germ cell differentiation in this system. Successful maintenance of pluripotent hESCs is believed to be due to the inhibitory effect of bFGF on the bone morphogenetic protein (BMP) family pathway [36]. This is the same signaling family proven to be essential for germ cell development in vivo [8, 36, 37]; it is present in our system as a part of the knockout serum replacement [36] and is thought to be the potential cause of the “undirected” differentiation and enrichment. However, linagespecific induction by BMP family members is concentration dependent [38, 39] and unknown in our extended cultures. Therefore, the in vitro conditions in our culture, with the presence of MEFs, may create a microenvironment that limits the differentiation into other cell types [40] and/or promotes germ cell differentiation.

DDX4 has proven to be a robust germ cell marker, being one of the few genes determined to be restricted to the germ cell linage in humans [10]. However, the function and localization of the DDX4 protein are not fully understood, nor has it been well characterized. We showed novel localization of the DDX4 protein to the nucleolus, which could indicate a functional role in human germ cell development. The mouse DDX4 homolog, mouse vasa homolog, has traditionally been noted as being cytoplasmically localized; although its function is unclear, it is critical in maintaining fertility [12, 41]. DDX4 is an RNA helicase and might be associated with germ cell-specific RNA molecules that are localized to the nucleolus. Several other DEAD-box protein family members (e.g., DDX47) have also been found localized to the nucleolus, but they have not been shown to be associated with germ cell development [42]. A better understanding of this germ cell-specific protein and its localization is clearly needed, and our culture system can provide an important in vitro model for this investigation. Previous studies have shown that subpopulations of mouse [28] and human [3] ESCs express germ cell markers, which was further confirmed in this study with the expression of DDX4 in undifferentiated hESCs. The overlapping expression of germ cell markers between germ cells and stem cells may indicate that DDX4 has a function both in early germ cells and in maintaining pluripotent cell types.

In Examples 1-4, changes in the expression of six germ cell premigratory and migratory genes and six postmigratory germ cell genes were monitored during the temporal differentiation of hESCs in this adherent system. The presence of feeder cells caused dramatic increases in premigratory, migratory, and postmigratory germ cell gene expression compared with the feederless conditioned medium treatment. Once again, the mechanism by which feeder cells cause this increase is not clear, yet important. Because cells in the feeder-free conditioned medium group were less germ-like, we hypothesize that the effect of increased germ cell gene expression is mediated through hESCMEF cell contact.

Overall, 10 days of differentiation proved to be optimum, with noted increases in both germ cell gene and protein markers at this time. The fact that early to late germ cell markers were expressed at this time suggests that day 10 cells were not a homogeneous population. Also, unexpectedly, the early germ cell-specifying gene Ifitm3 increased only modestly over time, and DPPA3, another germ cell-specifying gene, showed no increased expression over time. The lack of increased expression of some early germ cell-specifying genes may be due to the fact that they may already be expressed at some level in hESCs [3]. This has led some to suggest that initial germ cell programming may already be activated in undifferentiated hESCs [3]. If this were the case, then increased expression, as we observed here, of early germ cell genes such as DPPA3 would not be expected. POU5F1 and NANOG were more highly expressed at day 10 than the starting hESC populations in treatments with feeders, which was unanticipated since these genes are highly expressed in hESCs [43]. However, POU5F1 expression levels have been linked with specific differentiation pathways. Loss of POU5F1 has been shown to result in spontaneous differentiation, but upregulation results in differentiation into primitive endoderm [44]. Therefore, POU5F1 expression may differ depending on the differentiating cell type, and thus differentiating germ cells may require relatively high expression.

Significant gene and protein expression of the meiotic markers SYCP3 and MLH1 was observed under differentiation conditions with feeders. SYCP3 and MLH1 are both early meiotic markers being expressed in prophase, suggesting entry into meiosis [16-20, 31]. The increased expression of meiotic genes after only 10 days of differentiation and the presence of protein after 16 days are unexpected given that meiosis- and meiosisassociated proteins are normally not observed until after puberty.

Intuitively, these proteins would not be expected to be present after only 16 days of hESC differentiation in vitro. However, this rapid entry into meiosis is consistent with human [3, 14] and mouse [21] ESC-derived germ-like cells. Geijsen et al. [21] noted expression of the haploid germ cell marker FE-J1 in mESC-derived germ-like cells after 13 days of differentiation, whereas Clark et al. [3] observed some meiotic activity in hESC-derived cells. The SYCP1 gene and cytoplasmic SYCP3 protein were observed, but the MLH1 protein was not present after 14 days of hESCs differentiation [3]. However, under our conditions SYCP3 and MLH1 were localized in the nucleus of more than 90% of the germ-like cells, and both meiotic markers were absent in undifferentiated hESCs. It is encouraging that these proteins were found in the nucleus, and potentially, in the future, human haploid cells may be generated from these cultures. We also found an absence of expression of Cyp26b1, a meiotic initiation inhibitor, further suggesting that regulation of male germ cell meiotic events normally observed in vivo may not be mimicked in our cell cultures. In support of our findings, germ cells from Cyp26b1-knockout males [29, 30] and male germ cells that were ectopically developed, and presumably not exposed to Cyp26b1 [45, 46], have been found to directly enter meiosis, similar to those of wild-type females. Our study demonstrates that the timing of meiotic activity in germ-like cells is early relative to in vivo counterparts, and further inspection for normal meiotic activity is clearly needed. Nonetheless, abbreviated germ cell differentiation may ultimately prove to be an advantage from a therapeutic perspective if germ cell differentiation and maturation can be accelerated. Therefore, in the experiments of Examples 1-6, using a novel adherent culture system with MEF feeder cells and bFGF, we have demonstrated successful and reliable production of a population where 69% of cells express germ-like character (DDX4+ POU5F1+) by immunocytochemistry, flow cytometry, and quantitative RT-PCR. Enriched cultures showed progressive differentiation with the expression of premigratory, migratory, and postmigratory genes, with some genes being expressed several hundredfold higher than their hESC counterparts. These enriched cultures ultimately demonstrated advanced levels of differentiation, with 90% of cells expressing the meiotic markers SYCP3 and MLH1 and can form haploid cells. In additional experiment X demonstrates that GLC and differentiated cells expressing SYCP3 and MLH1 can also be generated from induced pluripotent stem cells. This robust system clearly has the potential for use in parsing the factors involved in the differentiation of hESCs down the germ cell lineage.

### Part 2

### Overview

Investigation of early human germ cell developmental niche has been hampered, due in part to a lack of biological resources, and therefore the majority of mammalian germ cell developmental studies have been conducted in the mouse. Mouse germ-like cells derived from mouse embryonic stem cells (mESC) undergo meiosis, elongate and even produce live offspring (Geijsen, et al., 2004, Hubner, et al., 2003, Nayernia, et al., 2006, Toyooka, et al., 2003). However, murine studies may not always directly translate to advances in human germ cell development, given that similar results using human embryonic stem cells (hESCs) have not been reported. This may reflect species specific differences in germ cell development and is indicative of the challenge in directly translating mESC germ cell results to hESC germ cell development. These differences highlight the need to directly investigate germ cell signaling factors and their relation to early human germ cell developmental events in human cells.

In vivo, several growth factors have proven to be essential in the proper differentiation of developing germ cells in the mouse. These are logical initial signaling factors to investigate in human germ cell development. Two of these factors are KIT ligand (KITL) (Matsui, et al., 1990) and bone morphogenetic protein 4 (BMP4) (Lawson, et al., 1999), the transforming growth factor beta (TGF-β) superfamily member. Interruption of normal KITL expression, which is expressed by the somatic tissue along the early germ cell migratory path in both mouse (Matsui, Zsebo and Hogan, 1990) and humans (Hoyer, et al., 2005), or its receptor KIT results in a loss of normal migration and proliferation in early mouse germ cells (Matsui, Zsebo and Hogan, 1990, Runyan, et al., 2006, Chabot, et al., 1988, Geissler, et al., 1988, Mahakali Zama, et al., 2005). KITL/KIT signaling is also important for postnatal germ cell development, specifically in the differentiation of spermatogonial stem cells (SSCs) into spermatids (Yoshinaga, et al., 1991, Packer, et al., 1995, Sette, et al., 2000). Inhibition of KITL/KIT signaling activity results in the loss of differentiated type A spermatogonia and all downstream derivatives, leading to sterility (Yoshinaga, et al., 1991, Manova, et al., 1993). In addition, KITL/KIT signaling maintains extended mouse primary germ cell cultures (Godin, et al., 1991). KITL and KIT have both been shown to be present in the human adult testes with abnormal expression being associated with sub-fertility (Feng, et al., 1999, Sandlow, et al., 1997, Sandlow, et al., 1996). Similar to KITL/KIT signaling, BMP4 plays a significant role in early germ cell development in the mouse. In the gastrulating mouse embryo, germ cell specification begins with BMP4 (Lawson, et al., 1999, Ying, et al., 2001, Ying and Zhao, 2001) signaling from the extra-embryonic ectoderm to the proximal region of the epiblast. BMP4 signaling molecules then bind to BMP receptors and activate genes responsible for initial germ cell development. Inhibition of BMP4 signaling has resulted in a partial or complete loss of murine germ cell formation (Lawson, et al., 1999, Chang and Matzuk, 2001, Hayashi, et al., 2002, Okamura, et al., 2005), yet the function of BMP4 in human germ cell development has not been established.

In the experiments of Examples 1-4, mouse embryonic fibroblast cell contact with hESC was essential for germ cell formation. Further, differentiation of hESCs in feeder conditioned media on poly-ornithine and laminin coated plates resulted in a significant reduction in germ cell gene expression. One potential source of germ cell signaling is the feeder extracellular matrix (ECM). The ECM plays a significant role in differentiation of early cell types (Kihara, et al., 2006, Naugle, et al., 2006, Suzuki, et al., 2003) including ESCs (Chen, et al., 2007, Kawasaki, et al., 2000, Gong, et al., 2008, Rust, et al., 2006) into specific lineages. The role of the ECM in germ cell adhesion and migration has been well studied (Pereda, et al., 2006, Bendel-Stenzel, et al., 1998). However, the ECMs direct involvement in gem cell differentiation remains to be elucidated.

In the experiments of this section, we use the adherent hESC to germ cell differentiation culture system of Examples 1-4 to determine the effect of KITL and BMP4 on enrichment and differentiation of germ-like (DDX4+ POU5F1+) cells. Using KITL knockout feeders, we demonstrate that KITL plays a significant role in enrichment of germ-like cells in vitro with the loss of its expression causing a significant decrease in DDX4+ POU5F1+ cells and gene expression with some changes being >20 fold. Results also indicated the importance of BMP signaling in enhancing germ cell development with inhibition of endogenous signaling by the BMP receptor antagonist noggin causing a significant decrease in DDX4+ POU5F1+ cells and germ cell gene expression. These findings being supported by elevated germ cell gene expression caused by exposure to exogenous BMP4. Additionally, the differentiation of hESCs on mouse feeder ECM caused a reduction in germ cell gene expression and indicates a dynamic cell signaling process between feeder cells and hESCs. These data suggest that hESC derived germ cells provide a robust and much needed system to study human germ cell signaling and development.

### Materials and Methods for Examples 7-9

hESC Culture Conditions
BGO1 (XY) hESC with normal karyotype were cultured on ICR mouse embryonic fibroblast (MEF; Harlan, Indianapolis, Ind., USA) feeders inactivated by mitomycin C (Sigma-Aldrich, St. Louis, Mo., USA). The cells were cultured in 20% KSR stem cell media, which consists of Dulbecco's modified Eagle medium (DMEM)/F12 supplemented with 20% knockout serum replacement (KSR), 2 mM L-glutamine, 0.1 mM non-essential amino acids, 50 units/ml penicillin/50 μg/ml streptomycin (Invitrogen, Carlsbad, Calif., USA), 0.1 mM (3-mercaptoethanol (Sigma-Aldrich) and 4 ng/ml bFGF (Sigma-Aldrich and R & D Systems, Minneapolis, Minn. USA). They were maintained in 5% CO₂ and at 37° C. Cells were passaged every 3 days by mechanical dissociation, re-plated on fresh feeders to prevent undirected differentiation with daily media changes as previously described in our laboratory (Mitalipova, et al., 2003).

### Enrichment and Differentiation Culture Conditions

As previously described in Examples 1-4, germ-like cells were differentiated in an adherent culture system by growing them on ICR MEF feeders (Harlan) in 20% KSR media for 10 days without passaging. Cultures were maintained in 5% CO₂ at 37° C. and media was replaced every other day to stimulate germ cell signaling. In studies using
BMP4 and noggin, hESCs were exposed to 10 or 100 ng/ml recombinant human BMP4 (R & D Systems) for the first 3 days of differentiation or continually exposed to 100 ng/ml of recombinant human noggin (R & D Systems) for 10 days under standard differentiation conditions. Control cells were differentiated under standard conditions in the absence of both BMP4 and noggin. The ECM was prepared as previously described (Gospodarowicz, et al., 1980); briefly, mitotically inactivated ICR mouse feeders at a density of 12,000 cells/cm² and maintaining them in culture for 4 days. Lysis of confluent feeder layers exposed the ECM as a substrate for cell attachment. Feeder cells were then washed with PBS, incubated for 3 minutes in 0.02M NH₄OH and washed 3 times in PBS. hESC were seeded on the ECM and differentiated in 20% KSR media as previously described.

To determine the ability of KITL to modulate germ-like cell differentiation, hESC were differentiated on Kitl^{sl-gb} feeders from wild-type, heterozygous and homozygous mutant mice. The mice were originally obtained from the MRC Radiobiology Unit (Chilton, Didcot, UK) and have been maintained on C3H/HeNCR background for more than 20 generations (M. Bedell, personal communications). The mice used in this study were previously demonstrated to have the Kitl^{Sl-gb} deletion, which caused a complete loss in Kitl mRNA expression (Rajaraman, et al., 2002) and primordial germ cell formation by 11.5 day post coitum (dpc) (Mahakali Zama, Hudson and Bedell, 2005). Briefly, Kitl^{sl-gb} heterozygous mice were intercrossed and offspring were collected at day 13.5 dpc. The fibroblast cells were individually isolated from each fetus to prevent cross contamination between homo- and heterozygous individuals. Genotyping was done for each individual by extracting genomic DNA using a 50 mM KCl, 10 mM Tris-HCl pH 8.3, 2 mM MgCl₂, 0.1 mg/ml gelatin, 0.45% Nonidet P40, and 0.45% Tween 20 buffer. Samples were then heat inactivated at 95° C. for 10 min. PCR amplification was performed using primers that expand the gb deletion breakpoint. The primers used were: gbA 5′-TGTATCAAAAGGGTCGGGAC-3′, gbB 5′-AGTTCAGTCATAGATTGGAG-3′; gbC 5′-ATTGCTGTACTTGCTGCCTG-3′. Amplification products were analyzed on a 7% acrylamide gels.

To assess methylation status of germ-like cells during development, cells were passaged onto 4-well chamber slides (BD Falcon, Franklin Lakes, N.J., USA), differentiated for 10 days and stained for 5-methylcytidine every day starting at day 4. Comparisons were made by visual inspection.

### Immunocytochemistry

Cells were passaged onto glass 4-well chamber slides (B D Falcon) and fixed with 4% paraformaldehyde for 15 minutes. Antibodies were directed against POU5F1 (Santa Cruz Biotechnology, Santa Cruz, Calif. USA, 1:500) and DDX4 (R & D Systems, 1:200). Primary antibodies were detected using secondary antibodies conjugated to Alexa Flour 488 or 594 (Invitrogen, 1:1000). Immunoflurescence imaging was done using the Olympus Ix81 with Disc-Spinning Unit and Slide Book Software (Intelligent Imaging Innovations, Denver, Colo., USA).

Methylation was assessed by fixing cells with 70% ethanol for 30 minutes. Cells were then treated with 2N HCL and 0.5% triton-X 100 solution for 30 minutes, which was neutralized with a 0.1M Na₂BO₇ solution for 10 minutes. Antibodies were directed against 5-Methylcytidine (Santa Cruz, 1:500) and DDX4 (R & D Systems, 1:200), which were detected using secondary antibodies conjugated to Alexa Flour 488 or 594 (Invitrogen, 1:1000). Cells were observed using the Olympus 1x81 with Disc-Spinning Unit as mentioned before.

### Flow Cytometry

Cells were fixed in 57/43% ethanol/PBS for 10 minutes at room temperature. Cells were washed 3 times in PBS and were blocked in 6% donkey serum for 45 min. Antibodies were directed against POU5F1 (Santa Cruz Biotechnology, 1:250) and DDX4 (R & D Systems, 1:200). Due to the presence of feeders, an antibody against Human Nuclei (Millipore, Billerica, Mass., USA, 1 μl per million cells) was also used to prevent false positives caused by feeders. MEFs and ENStem human neural progenitors (Millipore) were used as negative controls for POU5F1 and DDX4 expression. Primary antibodies were detected using fluorescently conjugated secondary antibodies Alexa Flour 405, 488 and 647. (Invitrogen, 1:1000). Cells were analyzed using a Dakocytomation Cyan (DakoCytomation, Carpinteria, Calif., USA) and FlowJo Cytometry analysis software (Tree Star, Ashland, Oreg., USA). Significance was determined by running a 2-way ANOVA and Tukey's Pair-Wise (SAS, Cary, N.C., USA) comparisons for each treatment looking at the effects of BMP4, Noggin, KITL and MEF ECM differentiation. Treatments were a p-value was <0.05 were considered to be significantly different.

### Real-Time PCR

RNA was extracted using the Qiashredder and RNeasy kits (Qiagen, Germantown, Md., USA) according to the manufacturer's instructions. The RNA quality and quantity was verified using a RNA 600 Nano Assay (Agilent Technologies, Santa Clara, Calif., USA) and the Agilent 2100 Bioanalyzer. Total RNA (5 μg) was reverse-transcribed using the cDNA Archive Kit (Applied Biosystems Inc., Foster City, Calif., USA) according to manufacturer's protocols. Reactions were initially incubated at 25° C. for 10 minutes and subsequently at 37° C. for 120 minutes. Quantitative RT-PCR (Taqman) assays were chosen for the transcripts to be evaluated from Assays-On-Demand™ (Applied Biosystems Inc.), a pre-validated library of human specific QPCR assays, and incorporated into a 384-well Micro-Fluidics Cards. Two micro liters of the cDNA samples (diluted to 50 μl) along with 50 μl of 2×PCR master mix were loaded into respective channels on the microfludic cards followed by centrifugation. The card was then sealed and real-time PCR and relative quantification was carried out on the ABI PRISM 7900 Sequence Detection System (Applied Biosystems Inc.). All failed (undetermined) reactions were excluded and ΔCt values were calculated. For calculation of relative fold change values, initial normalization was achieved against endogenous 18S ribosomal RNA using the ΔΔCT method of quantification (Applied Biosystems Inc.) (Livak and Schmittgen, 2001). Average fold changes from four independent runs were calculated as 2^{-ΔΔCT} Significance was determined by running a 2-way ANOVA and Tukey's Pair-Wise (SAS) comparisons for each gene looking at the effects of BMP4, Noggin, KITL and MEF ECM differentiation.

### Example 7

### Loss of KIT Ligand Causes Decreased Germ Cell Gene Expression and DDX4+ POU5F1+ Cells

We demonstrated in Examples 1-4 that an enriched population of DDX4+ POU5F1+ cells can be generated from hESCs. DDX4 is a germ cell specific marker in mice (Castrillon, et al., 2000, Toyooka, et al., 2000) and humans (Castrillon, et al, 2000), while POU5F1 is a marker expressed in pluripotent cell types including germ cells (Gaskell, et al., 2004, James Kehlerl, 2004, Kerr, et al., 2008) and that feeder cells can play a significant role in differentiation. In this experiment we demonstrate that factors associated with feeder cells have a role in GLC formation. We differentiated BGO1 (XY) hESCs with a normal karyotype that were derived from a discard embryo (Mitalipova, et al., 2003). hESCs were differentiated on feeders in 20% KSR media without passaging for 10 days. Media was replenished every other day to encourage germ cell signaling. Immunocytochemistry demonstrated that number of hESCs (DDX4− POU5F1+ (FIGS. 7B and C)) were reduced after differentiation, while a significant number of POU5F1+ cells expressed the germ cell specific marker DDX4+ (FIGS. 7F and G). DDX4 and POU5F1 expression was specific as co-cultured feeder cells (FIGS. 7J and K) and human neural progenitor cells (hNPCs (FIGS. 7N and 0)) derived from hESC were consistently negative. These results were further supported by flow cytometry where hESCs (FIG. 7D) had a small subset of POU5F1+ cells that were DDX4+, while large numbers of POU5F1+ cells were also DDX4+ (FIG. 7H) in day 10 differentiation cultures. Once again, hNPCs and feeders were negative for POU5F1 and DDX4 (FIG. 6L and P, respectively) expression. This level of differentiation was achieved without the addition of exogenous signaling factors, but still undefined conditions. One potential components in these undefined conditions is feeder derived KITL, which has been proven to be essential to maintaining normal germ cell development in the mouse (Matsui, et al., 1990, Runyan, et al., 2006, Chabot, et al., 1988, Geissler, et al., 1988, Mahakali Zama, et al., 2005). To determine the effect of KITL, we used Kitl^{sl-gb} mutant mice previously demonstrated to have a complete loss of Kitl mRNA expression (Rajaraman, et al., 2002) and primordial germ cell formation in the mouse embryo to create feeders (Mahakali Zama, et al., 2005). hESCs were differentiated on Kitl +/+, +/− and −/− feeders in 20% KSR media without passaging and with media changes every other day for 10 days. Heterozygous and homozygous null expression of KITL resulted in significant reduction (p<0.05) in the KITL receptor KIT, the migratory genes CXCR4 and DDX4, the post-migratory gene DAZL and the meiotic gene SYCP3 expression relative to cultures differentiated on KITL +/+ feeders (FIG. 8A). However, differentiation on KITL −/− feeders had no significant effect on the specifying genes IFITM3 or DPPA3 the pre-migratory gene POU5F1, the migratory gene NANOG or the post-migratory genes PIWIL2, PUM2 and NANOS1 relative to samples differentiated on KITL +/+ feeders (data not shown). The percentage of DDX4+ POU5F1+ cells was also significantly (p<0.05) decreased when differentiated on KITL +/− (21.5%) and KITL −/− (10.9%) in a dose dependent manner, relative to hESCs differentiated on KITL +/+ feeders (37.0%) (FIG.

8B). This suggests that KITL is essential for enrichment of hESC to DDX4+ POU5F1+ cells.

### Example 8

### BMP Signaling Causes Increased Germ Cell Gene Expression and Germ-Like Cell Enrichment

BMP signaling activity, which is important for germ cell specification and differentiation (Lawson, et al., 1999, Ying and Zhao, 2001, Ying, et al., 2000), has been found to be present in similar culture systems, attributed to the presents of KSR (Xu, et al., 2005) and feeder cells (Qi, et al., 2004). Knowing this, we examined whether noggin, a BMP antagonist, could inhibit germ-like cell formation in our culture system. hESCs were differentiated under standard conditions in the presence of 100 ng/ml of noggin for 10 days. Under these conditions up regulation of germ cell gene expression was significantly (p<0.05) inhibited with expression levels resembling a hESC like state. The specifying gene IFITM3, the pre-migratory gene POU5F1, the migratory gene NANOG, the post-migratory gene PUM2 and the meiotic gene MLH1 all remained down regulated with the addition of noggin (FIG. 9A). DPPA3, KIT, DDX4 (FIG. 8A), DAZL, PIWIL2, NANOS1, SYCP1, SYCP3 and CXCR4 (data not shown) expression was not significantly changed relative to differentiated control (treatment without noggin or BMP4) (FIG. 9A). This indicates that BMP activity in these conditions may play a significant role in germ cell differentiation from hESCs.

To explore the ability of exogenous BMP4 in potentiating germ cell differentiation, hESCs cultures were differentiated as described above except they were exposed to 10 or 100 ng/ml of the germ cell specifying signaling molecule BMP4 for 3 days and further differentiated as before for an additional 7 days. IFITM3 gene expression was significantly increased (p<0.05) in cultures with BMP4 relative to hESCs, however this increase was not significant relative to control treatment without BMP4 (FIG. 9A). Conversely, cultures treated with BMP4 exhibited a dose dependent increase (p<0.05) in expression of the specifying gene DPPA3 and the pre-migratory genes POU5F1 and KIT relative to differentiated control cells (FIG. 9A). BMP4 also significantly increased (p<0.05) the expression of the migratory gene NANOG (FIG. 9A), the post-migratory gene DAZL (data not shown), the spermatogonia gene PUM2 and the meiotic gene MLH1 relative to hESCs and control (FIG. 9A). The migratory gene DDX4 (FIG. 9A) and the post-migratory genes PIWIL2, NANOS1, SYCP3 (data not shown) showed no significant change with the addition of BMP4, however they were all more highly expressed in the treated cells relative to hESCs. There was no change in expression of the migratory gene CXCR4 and the meiotic gene SYCP1 (data not shown) relative to hESCs or control treatment. The significant effect of additional BMP4 in 7 genes suggest that BMP4 further enriched germ-like character in these cultures.

To further confirm the role of BMP4 in germ-like cell enrichment, flow cytometry was conducted to quantify the population of DDX4+ POU5F1+ cells. The addition of noggin significantly (p<0.05) inhibited the enrichment of germ-like cells with only 5.9% of cells being DDX4+ POU5F1+, a percentage not significantly (p<0.05) different from hESCs (4.1%) (FIG. 9B). Additional exogenous BMP4 did not significantly (p<0.05) increase the percentage of germ-like cells, 29.6% of cells being DDX4+ POU5F1+, relative to control treatment (FIG. 8B). This suggests that supplemental BMP4 levels in excess of the BMP activity in KSR or produced by feeders is not required for the formation of DDX4+ POU5F1+ cells, albeit the addition of BMP4 causes increased germ cell gene expression and potentially enhanced germ cell programming.

### Example 9

### Differentiation on Feeder Extracellular Matrix Causes Decreased Germ Cell Gene Expression

The experiments of Examples 1-4 showed that differentiation of hESCs into germ-like cells requires cell-cell contact with feeders. Differentiation in feeder conditioned media, media exposed to feeders for 24 hrs, on poly-ornithine and laminin coated plates caused a significant reduction in germ cell gene expression and, in the absence of bFGF, DDX4+ POU5F1+ cells. To determine if feeder ECM promotes germ-like cell differentiation, hESCs were differentiated in 20% KSR media on feeder ECM with every other day media changes and no passaging for 10 days. Differentiation of hESCs on feeder ECM significantly reduced (p<0.05) pre-migratory genes IFITM3, DPPA3 and KIT, the migratory gene NANOG, the post-migratory gene PUM2 and the meiotic genes MLH1 and SYCP3, relative to control cells differentiated on feeders (FIG. 10A). There was no significant change in the pre-migratory genes POU5F1, the migratory gene DDX4, the post-Migratory genes DAZL, PIWIL2, NANOS1 and the meiotic gene SYCP1 with respect to hESCs differentiated on feeders (FIG. 10A). The down regulation of 7 germ cell genes represents a decrease in germ cell character suggesting that direct communication between the feeders and hESCs is important for the enhancement of germ cell gene expression. Despite the down regulation of germ cell genes in cultures differentiated on feeder ECM, this condition did not significantly affect the number of DDX4+ POU5F1+ germ-like cells 34.2% on ECM vs. 30.9% on feeders (FIG. 10B). This suggests that germ-like cells can be derived on the feeder ECM alone, however differentiation on feeders results in higher germ cell gene expression.

### Discussion of the Experimental Results of Examples 7-9

The experiments of Examples 7-9 show for the first time the role of KITL in the in vitro differentiation of hESCs into germ cells. When KITL was absent, there was a significant decrease in DDX4+ POU5F1+ cells (FIG. 8B). Previously, KITL signaling has been shown to play a pivotal role in promoting proliferation of mouse prenatal germ cells (Runyan, et al., 2006, Chabot, et al., 1988) and is believed to do the same in human germ cell development (Hoyer, et al., 2005, Robinson, et al., 2001). KITL is also essential in maintaining extended mouse primary germ cell cultures (Godin, et al., 1991, Matsui, et al., 1991, Resnick, et al., 1992, Pesce, et al., 1993, Pesce, et al., 1997). Matsu et al. (Matsui, et al., 1991) showed that primary mouse germ cell cultures grown on KITL −/− feeders had a significant reduction in proliferation and survivability. Even in the absence of feeders, which are known to produce other factors important for primary germ cell culture like leukemia inhibitory factory (Matsui, et al., 1991), KITL is able to enhance initial survival of primary germ cells in culture (Godin, et al., 1991). Here the proliferative response of hESC derived germ-like cells to KITL correlates with cultures of primary germ cells and implies that these may model human germ cell development.

KITL/KIT signaling is intrinsically linked to the migratory phase of early development as KIT is first expressed in mouse primordial germ cells (PGCs) at 7.5 days post coitum (dpc) (Manova and Bachvarova, 1991), just proceeding initiation of migration, and KITL is expressed throughout the migratory pathway and at the genital ridge (Matsui, et al., 1990). KITL/KIT signaling is believed to play an important role in germ cell development during the migratory phase (Runyan, et al., 2006). This may explain why we found a significant decrease in expression of CXCR4, DDX4, both are first expressed during migration, in cells on KITL −/− feeders (FIG. 8A). KITL/KIT signaling is also essential in postnatal germ cell development and interrupting this signaling leads to a loss of A spermatogonia derivatives, including meiotic cell types (Yoshinaga, et al., 1991, Manova, et al., 1993). In agreement, we observed decreased expression of SYCP3, an early meiotic marker (Yuan, et al., 2002, Yuan, et al., 2000), in the KITL deficient groups (FIG. 8). In addition, germ cells just preceding entry into meiosis express KIT and are believed to be responsive to KITL/KIT signaling (Yoshinaga, et al., 1991, Manova, et al., 1993). Conceivably, SYCP3 gene expression may also be linked to KITL/KIT signaling, yet further studies are needed to confirm. We did not observe a significant change in the expression of specifying genes IFITM3 or DPPA3, yet we did see a significant decrease in the number of germ-like cells. We hypothesize that the absence of KITL does not affect the specification of germ-like cells, however it may prevent expansion of these cells. A possible loss in expansion is in agreement with data in the mouse where early mouse germ cells show a loss of proliferation and undergo apoptosis when KITL/KIT signaling is interrupted (Runyan, et al., 2006, Chabot, et al., 1988, Manova and Bachvarova, 1991). Overall, our finding agree with previous in vivo and in vitro germ cell culture data and suggests KITL plays a key role in proliferation and differentiation of these hESC derived germ-like cells.

Noggin's inhibition of BMP signaling in differentiating cultures decreased germ cell gene expression and all but eliminated differentiation to DDX4+ POU5F1+ cells. Previous studies differentiating ESCs into germ cells used undefined culture condition including fetal bovine serum (FBS) or KSR which may have affected differentiation via BMP activity (Hubner, et al., 2003, West, et al., 2008, Xu, et al., 2005, Chen, et al., 2007, Clark, et al., 2004). These results suggest that the inherent BMP activity of these systems is sufficient to cause differentiation in to germ-like cells. BMP4 has also been shown to be produced by MEF feeders (Qi, et al., 2004) and may be a major source of germ cell signaling in an adherent culture system. This may explain the relatively high numbers of germ-like cells observed in this culture system and provides a significant advantage over embyroid body differentiation cultures that do not utilize feeders (Clark, et al., 2004, Kee, et al., 2006). As anticipated based on BMP4's role in specification in vivo (Lawson, et al., 1999, Ying, et al., 2001, Ying and Zhao, 2001, de Sousa Lopes, et al., 2004), adding BMP4 caused an increase in germ cell specifying genes. Toyooka et al. (Toyooka, et al., 2003) also showed that BMP4 produced DDX4+ cells from mESCs, the mouse homologue of DDX4; however, our cultures already contained DDX4+ cells, making it hard to directly compare these two studies. In addition, here BMP4 caused an increase in migratory and post-migratory gene expression, which has not been previously show and may represent enhanced germ cell programming.

Kee et al. (Kee, et al., 2006) also used exogenous BMP4 in the differentiation of hESCs into germ-like cells. They showed that BMP4 induced a 3.4 fold increase in DDX4 gene expression when compared to differentiated treatments without BMP4. Also, BMP4 caused a significant increase in the DDX4+ population from 3% to 14.5% with the inclusion of additional germ cell enhancing factors BMP7 and BMP8b (Kee, et al., 2006). We observed a similar fold increase of 3.8 in DDX4 gene expression when 10 ng/ml of BMP4 was added (FIG. 9A). Although this was not a statistically significant increase, it may be biologically significant. Further, we did not see a significant increase in the number of DDX4+ POU5F1+ cells in the presence of exogenous BMP4 (FIG. 9B). Again, the addition of noggin into our non-supplemented cultures reduced germ cell gene expression and DDX4+ POU5F1+ cells, implying that these cultures have endogenous BMP activity (FIG. 9A). Therefore the addition of exogenous BMP4 beyond that present in KSR or produced by feeders may be redundant and may not further enhance germ-like cell production.

Despite the down regulation of germ cell genes in cultures differentiated on feeder ECM (FIG. 10A), the proportion of DDX4+ POU5F1+ cells remained the same as feeder cultured cells (FIG. 10B). This result is not totally unexpected since neither POU5F1 nor DDX4 gene expression was significantly changed in cultures differentiated on ECM. The specifying genes IFITM3 and DPPA3 and the post-migratory genes PUM2, MLH1 and SYCP3 all showed significant decreased expression relative to cells differentiated on feeder cells. However, these same genes were up regulated relative to hESCs, thus the relatively lower germ cell gene expression in feeder ECM vs the feeder cell groups may represent only a partial failure to recapitulate the germ cell/feeder cell association.

In summary, the experiments of Examples 7-9 are the first report demonstrating that a loss of KITL in differentiation cultures caused a significant decrease in enrichment of human germ-like cells and germ cell genes that are temporally correlated. BMP4 caused a significant increase in germ cell gene expression and appears to be required for differentiation of hESCs into germ-like (DDX4+ POU5F1+) cells. Additionally, other factors and conditions are likely required for proper temporal and spatial germ cell development events to be mimicked in vitro. Building upon these studies, future work will investigate the role of different factors, germ cell niche signaling and comparing germ cell programming of hESC derived germ cells to their in vivo counterparts.

### Part 3

### Overview

STRA8 is believed to be a key factor in initiating the transition from mitotic to meiotic germ cells in both female and male germ cell development [19, 20] Timing of meiosis in the mouse is sex specific with initiation of meiosis in response to retinoic acid (RA) signaling occurring in female mice during embryonic development and in male counterparts soon after birth. STRA8 expression in the embryonic ovary occurs in an anterior to posterior wave followed by a wave of meiotic gene expression [31, 32]. STRA8 is expressed in the mitotic germ cells of the testis as well as the preleptotene, the most advanced cell type before meiotic prophase [33, 34]. After initiation of meiosis by STRA8 expression in wild-type mice, germ cells enter into meiotic S-phase, where DNA is replicated [22]. During this stage cohesion occurs between chromosomes by formation of a cohesion complex composed of meiotic specific proteins REC8 [1, 35] and SMC113 [2, 36]. Germ cells then advance to the first stage of meiosis, prophase I, which is composed of 5 phases: leptotene, zygotene, pachytene, diplotene and diakinesis. It is during leptotene that double stranded breaks (DSBs) are initially formed by the enzyme Spo11 [11]. These DSBs facilitate crossing over events and the formation chiasmata that are essential for genetic exchange. DSBs are also marked by the phosphorylated H2AX, γ-H2AX, which is believed to function as a recruiting mechanism for recombinant proteins to DSBs [12, 22]. Chromosomes are soon aligned by lateral elements formed by synaptonemal complex proteins (SYCP) 2 and SYCP3 and connected by transverse filaments composed of SYCP1 [3, 8, 9]. The synaptonemal complex formed by these proteins are essential to synapsis, the pairing of homologous chromosomes during zygotene, with aberrant synapsis leading to sterility [8, 9]. The pachytene and diplotente stages are marked by DNA recombination where genetic information is exchanged between overlapping chromatids. This is also where repair of DSBs begins with aid of DNA repair proteins DMC1 and MLH1 [22, 37, 38]. The expression of STRA is essential for these early meiotic events to occur with loss of expression resulting in arrest in germ cell development and sterility.

STRA8 deficient mice show significant deviation from normal early meiotic activity. Both sexes demonstrate a loss of DNA condensation indicative of the leptotene stage of prophase and show no indication of advancement into the later stages of zygotene or pachytene [19, 20]. Proteins comprising cohesion and synaptonemal complexes which are normally closely associated with chromosomes show lack of localization and diffused expression throughout the nucleus [19, 20]. In addition STRA8 homozygous knockout mice lack DSBs indicated by the absence of the modified histone γ-H2AX [12], low gene expression levels of the DSB forming enzyme Spoil [11] and the DSB repair gene Dmc1 [37]. The lack of DSBs also suggests an inability to undergo genetic recombination. The sum of these data indicate that STRA8 plays a significant role in the initiation of meiosis with its lack of expression resulting in the loss of advanced meiotic phenotypes, inhibition of cohesion and synapsis, genetic recombination, and most importantly, fertility.

### Example 10

### Meiotic Activity in Germ-Like Cell Cultures

Meiosis is a key step in germ cell development and an indicator of germ cell formation in vitro. In order to ascertain whether cells in DDX4+ POU5F1+ cultures obtained as described in Examples 1-4 were undergoing meiosis, quantitative (q)RT-PCR was performed to detect gene expression levels of the meiotic markers MLH1, a protein essential for meiotic chiasmata formation [65, 66], and SCP3, a protein involved in the formation of synaptonemal complexes in meiosis [8-10]. SCP3 and MLH1 gene expression showed a statistically significant variation over the course of the experiment with increased expression at days 10 and 30, compared to Day 3 (FIGS. 10A and B). A similar trend was also seen for increased DDX4 and POU5F1 gene expression at Days 10 and 30 (FIGS. 10C and D). To further confirm whether germ-like cells enter meiosis in culture, hESCs were differentiated for 10, 16, and 30 days on feeders in the presence of bFGF and immunostained for MLH1 and SYCP3. Immunostaining showed that >90% of day 16 cells were positive for MLH1 (FIGS. 11E and 11F) and SYCP3 (FIGS. 11G and 11H) proteins, whereas no expression of either marker was found in hESCs (FIG. 11A-11D), day 10 (data not shown), or day 30 cells. Staining was localized to the nucleus, which correlates with their known role in chromosome segregation during meiosis [8, 9, 26, 65]. However, these proteins did not show further progression with the complete formation of synaptonemal complexes. In fact, the localizations seemed to be consistent with what had been previously reported in the case of STRA8 mutants, suggesting abnormal meiotic initiation [19, 20]. STRA8 and CYP26b1, the factor that inhibits STRA8 expression in vivo, gene expression was examined at day 10 and 16 by RT-PCR, however expression of neither molecule was found, possibly due to the lack of R signaling in our cultures.

### Example 11

### Continual Expansion and Transduction of Germ-Like Cells

One disadvantage of cultured germ cells is the inability to easily maintain these lines as they have a high propensity to senesce or differentiate into unwanted cell types [67]. However, we accomplished this difficult task. After differentiation of hESCs for 10 days, we have found that we can maintain and further enrich GLCs (DDX4+ POU5F1+) for at least 20 passages (FIG. 12A), thus allowing for continual expansion of these cells for future studies. This also provides an opportunity to produce a clonal population of hESC derived GLCS, which has not been previously done. This is important as the current system produces mixed cell populations of varying lineages and at different stages of germ cell development [23]. Potentially GLCs can be transduced and undergo fluorescence activated cell sorting (FACS) for clonal expansion. As these cells are similar to hESCs, which are notoriously difficult to transduce, there is significant concern with respect to transducibility. After differentiation of hESCs for 10 days under standard conditions, cells were transduced using a lentiviral GFP reporter system with GeneJammer, a reagent that increases transduction efficiency in stem cells [68, 69]. GLC cultures showed a high propensity to undergo transduction with >90% cells showing GFP expression (FIGS. 12B and C). These cells have maintained high level of expression for 5 passages, with no notable loss in expression (silencing). This suggests that these cells can be clonally expanded and give rise to a homogeneous population of GLCs, providing a robust assay system that was previously unavailable. Given this recent significant advance in our lab we then progressed toward clonal isolation of these cells.

### Example 12

### Isolation and Meiotic Differentiation of Clonal Germ-Like Cells

Mixed cultures have hampered the advancement of the hESC field by inhibiting the ability to determine a cell line's potency and potential to uniformly differentiate to a desired cell type. Throughout the literature and in our own hands, high variability has been noted between derived GLCs in response to treatments. Contaminating cell types are found in our differentiation cultures and variability is observed from derivation to derivation. We sought to address these issues by deriving clonal GLC lines that would be highly enriched for DDX4+ POU5F1+ cells and phenotypically identical, eliminating the issue of derivation variability. GFP+ GLCs underwent fluorescence activated cell sorting (FACS) where a single cell was plated into a well of a 96 well plate containing feeders and 20% KSR media plus 10 μM of Y-27632, a selective Rho-associated kinase (ROCK) inhibitor that improves survival of disassociated stem cells by inhibiting apoptosis [70]. Clonal GLCs were expanded producing 39 lines of which 5 were selected for experimental use. Flow cytometry confirmed the GLC identity of these cell lines. Out of the 5 clonal lines, 3 lines (Clones 1-3) possessed >90% DDX4 POU5F1 expression (FIG. 13A), while the remaining 2 lines (Clones 4-5) were only POU5F1+ and believed to be hESCs (FIG. 13B). Clonal GLCs lines were differentiated into meiotic GLCs, samples were differentiated for 0, 6 and 10 days and then analyzed by flow for SYCP3 and MLH1 expression. Clones 1-3 were positive for both SYCP3 (75.4%-88% positive FIG. 13C) and MLH1 (80.6%-87.6% positive FIG. 13E) at day 10 and negative at day 0 and 6 (data not shown). Clones 4 and 5 produced <4% DDX4+ cells for either marker at days 0, 6 (data not shown) and 10 (SYCP3—FIG. 14D; MLH1—FIG. 13F).

To our knowledge, this represents the first time that a pure population of hESCs derived GLCs has been created.

Induced pluripotent stem cells (iPSCs) derived from IMR90 lung fibroblast cells have also exhibited the ability to differentiate into GLCs utilizing the 2-D culture system previously described. This indicates that patient specific GLCs can be created using this system.

### Example 13

### Prophetic

A TZV family STRA8 vector containing CMV promoter driven puromycin resistance and a tetracycline responsive element (TRE)-CMV promoter driven STRA8 is prepared as shown in FIG. 15.

### Sample Transduction

A clonal population of XY GLCs (obtained as described in Examples 8-10) plated at density of 1,500 cells/mm² undergoes lentiviral transduction with the designed tet-on vector at a multiplicity of infection (MOI) of 20, based on preliminary studies, and GeneJammer. Cells undergo expansion and are maintained under puromycin selection, optimum concentration based on a validated kill curve, for a minimum of 2 weeks and throughout experiments to create a pure population and to remove all cells that undergo gene silencing. This will also confirm the presence of the integrated construct.

To determine a low, medium and high level of doxycycline induced STRA8 expression, transduced GLCs are exposed to 0, 3, 10, 30, 100, 300 and 1000 ng/ml of doxycycline for 0, 24, 48 and 72 hrs to determine levels of STRA8 expression. STRA8

expression is confirmed and quantified at the gene and protein levels by qRT-PCR and western blot respectively. To ascertain the effectiveness of puromycin selection of STRA8 expressing cells, flow cytometry is performed to determine the percentage of STRA8+ cells.

### Representative Statistical Analysis

Overall treatment effects of doxycycline and time of exposure are determined by analysis of variance (ANOVA) with specific treatment effects being determined by Tukey pair-wise comparisons with all possible pairs being taken into account. Treatments with a p<0.05 are considered significant.

Since similar lentiviral vectors are used in the GLC culture, a high proportion of vector positive cultures is anticipated. Additionally, puromycin selection has been used on hESC and derivatives by numerous labs (Dhara et al., 2009) and it should be possible to enrich for STRA8 vector positive cells. Doxycycline exposure from 0 to 1000 ng/ml resultd in a significant and correlative increase between doxycycline and STRA8 gene and protein expression as determined by qRT-PCR and western blot analysis. Increases in STRA8 expression occur over time and the highest levels of mRNA and protein in 1000 ng/ml doxycycline are observed at 72 hrs. Flow cytometry results show all treatments receiving doxycyline express STRA8 in >95% of GLCs, while treatments without doxycycline express STRA8 in <5% of cells.

### Example 14

### Prophetic

### Over Expression of STRA8 Activates Meiotic Cohesion, the Development and Repair of DSBs, Synapsis and Genetic Recombination

GLCs are exposed to low, medium and high levels of STRA8 expression based on doxycycline concentrations proposed in Example 12 (Prophetic) for 0, 12, 24, 48 and 72 hrs. Cells are analyzed by quantitative RT-PCR for the expression of the meiotic specific cohesion proteins REC8 [1, 35] and SMC1β [2, 36], synaptonemal complexes proteins SYCP3 [8, 9], SYCP1 [10, 71] and three genes involved the formation and repair of DSBs, SPO11, DMC1 [37] and MLH1 [65, 72] with the doxycyline dose with the highest levels of gene expression being considered optimum. Transduced cells lacking the Stra8 gene are used as a control cell type.

Based on timing and optimum doxycycline induced STRA8 meiotic gene expression, immunocytochemistry is performed to determine appropriate formation of cohesion and synaptomenal complexes, localization of SPO11, DMC1 and MLH1 proteins and the DSB marker γ-H2AX. Analyzing proteins for cohesion, synapsis and DSB formation and repair by flow cytometry develops a profile demonstrating order, timing and the number of cells undergoing each process. To further validate genetic recombination, transmission electron microscopy (TEM) is performed to look for chiasmata formation in meiotic cells. All experiments are replicated a minimum of three times and statistical analysis is conducted as proposed in Example 13 (Prophetic).

The highest level of STRA8 over expression results in the highest (and believed to be optimum) levels of meiotic cohesion, synapsis and DSB formation and repair, relative to STRA8-cells. Additionally, these cells demonstrate appropriate protein localization and complex formation indicative of these processes. Flow cytometry results show an increase in meiotic protein expression in STRA8+ GLCs, relative to STRA8− controls, and that GLCs undergo ordered formation transitioning into meiotic cohesion, DSB formation, synapsis and then DSB repair post-Stra8 activation. In normal development, the formation of chiasmata should be observed following synapsis and proceeding DSB repair and is expected to be recapitulated in hESC derived GLCs and confirmed by TEM

### Example 15

### Prophetic

### STRA8 Expression Results in Completion of Meiosis and the Formation of Haploid Germ-Like Cells

### Experimental Design

XY GLCs are stained with the fluorescent DNA binding molecule 4,6-diamidino-2-phenylindole (DAPI) at day 0, 10 and 20 after initiation of STRA8 expression and are analyzed by flow cytometry. Diploid cells normally emit light at a set level of relative florescence intensity (RFI) after DAPI staining and a 50% decrease in RFI indicates that cells have undergone meiosis and exist in a haploid state [50, 51]. Cytogenetic analysis of treatments demonstrating decreased DNA content by karyotyping is done to further confirm the haploid state. Porcine haploid gametes, as they are easy to obtain, are used as a positive assay control and all experiments undergo a minimum of three replications.

A sub-set of STRA8 expressing germ cells completes meiosis indicated by a 50% decrease in RFI by flow cytometry within 10 to 20 days. Additional cytogenetic analysis confirms the presence of only 23 chromosomes in a population of post-meiotic GLCs.

### REFERENCE COLLECTION 1

1 Stephen E H, Chandra A. Declining estimates of infertility in the United States: 1982-2002. Fertil Steril 2006; 86:516-523.
2 Fujita K, Tsujimura A, Takao T et al. Expression of inhibin alpha, glial cell line-derived neurotrophic factor and stem cell factor in Sertoli cell-only syndrome: Relation to successful sperm retrieval by microdissection testicular sperm extraction. Hum Reprod 2005; 20:2289-2294.
3 Clark A T, Bodnar M S, Fox M et al. Spontaneous differentiation of germ cells from human embryonic stem cells in vitro. Hum Mol Genet. 2004; 13:727-739.
4 Hubner K, Fuhrmann G, Christenson L K et al. Derivation of oocytes from mouse embryonic stem cells. Science 2003; 300:1251-1256.
5 Toyooka Y, Tsunekawa N, Akasu R et al. Embryonic stem cells can form germ cells in vitro. Proc Natl Acad Sci USA 2003; 100:11457-11462.
6 Gaskell T L, Esnal A, Robinson L L et al. Immunohistochemical profiling of germ cells within the human fetal testis: Identification of three subpopulations. Biol Reprod 2004; 71:2012-2021.
7 Saitou M, Payer B, Lange U C et al. Specification of germ cell fate in mice. Philos Trans R Soc Lond B Biol Sci 2003; 358:1363-1370.
8 Lawson K A, Dunn N R, Roelen B A et al. Bmp4 is required for the generation of primordial germ cells in the mouse embryo. Genes Dev 1999; 13:424-436.
9 Ying Y, Qi X, Zhao G Q. Induction of primordial germ cells from murine epiblasts by synergistic action of BMP4 and BMP8B signaling pathways. Proc Natl Acad Sci USA 2001; 98:7858-7862.

Castrillon D H, Quade B J, Wang T Y et al. The human VASA gene is specifically expressed in the germ cell lineage. Proc Natl Acad Sci USA 2000; 97:9585-9590.
11 Hay B, Jan L Y, Jan Y N. A protein component of Drosophila polar granules is encoded by vasa and has extensive sequence similarity to ATP-dependent helicases. Cell 1988; 55:577-587.
12 Tanaka S S, Toyooka Y, Akasu R et al. The mouse homolog of Drosophila Vasa is required for the development of male germ cells. Genes Dev 2000; 14:841-853.
13 Ikenishi K, Tanaka T S. Involvement of the protein of Xenopus vasa homolog (Xenopus vasa-like gene 1, XVLG1) in the differentiation of primordial germ cells. Dev Growth Differ 1997; 39:625-633.
14 Kee K, Gonsalves J M, Clark A T et al. Bone morphogenetic proteins induce germ cell differentiation from human embryonic stem cells. Stem Cells Dev 2006; 15:831-837.
15 Nakatsuji N, Chuma S. Differentiation of mouse primordial germ cells into female or male germ cells. Int J Dev Biol 2001; 45:541-548.
16 Yuan L, Liu J G, Zhao J et al. The murine SCP3 gene is required for synaptonemal complex assembly, chromosome synapsis, and male fertility. Mol Cell 2000; 5:73-83.
17 Yuan L, Liu J G, Hoja M R et al. Female germ cell aneuploidy and embryo death in mice lacking the meiosis-specific protein SCP3. Science 2002; 296:1115-1118.
18 Di Carlo A D, Travia G, De Felici M. The meiotic specific synaptonemal complex protein SCP3 is expressed by female and male primordial germ cells of the mouse embryo. Int J Dev Biol 2000; 44:241-244.
19 Anderson L K, Reeves A, Webb L M et al. Distribution of crossing over on mouse synaptonemal complexes using immunofluorescent localization of MLH1 protein. Genetics 1999; 151:1569-1579.
20. Edelmann W, Cohen P E, Kane M et al. Meiotic pachytene arrest in MLH1-deficient mice. Cell 1996; 85:1125-1134.
21 Geijsen N, Horoschak M, Kim K et al. Derivation of embryonic germ cells and male gametes from embryonic stem cells. Nature 2004; 427: 148-154.
22 Nayernia K, Nolte J, Michelmann H W et al. In vitro-differentiated embryonic stem cells give rise to male gametes that can generate offspring mice. Dev Cell 2006; 11:125-132.
23 Resnick J L, Ortiz M, Keller J R et al. Role of fibroblast growth factors and their receptors in mouse primordial germ cell growth. Biol Reprod 1998; 59:1224-1229.
24 Resnick J L, Bixler L S, Cheng L et al. Long-term proliferation of mouse primordial germ cells in culture. Nature 1992; 359:550-551.
25 Matsui Y, Toksoz D, Nishikawa S et al. Effect of Steel factor and leukaemia inhibitory factor on murine primordial germ cells in culture. Nature 1991; 353:750-752.
26 Mitalipova M, Calhoun J, Shin S et al. Human embryonic stem cell lines derived from discarded embryos. STEM CELLS 2003; 21:521-526.
27 Livak K J, Schmittgen T D. Analysis of relative gene expression data using real-time quantitative PCR and the 2(-Delta Delta C(T)) method. Methods 2001; 25:402-408.
28 Lacham-Kaplan O, Chy H, Trounson A. Testicular cell conditioned medium supports differentiation of embryonic stem cells into ovarian structures containing oocytes. STEM CELLS 2006; 24:266-273.
29 Bowles J, Knight D, Smith C et al. Retinoid signaling determines germ cell fate in mice. Science 2006; 312:596-600.
Koubova J, Menke D B, Zhou Q et al. Retinoic acid regulates sex-specific timing of meiotic initiation in mice. Proc Natl Acad Sci USA 2006; 103:2474-2479.
31 Codina-Pascual M, Oliver-Bonet M, Navarro J et al. Synapsis and meiotic recombination analyses: MLH1 focus in the XY pair as an indicator. Hum Reprod 2005; 20:2133-2139.
32 Chen H F, Kuo H C, Chien C L et al. Derivation, characterization and differentiation of human embryonic stem cells: Comparing serum-containing versus serum-free media and evidence of germ cell differentiation. Hum Reprod 2007; 22:567-577.
33 Thomson J A, Itskovitz-Eldor J, Shapiro S S et al. Embryonic stem cell lines derived from human blastocysts. Science 1998; 282:1145-1147.34 Dvorak P, Hampl A. Basic fibroblast growth factor and its receptors in human embryonic stem cells. Folia Histochem Cytobiol 2005; 43: 203-208.
35 Sette C, Dolci S, Geremia R et al. The role of stem cell factor and of alternative c-kit gene products in the establishment, maintenance and function of germ cells. Int J Dev Biol 2000; 44:599-608.
36 Xu R H, Peck R M, Li D S et al. Basic FGF and suppression of BMP signaling sustain undifferentiated proliferation of human ES cells. Nat Methods 2005; 2:185-190.
37 Ying Y, Zhao G Q. Cooperation of endoderm-derived BMP2 and extraembryonic ectoderm-derived BMP4 in primordial germ cell generation in the mouse. Dev Biol 2001; 232:484-492.
38 Tribulo C, Aybar M J, Nguyen V H et al. Regulation of Msx genes by a Bmp gradient is essential for neural crest specification. Development 2003; 130:6441-6452.
39 Xu R H, Chen X, Li D S et al. BMP4 initiates human embryonic stem cell differentiation to trophoblast. Nat Biotechnol 2002; 20:1261-1264.
40 Turnpenny L, Spalluto C M, Perrett R M et al. Evaluating human embryonic germ cells: Concord and conflict as pluripotent stem cells. STEM CELLS 2006; 24:212-220.
41 Toyooka Y, Tsunekawa N, Takahashi Y et al. Expression and intracellular localization of mouse Vasa-homologue protein during germ cell development. Mech Dev 2000; 93:139-149.
42 Sekiguchi T, Hayano T, Yanagida M et al. NOP132 is required for proper nucleolus localization of DEAD-box RNA helicase DDX47. Nucleic Acids Res 2006; 34:4593-4608.
43 Chambers I, Colby D, Robertson M et al. Functional expression cloning of Nanog, a pluripotency sustaining factor in embryonic stem cells. Cell 2003; 113:643-655.
44 Pesce M, Scholer H R. Oct-4: Gatekeeper in the beginnings of mammalian development. STEM CELLS 2001; 19:271-278.
45 McLaren A, Southee D. Entry of mouse embryonic germ cells into meiosis. Dev Biol 1997; 187:107-113.
46 Zamboni L, Upadhyay S. Germ cell differentiation in mouse adrenal glands. J Exp Zool 1983; 228:173

### REFERENCE COLLECTION 2

Geijsen, N., Horoschak, M., Kim, K., Gribnau, J., Eggan, K. and Daley, G. Q. (2004) Derivation of embryonic germ cells and male gametes from embryonic stem cells. Nature, 427, 148-154.
Hubner, K., Fuhrmann, G., Christenson, L. K., Kehler, J., Reinbold, R., De La Fuente, R., Wood, J., Strauss, J. F., III, Boiani, M. and Scholer, H. R. (2003) Derivation of Oocytes from Mouse Embryonic Stem Cells. Science, 300, 1251-1256.
Nayernia, K., Nolte, J., Michelmann, H. W., Lee, J. H., Rathsack, K., Drusenheimer, N., Dev, A., Wulf, G., Ehrmann, I. E., Elliott, D. J. et al. (2006) In vitro-differentiated embryonic stem cells give rise to male gametes that can generate offspring mice. Dev Cell, 11, 125-132.
Toyooka, Y., Tsunekawa, N., Akasu, R. and Noce, T. (2003) Embryonic stem cells can form germ cells in vitro. Proc Natl Acad Sci USA, 100, 11457-11462.
Matsui, Y., Zsebo, K. M. and Hogan, B. L. (1990) Embryonic expression of a haematopoietic growth factor encoded by the 51 locus and the ligand for c-kit. Nature, 347, 667-669.
Lawson, K. A., Dunn, N. R., Roelen, B. A. J., Zeinstra, L. M., Davis, A. M., Wright, C. V. E., Korving, J. P. W. F. M. and Hogan, B. L. M. (1999) Bmp4 is required for the generation of primordial germ cells in the mouse embryo. Genes Dev, 13, 424-436.
Hoyer, P. E., Byskov, A. G. and Mollgard, K. (2005) Stem cell factor and c-Kit in human primordial germ cells and fetal ovaries. Mol Cell Endocrinol, 234, 1-10.
Runyan, C., Schaible, K., Molyneaux, K., Wang, Z., Levin, L. and Wylie, C. (2006) Steel factor controls midline cell death of primordial germ cells and is essential for their normal proliferation and migration. Development, 133, 4861-4869.
Chabot, B., Stephenson, D. A., Chapman, V. M., Besmer, P. and Bernstein, A. (1988) The proto-oncogene c-kit encoding a transmembrane tyrosine kinase receptor maps to the mouse W locus. Nature, 335, 88-89.
Geissler, E. N., Ryan, M. A. and Housman, D. E. (1988) The dominant-white spotting (W) locus of the mouse encodes the c-kit proto-oncogene. Cell, 55, 185-192.
Mahakali Zama, A., Hudson, F. P., III and Bedell, M. A. (2005) Analysis of Hypomorphic KitlSl Mutants Suggests Different Requirements for KITL in Proliferation and Migration of Mouse Primordial Germ Cells. Biol Reprod, 73, 639-647.
Yoshinaga, K., Nishikawa, S., Ogawa, M., Hayashi, S., Kunisada, T. and Fujimoto, T. (1991) Role of c-kit in mouse spermatogenesis: identification of spermatogonia as a specific site of c-kit expression and function. Development, 113, 689-699.
Packer, A. I., Besmer, P. and Bachvarova, R. F. (1995) Kit ligand mediates survival of type A spermatogonia and dividing spermatocytes in postnatal mouse testes. Mol Reprod Dev, 42, 303-310.
Sette, C., Dolci, S., Geremia, R. and Rossi, P. (2000) The role of stem cell factor and of alternative c-kit gene products in the establishment, maintenance and function of germ cells. Int J Dev Biol, 44, 599-608.
Manova, K., Huang, E. J., Angeles, M., De Leon, V., Sanchez, S., Pronovost, S. M., Besmer, P. and Bachvarova, R. F. (1993) The expression pattern of the c-kit ligand in gonads of mice supports a role for the c-kit receptor in oocyte growth and in proliferation of spermatogonia. Dev Biol, 157, 85-99.
Godin, I., Deed, R., Cooke, J., Zsebo, K., Dexter, M. and Wylie, C. C. (1991) Effects of the steel gene product on mouse primordial germ cells in culture. Nature, 352, 807-809.
Feng, H. L., Sandlow, J. I., Sparks, A. E., Sandra, A. and Zheng, L. J. (1999) Decreased expression of the c-kit receptor is associated with increased apoptosis in subfertile human testes. Fertil Steril, 71, 85-89.
Sandlow, J. I., Feng, H. L. and Sandra, A. (1997) Localization and expression of the c-kit receptor protein in human and rodent testis and sperm. Urology, 49, 494-500.
Sandlow, J. I., Feng, H. L., Cohen, M. B. and Sandra, A. (1996) Expression of c-KIT and its ligand, stem cell factor, in normal and subfertile human testicular tissue. J Androl, 17, 403-408.
Ying, Y., Qi, X. and Zhao, G. Q. (2001) Induction of primordial germ cells from murine epiblasts by synergistic action of BMP4 and BMP8B signaling pathways. Proc Natl Acad Sci USA, 98, 7858-7862.
Ying, Y. and Zhao, G. Q. (2001) Cooperation of endoderm-derived BMP2 and extraembryonic ectoderm-derived BMP4 in primordial germ cell generation in the mouse. Dev Biol, 232, 484-492.
Chang, H. and Matzuk, M. M. (2001) Smad5 is required for mouse primordial germ cell development. Mech Dev, 104, 61-67.
Hayashi, K., Kobayashi, T., Umino, T., Goitsuka, R., Matsui, Y. and Kitamura, D. (2002) SMAD1 signaling is critical for initial commitment of germ cell lineage from mouse epiblast. Mech Dev, 118, 99-109.
Okamura, D., Hayashi, K. and Matsui, Y. (2005) Mouse epiblasts change responsiveness to BMP4 signal required for PGC formation through functions of extraembryonic ectoderm. Mol Reprod Dev, 70, 20-29.
West, F. D., Machacek, D. W., Boyd, N. L., Pandiyan, K., Robbins, K. R. and Stice, S. L. (2008) Enrichment and differentiation of human germ-like cells mediated by feeder cells and basic fibroblast growth factor signaling. Stem Cells, 26, 2768-2776.
Kihara, T., Hirose, M., Oshima, A. and Ohgushi, H. (2006) Exogenous type I collagen facilitates osteogenic differentiation and acts as a substrate for mineralization of rat marrow mesenchymal stem cells in vitro. Biochem Biophys Res Commun, 341, 1029-1035.
Naugle, J. E., Olson, E. R., Zhang, X., Mase, S. E., Pilati, C. F., Maron, M. B., Folkesson, H. G., Horne, W. I., Doane, K. J. and Meszaros, J. G. (2006) Type VI collagen induces cardiac myofibroblast differentiation: implications for postinfarction remodeling. Am J Physiol Heart Circ Physiol, 290, H323-330.
Suzuki, A., Iwama, A., Miyashita, H., Nakauchi, H. and Taniguchi, H. (2003) Role for growth factors and extracellular matrix in controlling differentiation of prospectively isolated hepatic stem cells. Development, 130, 2513-2524.
Chen, S. S., Fitzgerald, W., Zimmerberg, J., Kleinman, H. K. and Margolis, L. (2007) Cell-cell and cell-extracellular matrix interactions regulate embryonic stem cell differentiation. Stem Cells, 25, 553-561.
Kawasaki, H., Mizuseki, K., Nishikawa, S., Kaneko, S., Kuwana, Y., Nakanishi, S., Nishikawa, S. I. and Sasai, Y. (2000) Induction of midbrain dopaminergic neurons from ES cells by stromal cell-derived inducing activity. Neuron, 28, 31-40. Gong, J., Sagiv, O., Cai, H., Tsang, S. H. and Del Priore, L. V. (2008) Effects of extracellular matrix and neighboring cells on induction of human embryonic stem cells into retinal or retinal pigment epithelial progenitors. Exp Eye Res, 86, 957-965.
Rust, W. L., Sadasivam, A. and Dunn, N. R. (2006) Three-dimensional extracellular matrix stimulates gastrulation-like events in human embryoid bodies. Stem Cells Dev, 15, 889-904.
Pereda, J., Zorn, T. and Soto-Suazo, M. (2006) Migration of human and mouse primordial germ cells and colonization of the developing ovary: an ultrastructural and cytochemical study. Microsc Res Tech, 69, 386-395.
Bendel-Stenzel, M., Anderson, R., Heasman, J. and Wylie, C. (1998) The origin and migration of primordial germ cells in the mouse. Semin Cell Dev Biol, 9, 393-400.
Mitalipova, M., Calhoun, J., Shin, S., Wininger, D., Schulz, T., Noggle, S., Venable, A., Lyons, I., Robins, A. and Stice, S. (2003) Human embryonic stem cell lines derived from discarded embryos. Stem Cells, 21, 521-526.
Gospodarowicz, D., Delgado, D. and Vlodaysky, I. (1980) Permissive effect of the extracellular matrix on cell proliferation in vitro. Proc Natl Acad Sci USA, 77, 4094-4098.
Rajaraman, S., Davis, W. S., Mahakali-Zama, A., Evans, H. K., Russell, L. B. and Bedell, M. A. (2002) An allelic series of mutations in the kit ligand gene of mice. I. Identification of point mutations in seven ethylnitrosourea-induced Kitl(Steel) alleles. Genetics, 162, 331-340.
Livak, K. J. and Schmittgen, T. D. (2001) Analysis of relative gene expression data using real-time quantitative PCR and the 2(-Delta Delta C(T)) Method. Methods, 25, 402-408.
Castrillon, D. H., Quade, B. J., Wang, T. Y., Quigley, C. and Crum, C. P. (2000) The human VASA gene is specifically expressed in the germ cell lineage. PNAS, 97, 9585-9590.
Toyooka, Y., Tsunekawa, N., Takahashi, Y., Matsui, Y., Satoh, M. and Noce, T. (2000) Expression and intracellular localization of mouse Vasa-homologue protein during germ cell development. Mech Dev, 93, 139-149.
Gaskell, T. L., Esnal, A., Robinson, L. L., Anderson, R. A. and Saunders, P. T. (2004) Immunohistochemical profiling of germ cells within the human fetal testis: identification of three subpopulations. Biol Reprod, 71, 2012-2021.
James Kehlerl, E. T., Birgit Koschorz2, Maurizio Pesce3, Luca Gentile4, Michele Boiani1, 8, Hilda Lomelí5, 6, Andras Nagy6, K John McLaughlin7, Hans R Schöler 1, 8 & Alexey Tomilin1, 2 (2004) Oct4 is required for primordial germ cell survival. EMBO, 5, 1078-1083.
Kerr, C. L., Hill, C. M., Blumenthal, P. D. and Gearhart, J. D. (2008) Expression of pluripotent stem cell markers in the human fetal testis. Stem Cells, 26, 412-421.
Ying, Y., Liu, X. M., Marble, A., Lawson, K. A. and Zhao, G. Q. (2000) Requirement of Bmp8b for the generation of primordial germ cells in the mouse. Mol Endocrinol, 14, 1053-1063.
Xu, R. H., Peck, R. M., Li, D. S., Feng, X., Ludwig, T. and Thomson, J. A. (2005) Basic FGF and suppression of BMP signaling sustain undifferentiated proliferation of human ES cells. Nat Methods, 2, 185-190.
Qi, X., Li, T. G., Hao, J., Hu, J., Wang, J., Simmons, H., Miura, S., Mishina, Y. and Zhao, G. Q. (2004) BMP4 supports self-renewal of embryonic stem cells by inhibiting mitogen-activated protein kinase pathways. Proc Natl Acad Sci USA, 101, 6027-6032.
Reik, W., Dean, W. and Walter, J. (2001) Epigenetic reprogramming in mammalian development. Science, 293, 1089-1093.
Schaefer, C. B., Ooi, S. K., Bestor, T. H. and Bourc'his, D. (2007) Epigenetic decisions in mammalian germ cells. Science, 316, 398-399.
Robinson, L. L. L., Gaskell, T. L., Saunders, P. T. K. and Anderson, R. A. (2001) Germ cell specific expression of c-kit in the human fetal gonad. Mol Hum Reprod, 7, 845-852.
Matsui, Y., Toksoz, D., Nishikawa, S., Nishikawa, S.-I., Williams, D., Zsebo, K. and Hogan, B. L. M. (1991) Effect of Steel factor and leukaemia inhibitory factor on murine primordial germ cells in culture. Nature, 353, 750-752.
Resnick, J. L., Bixler, L. S., Cheng, L. and Donovan, P. J. (1992) Long-term proliferation of mouse primordial germ cells in culture. Nature, 359, 550-551.
Pesce, M., Farrace, M. G., Piacentini, M., Dolci, S, and De Felici, M. (1993) Stem cell factor and leukemia inhibitory factor promote primordial germ cell survival by suppressing programmed cell death (apoptosis). Development, 118, 1089-1094.
Pesce, M., Di Carlo, A. and De Felici, M. (1997) The c-kit receptor is involved in the adhesion of mouse primordial germ cells to somatic cells in culture. Mech Dev, 68, 37-44.
Manova, K. and Bachvarova, R. F. (1991) Expression of c-kit encoded at the W locus of mice in developing embryonic germ cells and presumptive melanoblasts. Dev Biol, 146, 312-324.
Yuan, L., Liu, J. G., Hoja, M. R., Wilbertz, J., Nordqvist, K. and Hoog, C. (2002) Female germ cell aneuploidy and embryo death in mice lacking the meiosis-specific protein SCP3. Science, 296, 1115-1118.
Yuan, L., Liu, J. G., Zhao, J., Brundell, E., Daneholt, B. and Hoog, C. (2000) The murine SCP3 gene is required for synaptonemal complex assembly, chromosome synapsis, and male fertility. Mol Cell, 5, 73-83.
Chen, H. F., Kuo, H. C., Chien, C. L., Shun, C. T., Yao, Y. L., Ip, P. L., Chuang, C. Y., Wang, C. C., Yang, Y. S, and Ho, H. N. (2007) Derivation, characterization and differentiation of human embryonic stem cells: comparing serum-containing versus serum-free media and evidence of germ cell differentiation. Hum Reprod, 22, 567-577.
Clark, A. T., Bodnar, M. S., Fox, M., Rodriquez, R. T., Abeyta, M. J., Firpo, M. T. and Pera, R. A. (2004) Spontaneous differentiation of germ cells from human embryonic stem cells in vitro. Hum Mol Genet, 13, 727-739.
Kee, K., Gonsalves, J. M., Clark, A. T. and Pera, R. A. (2006) Bone morphogenetic proteins induce germ cell differentiation from human embryonic stem cells. Stem Cells Dev, 15, 831-837.
de Sousa Lopes, S. M. C., Roelen, B. A. J., Monteiro, R. M., Emmens, R., Lin, H. Y., Li, E., Lawson, K. A. and Mummery, C. L. (2004) BMP signaling mediated by ALK2 in the visceral endoderm is necessary for the generation of primordial germ cells in the mouse embryo. Genes Dev, 18, 1838-1849.

Skottman, H., Mikkola, M., Lundin, K., Olsson, C., Stromberg, A. M., Tuuri, T., Otonkoski, T., Hovatta, O. and Lahesmaa, R. (2005) Gene expression signatures of seven individual human embryonic stem cell lines. Stem Cells, 23, 1343-1356.
Rao, R. R., Calhoun, J. D., Qin, X., Rekaya, R., Clark, J. K. and Stice, S. L. (2004) Comparative transcriptional profiling of two human embryonic stem cell lines. Biotechnol Bioeng, 88, 273-286.
Kim, S. E., Kim, B. K., Gil, J. E., Kim, S. K. and Kim, J. H. (2007) Comparative analysis of the developmental competence of three human embryonic stem cell lines in vitro. Mol Cells, 23, 49-56.
Moore, J. C., Fu, J., Chan, Y. C., Lin, D., Tran, H., Tse, H. F. and Li, R. A. (2008) Distinct cardiogenic preferences of two human embryonic stem cell (hESC) lines are imprinted in their proteomes in the pluripotent state. Biochem Biophys Res Commun, 372, 553-558.
Monk, M., Boubelik, M. and Lehnert, S. (1987) Temporal and regional changes in DNA methylation in the embryonic, extraembryonic and germ cell lineages during mouse embryo development. Development, 99, 371-382.
Maatouk, D. M., Kellam, L. D., Mann, M. R., Lei, H., Li, E., Bartolomei, M. S. and Resnick, J. L. (2006) DNA methylation is a primary mechanism for silencing postmigratory primordial germ cell genes in both germ cell and somatic cell lineages. Development, 133, 3411-3418.
Tilgner, K., Atkinson, S. P., Golebiewska, A., Stojkovic, M., Lako, M. and Armstrong, L. (2008) Isolation of Primordial Germ Cells from Differentiating Human Embryonic Stem Cells. Stem Cells.

### REFERENCE COLLECTION 3

Eijpe, M., et al., Meiotic cohesin REC8 marks the axial elements of rat synaptonemal complexes before cohesins SMC1beta and SMC3. J Cell Biol, 2003. 160(5): p. 657-70.
2. Revenkova, E., et al., Novel meiosis-specific isoform of mammalian SMC1. Mol Cell Biol, 2001. 21(20): p. 6984-98.
3. Pelttari, J., et al., A meiotic chromosomal core consisting of cohesin complex proteins recruits DNA recombination proteins and promotes synapsis in the absence of an axial element in mammalian meiotic cells. Mol Cell Biol, 2001. 21(16): p. 5667-77.
4. Sun, F., et al., Meiotic studies in an azoospermic human translocation (Y;1) carrier. Mol Hum Reprod, 2005. 11(5): p. 361-4.
5. Topping, D., et al., Synaptic defects at meiosis I and non-obstructive azoospermia. Hum Reprod, 2006. 21(12): p. 3171-7.
6. Matzuk, M. M. and D. J. Lamb, The biology of infertility: research advances and clinical challenges. Nat Med, 2008. 14(11): p. 1197-213.
7. Uroz, L., O. Rajmil, and C. Templado, Premature separation of sister chromatids in human male meiosis. Hum Reprod, 2008. 23(4): p. 982-7.
8. Yuan, L., et al., The murine SCP3 gene is required for synaptonemal complex assembly, chromosome synapsis, and male fertility. Mol Cell, 2000. 5(1): p. 73-83.
9. Yuan, L., et al., Female germ cell aneuploidy and embryo death in mice lacking the meiosis-specific protein SCP3. Science, 2002. 296(5570): p. 1115-8.
10. Di Carlo, A. D., G. Travia, and M. De Felici, The meiotic specific synaptonemal complex protein SCP3 is expressed by female and male primordial germ cells of the mouse embryo. Int J Dev Biol, 2000. 44(2): p. 241-4.
11. Romanienko, P. J. and R. D. Camerini-Otero, The mouse Spo11 gene is required for meiotic chromosome synapsis. Mol Cell, 2000. 6(5): p. 975-87.
12. Rogakou, E. P., et al., DNA double-stranded breaks induce histone H2AX phosphorylation on serine 139. J Biol Chem, 1998. 273(10): p. 5858-68.
13. Bolor, H., et al., Mutations of the SYCP3 gene in women with recurrent pregnancy loss. Am J Hum Genet, 2009. 84(1): p. 14-20.
14. Leng, M., et al., Abnormal synapses and recombination in an azoospermic male carrier of a reciprocal translocation t(1;21). Fertil Steril, 2009. 91(4): p. 1293 e17-22.
15. Perrin, J., et al., Meiotic arrest at the midpachytene stage in a patient with complete azoospermia factor b deletion of the Y chromosome. Fertil Steril, 2006. 85(2): p. 494 e5-8.
16. Robinson, W. P., D. E. McFadden, and M. D. Stephenson, The origin of abnormalities in recurrent aneuploidy/polyploidy. Am J Hum Genet, 2001. 69(6): p. 1245-54.
17. Hassold, T. and P. Hunt, To err (meiotically) is human: the genesis of human aneuploidy. Nat Rev Genet, 2001. 2(4): p. 280-91.
18. Koubova, J., et al., Retinoic acid regulates sex-specific timing of meiotic initiation in mice. Proc Natl Acad Sci USA, 2006. 103(8): p. 2474-9.
19. Anderson, E. L., et al., Stra8 and its inducer, retinoic acid, regulate meiotic initiation in both spermatogenesis and oogenesis in mice. Proc Natl Acad Sci USA, 2008. 105(39): p. 14976-80.
20. Baltus, A. E., et al., In germ cells of mouse embryonic ovaries, the decision to enter meiosis precedes premeiotic DNA replication. Nat Genet, 2006. 38(12): p. 1430-4.
21. Bowles, J., et al., Retinoid signaling determines germ cell fate in mice. Science, 2006. 312(5773): p. 596-600.
22. Baarends, W. M. and J. A. Grootegoed, Chromatin dynamics in the male meiotic prophase. Cytogenet Genome Res, 2003. 103(3-4): p. 225-34.
23. West, F. D., et al., Enrichment and differentiation of human germ-like cells mediated by feeder cells and basic fibroblast growth factor signaling. Stem Cells, 2008. 26(11): p. 2768-76.
24. Bosch, P., S. L. Pratt, and S. L. Stice, Isolation, characterization, gene modification, and nuclear reprogramming of porcine mesenchymal stem cells. Biol Reprod, 2006. 74(1): p. 46-57.
25. Martin, R. H., Meiotic errors in human oogenesis and spermatogenesis. Reprod Biomed Online, 2008. 16(4): p. 523-31.
26. Codina-Pascual, M., et al., Synapsis and meiotic recombination analyses: MLH1 focus in the XY pair as an indicator. Hum. Reprod., 2005. 20(8): p. 2133-2139.
27. Antonarakis, S. E., et al., Chromosome 21 and down syndrome: from genomics to pathophysiology. Nat Rev Genet, 2004. 5(10): p. 725-38.
28. Lanfranco, F., et al., Klinefelter's syndrome. Lancet, 2004. 364(9430): p. 273-83.
29. Pfeiffer, B. E. and K. M. Huber, The State of Synapses in Fragile X Syndrome. Neuroscientist, 2009.
30. Mark, M., et al., STRA8-deficient spermatocytes initiate, but fail to complete, meiosis and undergo premature chromosome condensation. J Cell Sci, 2008. 121(Pt 19): p. 3233-42.
31. Menke, D. B., J. Koubova, and D.C. Page, Sexual differentiation of germ cells in XX mouse gonads occurs in an anterior-to-posterior wave. Dev Biol, 2003. 262(2): p. 303-12.
32. Bullejos, M. and P. Koopman, Germ cells enter meiosis in a rostro-caudal wave during development of the mouse ovary. Mol Reprod Dev, 2004. 68(4): p. 422-8.
33. Oulad-Abdelghani, M., et al., Characterization of a premeiotic germ cell-specific cytoplasmic protein encoded by Stra8, a novel retinoic acid-responsive gene. J Cell Biol, 1996. 135(2): p. 469-77.
34. Zhou, Q., et al., Expression of stimulated by retinoic acid gene 8 (Stra8) in spermatogenic cells induced by retinoic acid: an in vivo study in vitamin A-sufficient postnatal murine testes. Biol Reprod, 2008. 79(1): p. 35-42.
35. Lee, J., et al., Temporally and spatially selective loss of Rec8 protein from meiotic chromosomes during mammalian meiosis. J Cell Sci, 2003. 116(Pt 13): p. 2781-90.
36. Revenkova, E., et al., Cohesin SMC1 beta is required for meiotic chromosome dynamics, sister chromatid cohesion and DNA recombination. Nat Cell Biol, 2004. 6(6): p. 555-62.
37. Pittman, D. L., et al., Meiotic prophase arrest with failure of chromosome synapsis in mice deficient for Dmc1, a germline-specific RecA homolog. Mol Cell, 1998. 1(5): p. 697-705.
38. Yang, F., et al., Mouse TEX15 is essential for DNA double-strand break repair and chromosomal synapsis during male meiosis. J Cell Biol, 2008. 180(4): p. 673-9.
39. Hohenstein, K. A., et al., Nucleofection mediates high-efficiency stable gene knockdown and transgene expression in human embryonic stem cells. Stem Cells, 2008. 26(6): p. 1436-43.
40. Yates, F. and G. Q. Daley, Progress and prospects: gene transfer into embryonic stem cells. Gene Ther, 2006. 13(20): p. 1431-9.
41. Zwaka, T. P. and J. A. Thomson, Homologous recombination in human embryonic stem cells. Nat Biotechnol, 2003. 21(3): p. 319-21.
42. Eiges, R., et al., Establishment of human embryonic stem cell-transfected clones carrying a marker for undifferentiated cells. Curr Biol, 2001. 11(7): p. 514-8.
43. Ma, Y., et al., High-level sustained transgene expression in human embryonic stem cells using lentiviral vectors. Stem Cells, 2003. 21(1): p. 111-7.
44. Naldini, L., et al., In vivo gene delivery and stable transduction of nondividing cells by a lentiviral vector. Science, 1996. 272(5259): p. 263-7.
45. Chan, K. K., et al., Generation of high-level stable transgene expressing human embryonic stem cell lines using Chinese hamster elongation factor-1 alpha promoter system. Stem Cells Dev, 2008. 17(4): p. 825-36.
46. Hino, S., et al., Sea urchin insulator protects lentiviral vector from silencing by maintaining active chromatin structure. Gene Ther, 2004. 11(10): p. 819-28.
47. Vieyra, D. S, and M. A. Goodell, Pluripotentiality and conditional transgene regulation in human embryonic stem cells expressing insulated tetracycline-ON transactivator. Stem Cells, 2007. 25(10): p. 2559-66.
48. Toyooka, Y., et al., Embryonic stem cells can form germ cells in vitro. Proc Natl Acad Sci U S A, 2003. 100(20): p. 11457-62.
49. Hubner, K., et al., Derivation of Oocytes from Mouse Embryonic Stem Cells. Science, 2003. 300(5623): p. 1251-1256.
50. Geijsen, N., et al., Derivation of embryonic germ cells and male gametes from embryonic stem cells. Nature, 2004. 427(6970): p. 148-54.
51. Nayernia, K., et al., In vitro-differentiated embryonic stem cells give rise to male gametes that can generate offspring mice. Dev Cell, 2006. 11(1): p. 125-32.
52. Gomperts, M., et al., Interactions between primordial germ cells play a role in their migration in mouse embryos. Development, 1994. 120(1): p. 135-41.
53. Nayernia, K., et al., Derivation of male germ cells from bone marrow stem cells. Lab Invest, 2006. 86(7): p. 654-63.
54. Clark, A. T., et al., Spontaneous differentiation of germ cells from human embryonic stem cells in vitro. Hum Mol Genet, 2004. 13(7): p. 727-39.
55. Kee, K., et al., Bone morphogenetic proteins induce germ cell differentiation from human embryonic stem cells. Stem Cells Dev, 2006. 15(6): p. 831-7.
56. Tilgner, K., et al., Isolation of Primordial Germ Cells from Differentiating Human Embryonic Stem Cells. Stem Cells, 2008.
57. Bucay, N., et al., A Novel Approach for the Derivation of Putative Primordial Germ Cells and Sertoli Cells from Human Embryonic Stem Cells. Stem Cells, 2008.
58. Nishikawa, S., L. M. Jakt, and T. Era, Embryonic stem-cell culture as a tool for developmental cell biology. Nat Rev Mol Cell Biol, 2007. 8(6): p. 502-7.
59. Matsui, Y., et al., Effect of Steel factor and leukaemia inhibitory factor on murine primordial germ cells in culture. Nature, 1991. 353(6346): p. 750-752.
60. Resnick, J. L., et al., Long-term proliferation of mouse primordial germ cells in culture. Nature, 1992. 359(6395): p. 550-551.
61. Pesce, M., et al., Stem cell factor and leukemia inhibitory factor promote primordial germ cell survival by suppressing programmed cell death (apoptosis). Development, 1993. 118(4): p. 1089-94.
62. Resnick, J. L., et al., Role of Fibroblast Growth Factors and Their Receptors in Mouse Primordial Germ Cell Growth. Biol Reprod, 1998. 59(5): p. 1224-1229.
63. Lacham-Kaplan, O., H. Chy, and A. Trounson, Testicular cell conditioned medium supports differentiation of embryonic stem cells into ovarian structures containing oocytes. Stem Cells, 2006. 24(2): p. 266-73.
64. Shin, S., et al., Long-term proliferation of human embryonic stem cell-derived neuroepithelial cells using defined adherent culture conditions. Stem Cells, 2006. 24(1): p. 125-38.
65. Edelmann, W., et al., Meiotic pachytene arrest in MLH1-deficient mice. Cell, 1996. 85(7): p. 1125-34.
66. Barlow, A. L. and M. A. Hulten, Crossing over analysis at pachytene in man. Eur J Hum Genet, 1998. 6(4): p. 350-8.
67. Turnpenny, L., et al., Evaluating human embryonic germ cells: concord and conflict as pluripotent stem cells. Stem Cells, 2006. 24(2): p. 212-20.
68. Bosch, P., et al., Efficient adenoviral-mediated gene delivery into porcine mesenchymal stem cells. Mol Reprod Dev, 2006. 73(11): p. 1393-403.
69. Fouletier-Dilling, C. M., et al., Novel compound enables high-level adenovirus transduction in the absence of an adenovirus-specific receptor. Hum Gene Ther, 2005. 16(11): p. 1287-97.
70. Watanabe, K., et al., A ROCK inhibitor permits survival of dissociated human embryonic stem cells. Nat Biotechnol, 2007. 25(6): p. 681-6.
71. Pousette, A., et al., Presence of synaptonemal complex protein 1 transversal filament-like protein in human primary spermatocytes. Hum Reprod, 1997. 12(11): p. 2414-7.
72. Anderson, L. K., et al., Distribution of crossing over on mouse synaptonemal complexes using immunofluorescent localization of MLH1 protein. Genetics, 1999. 151(4): p. 1569-79.
73. Matsui, Y., K. Zsebo, and B. L. Hogan, Derivation of pluripotential embryonic stem cells from murine primordial germ cells in culture. Cell, 1992. 70(5): p. 841-7.
74. Shamblott, M. J., et al., Derivation of pluripotent stem cells from cultured human primordial germ cells. PNAS, 1998. 95(23): p. 13726-13731.
75. Kita, K., et al., Production of functional spermatids from mouse germline stem cells in ectopically reconstituted seminiferous tubules. Biol Reprod, 2007. 76(2): p. 211-7.
76. Honaramooz, A., et al., Building a testis: formation of functional testis tissue after transplantation of isolated porcine (Sus scrofa) testis cells. Biol Reprod, 2007. 76(1): p. 43-7.

1. A method of producing germ-like cells from pluripotent stem cells, the method comprising:
(a) differentiating pluripotent stem cells to germ-like cells in an adherent differentiation culture system comprising a fibroblast growth factor, wherein the cultured pluripotent stem cells are differentiated until about 60% to about 90%, or about 65% to about 85%, or about 70% to about 80% of the cells express one or more germ cell markers; and
(b) optionally isolating and collecting the germ-like cells.

2. The method according to claim 1 wherein said germ-like cells are capable of differentiating to haploid germ like cells that express sperm and egg specific genes.

3. The method of claim 1, wherein prior to differentiating, the embryonic stem cells are cultured in a culture medium comprising a basic fibroblast growth factor and the germ cell marker is DDX4 or POU5F1.

4. The method of claim 1, wherein the adherent differentiation culture system is either a feeder or feeder-free system.

5. The method of claim 1, wherein the adherent differentiation culture system comprises feeder cells.

6. The method of claim 5, wherein the feeder cells are selected from the group consisting of mouse embryonic fibroblast (MEF) feeder cells, feeder cells derived from human embryonic stem cells, feeder cells derived from the spontaneous differentiation of human embryonic stem cells, feeder cells obtained from human placenta, feeder cells derived from human foreskin, and feeder cells from human postnatal foreskin fibroblasts.

7. The method of claim 3, wherein the culture medium further comprises one or more component's selected from the group consisting of knockout serum replacement (KSR), a non-essential amino acid, an antiobiotic, and mercaptoethanol.

8. The method of claim 3, wherein during culturing the cells are passaged between 2 to 300, or between 50 to 250, or between 100 to 200 times.

9. The method of claim 1, wherein the pluripotent stem cells are human pluripotent stem cells and the fibroblast growth factor is basic fibroblast growth factor.

10. The method of claim 5, wherein:
(a) the embryonic stem cells are human embryonic stem cells and the feeder cells are mouse embryonic fibroblast feeder cells;
(b) prior to differentiation, the embryonic stem cells are cultured in a culture medium comprising basic fibroblast growth factor and one or more components selected from the group consisting of knockout serum replacement, a non-essential amino acid, an antiobiotic, and mercaptoethanol;
(c) prior to differentiation and during culturing, the cells are passaged between 1 to 200 times; and wherein
(d) the cultured embryonic stem cells are differentiated until about 65% to about 85% of the cells express DDX4 and POU5F1.

11. The method of claim 10, wherein after differentiation, between about 85% to about 95% of the cells express the meiotic markers SYCP3 and MLH1.

12. The method of claim 10, wherein the adherent differentiation culture system comprises basic fibroblast growth factor in a concentration of between about 2 ng/mL to about 10 ng/mL.

13. The method of claim 5, wherein the feeder cells are mouse embryonic fibroblast feeder cells that have been transformed to upregulate expression of KIT ligand.

14. The method of claim 5, wherein the feeder cells are mouse embryonic fibroblast feeder cells that have been transformed to upregulate expression of KIT ligand mRNA.

15. The method of claim 1, wherein the adherent differentiation culture system comprises a member of the TGF-β family.

16. The method of claim 15, wherein the adherent differentiation culture system comprises between about 10 ng/mL to about 150 ng/mL of BMP4.

17. The method of claim 1, wherein the cultured embryonic stem cells have been exposed to a member of the TGF-β family before differentiation.

18. The method of claim 1, wherein the cultured embryonic stem cells are differentiated to germ-like cells over a period of between about 3 to about 30 days.

19. The method of claim 18, wherein the cultured embryonic stem cells are differentiated to germ-like cells in about 5% CO2 and at a temperature of about 37° C.

20. The method of claim 1, wherein the adherent differentiation culture system comprises the extracellular matrix of fibroblast feeder cells.

21. The method of claim 20, wherein the extracellular matrix is the extracellular matrix of feeder cells are selected from the group consisting of mouse embryonic fibroblast feeder cells, feeder cells derived from human embryonic stem cells, feeder cells derived from the spontaneous differentiation of human embryonic stem cells, feeder cells obtained from human placenta, feeder cells derived from human foreskin, and feeder cells from human postnatal foreskin fibroblasts.

22. The method of claim 20, wherein the extracellular matrix is the extracellular matrix of mouse embryonic fibroblast feeder cells that have been transformed to upregulate the expression of either KIT ligand or KIT ligand mRNA.

23. The method of claim 22, wherein the adherent differentiation culture system comprises BMP4.

24. The method of claim 23, wherein the adherent differentiation culture system comprises between about 10 ng/mL to about 150 ng/mL of BMP4.

25. The method of claim 20, wherein the cultured embryonic stem cells are exposed to BMP4 before differentiation.

26. The method of claim 20, wherein the cultured embryonic stem cells are differentiated to germ-like cells over a period of between about 3 to about 30 days.

27. The method of claim 26, wherein the cultured embryonic stem cells are differentiated to germ-like cells in about 5% CO2 and at a temperature of about 37° C.

28. A method of producing a pure population of germ-like cells, the method comprising:
(a) differentiating cultured embryonic stem cells to germ-like cells in an adherent differentiation culture system comprising a fibroblast growth factor, wherein the cultured embryonic stem cells are differentiated until about 60% to about 90%, or about 65% to about 85%, or about 70% to about 80% of the cells express at least one germ cell marker;
(b) selecting individual differentiated cells that express at least one germ cell marker, propagating selected individual differentiated cells to form cell lines, and selecting for further differentiation those cell lines in which about 85% or more of the cells express at least one germ cell marker; and
(e) differentiating selected cells to meiotic germ-like cells in an adherent differentiation culture system.

29. The method according to claim 28 wherein said germ-like cells complete meiosis (haploid) and undergo specialization (sperm and oocyte specific markers).

30. The method of claim 28, wherein:
(a) the embryonic stem cells are human embryonic stem cells and the feeder cells are mouse embryonic fibroblast feeder cells;
(b) prior to differentiation, the embryonic stem cells are cultured in a culture medium comprising basic fibroblast growth factor and one or more components selected from the group consisting of knockout serum replacement, a non-essential amino acid, an antiobiotic, and mercaptoethanol;
(c) prior to differentiation in step (a) of claim 27 and during culturing, the embryonic stem cells are passaged (at least once, at least 10 times and preferably at least about 150 to 200 times);
(d) the cultured embryonic stem cells are differentiated until about 10+% (preferably about 85% or more) of the cells express DDX4 and POU5F1; and wherein
(e) the cell lines which are selected for further differentiation in step (b) of claim 27 are cell lines in which about 90% or more of the cells express DDX4 and POU5F1.

31. The method of claim 29, wherein:
(a) the embryonic stem cells are human embryonic stem cells and the feeder cells are mouse embryonic fibroblast feeder cells;
(b) prior to differentiation, the embryonic stem cells are cultured in a culture medium comprising basic fibroblast growth factor and one or more components selected from the group consisting of knockout serum replacement, a non-essential amino acid, an antiobiotic, and mercaptoethanol;
(c) prior to differentiation in step (a) of claim 27 and during culturing, the embryonic stem cells are passaged at least one time, preferably between about 150 to 200 times;
(d) the cultured embryonic stem cells are differentiated until the cells express DDX4 and POU5F1 at a level of at least about 10+% so as to be readily isolated (preferably at least about 85%); and wherein
(e) the cell lines which are selected for cloning in step (b) of claim 27 are cell lines in which (1) about 90% or more of the cells express DDX4 and POU5F1, and (2) about 70% or more of the cells express SYCP3 and MLH1.

32. A method of producing a pure population of germ-like cells, the method comprising:
(a) differentiating cultured embryonic stem cells to germ-like cells in an adherent differentiation culture system comprising a fibroblast growth factor, wherein the cultured embryonic stem cells are differentiated until about 60% to about 90%, or about 65% to about 85%, or about 70% to about 80% of the cells express at least one germ cell marker;
(b) selecting individual differentiated cells that express at least one germ cell marker, transducing selected individual differentiated cells with a reporter system containing STRA8, and propagating selected individual differentiated cells to form cell lines;
(c) selecting for further differentiation those cell lines which overexpress STRA8 and in which about 85% or more of the cells express at least one germ cell marker; and
(d) differentiating selected cells to meiotic germ-like cells in an adherent differentiation culture system.

33. The method of claim 32, wherein the reporter system is a viral vector comprising a first antibiotic resistance element under viral promoter control and a second antibiotic responsive-STA8 element under viral promoter control.

34. The method of claim 32, wherein:
(a) the embryonic stem cells are human embryonic stem cells and the feeder cells are mouse embryonic fibroblast feeder cells;
(b) prior to differentiation, the embryonic stem cells are cultured in a culture medium comprising basic fibroblast growth factor and one or more components selected from the group consisting of knockout serum replacement, a non-essential amino acid, an antiobiotic, and mercaptoethanol;
(c) prior to differentiation in step (a) of claim 30 and during culturing, the embryonic stem cells are passaged at least once and preferably between about 150 to 200 times;
(d) the cultured embryonic stem cells are differentiated until about 85% or more of the cells express DDX4 and POU5F1;
(e) the cell lines which are selected for further differentiation in step (b) of claim 27 are cell lines in which about 90% or more of the cells express DDX4 and POU5F1; and
(f) the reporter system is a viral vector comprising a first antibiotic resistance element under viral promoter control and a second antibiotic responsive-STA8 element under viral promoter control.

35. A method of producing germ-like cells from induced pluripotent stem cells, the method comprising:
(a) differentiating cultured induced pluripotent stem cells to germ-like cells in an adherent differentiation culture system comprising a fibroblast growth factor, wherein the cultured induced pluripotent stem cells are differentiated until about 60% to about 90%, or about 65% to about 85%, or about 70% to about 80% of the cells express one or more germ cell marker; and
(b) optionally isolating and collecting the germ-like cells.

36. The method of claim 35, wherein prior to differentiation the induced pluripotent stem cells are cultured in a culture medium comprising a fibroblast growth factor and the germ cell marker is DDX4 or POU5F1.

37. The method of claim 35, wherein the adherent differentiation culture system is either a feeder or feeder-free system.

38. The method of claim 35, wherein the adherent differentiation culture system comprises feeder cells.

39. The method of claim 38, wherein the feeder cells are selected from the group consisting of mouse embryonic fibroblast (MEF) feeder cells, feeder cells derived from human embryonic stem cells, feeder cells derived from the spontaneous differentiation of human embryonic stem cells, feeder cells obtained from human placenta, feeder cells derived from human foreskin, and feeder cells from human postnatal foreskin fibroblasts.

40. The method of claim 36, wherein the culture medium further comprises one or more components selected from the group consisting of knockout serum replacement (KSR), a non-essential amino acid, an antiobiotic, and mercaptoethanol.

41. The method of claim 36, wherein during culturing the cells are passaged between 2 to 300, or between 50 to 250, or between 100 to 200 times.

42. The method of claim 36, wherein the induced pluripotent stem cells are derived from human fibroblast cells and the fibroblast growth factor is basic fibroblast growth factor.

43. The method of claim 36, wherein:
(a) the induced pluripotent stem cells are derived from IMR90 lung fibroblast cells and the adherent differentiation culture system comprises mouse embryonic fibroblast feeder cells;
(b) prior to differentiation, the induced pluripotent stem cells are cultured in a culture medium comprising basic fibroblast growth factor and one or more components selected from the group consisting of knockout serum replacement, a non-essential amino acid, an antiobiotic, and mercaptoethanol;
(c) prior to differentiation and during culturing, the induced pluripotent stem cells are passaged between 150 to 200 times; and wherein
(d) the cultured induced pluripotent stem cells are differentiated until about 65% to about 85% of the cells express DDX4 and POU5F1.

44. The method of claim 43, wherein after differentiation, between about 85% to about 95% of the cells express the meiotic markers SYCP3 and MLH1.

45. The method of claim 43, wherein the adherent differentiation culture system comprises basic fibroblast growth factor in a concentration of between about 2 ng/mL to about 10 ng/mL.

46. The method of claim 38, wherein the feeder cells are mouse embryonic fibroblast feeder cells have been transformed to upregulate expression of KIT ligand.

47. The method of claim 38, wherein the feeder cells are mouse embryonic fibroblast feeder cells that have been transformed to upregulate expression of KIT ligand mRNA.

48. The method of claim 35, wherein the adherent differentiation culture system comprises a member of the TGF-β family.

49. The method of claim 48, wherein the adherent differentiation culture system comprises between about 10 ng/mL to about 150 ng/mL of BMP4.

50. The method of claim 35, wherein the induced pluripotent stem cells have been exposed to a member of the TGF-β family before differentiation.

51. The method of claim 35, wherein the cultured induced pluripotent stem cells are differentiated to germ-like cells over a period of between about 3 to about 30 days.

52. The method of claim 51, wherein the cultured induced pluripotent stem cells are differentiated to germ-like cells in about 5% CO2 and at a temperature of about 37° C.

53. The method of claim 35, wherein the adherent differentiation culture system comprises the extracellular matrix of fibroblast feeder cells.

54. The method of claim 53, wherein the extracellular matrix is the extracellular matrix of feeder cells are selected from the group consisting of mouse embryonic fibroblast feeder cells, feeder cells derived from human embryonic stem cells, feeder cells derived from the spontaneous differentiation of human embryonic stem cells, feeder cells obtained from human placenta, feeder cells derived from human foreskin, and feeder cells from human postnatal foreskin fibroblasts.

55. The method of claim 53, wherein the extracellular matrix is the extracellular matrix of mouse embryonic fibroblast feeder cells that have been transformed to upregulate the expression of either KIT ligand or KIT ligand mRNA.

56. The method of claim 55, wherein the adherent differentiation culture system comprises BMP4.

57. The method of claim 56, wherein the adherent differentiation culture system comprises between about 10 ng/mL to about 150 ng/mL of BMP4.

58. The method of claim 53, wherein the induced pluripotent stem cells are exposed to BMP4 before differentiation.

59. The method of claim 53, wherein the induced pluripotent stem cells are differentiated to germ-like cells over a period of between about 3 to about 30 days.

60. The method of claim 59, wherein the induced pluripotent stem cells are differentiated to germ-like cells in about 5% CO2 and at a temperature of about 37° C.

61. A method of producing a pure population of germ-like cells, the method comprising:
(a) differentiating cultured induced pluripotent stem cells to germ-like cells in an adherent differentiation culture system comprising a fibroblast growth factor, wherein the cultured induced pluripotent stem cells are differentiated until about 60% to about 90%, or about 65% to about 85%, or about 70% to about 80% of the cells express at least one germ cell marker;
(b) selecting individual differentiated cells that express at least one germ cell marker, propagating selected individual differentiated cells to form cell lines, and selecting for further differentiation those cell lines in which about 85% or more of the cells express at least one germ cell marker; and
(e) differentiating selected cells to meiotic germ-like cells in an adherent differentiation culture system.

62. The method of claim 61, wherein:
(a) the induced pluripotent stem cells are derived from IMR90 lung fibroblast cells and the feeder cells are mouse embryonic fibroblast feeder cells;
(b) prior to differentiation, the induced pluripotent stem cells are cultured in a culture medium comprising basic fibroblast growth factor and one or more components selected from the group consisting of knockout serum replacement, a non-essential amino acid, an antiobiotic, and mercaptoethanol;
(c) prior to differentiation in step (a) of claim 59 and during culturing, the induced pluripotent stem cells are passaged between 150 to 200 times;
(d) the cultured induced pluripotent stem cells are differentiated until about 85% or more of the cells express DDX4 and POU5F1; and wherein
(e) the cell lines which are selected for further differentiation in step (b) of claim 59 are cell lines in which about 90% or more of the cells express DDX4 and POU5F1.

63. The method of claim 59, wherein:
(a) the induced pluripotent stem cells are derived from IMR90 lung fibroblast cells and the feeder cells are mouse embryonic fibroblast feeder cells;
(b) prior to differentiation, the induced pluripotent stem cells are cultured in a culture medium comprising basic fibroblast growth factor and one or more components selected from the group consisting of knockout serum replacement, a non-essential amino acid, an antiobiotic, and mercaptoethanol;
(c) prior to differentiation in step (a) of claim 59 and during culturing, the induced pluripotent stem cells are passaged at least once and preferably, between about 150 to 200 times;
(d) the cultured induced pluripotent stem cells are differentiated until about 10+% (preferably about 85%) or more of the cells express DDX4 and POU5F1; and wherein
(e) the cell lines which are selected for cloning in step (b) of claim 59 are cell lines in which (1) about 90% or more of the cells express DDX4 and POU5F1, and (2) about 70% or more of the cells express SYCP3 and MLH1.

64. A method of producing a pure population of germ-like cells, the method comprising:
(a) differentiating induced pluripotent stem cells to germ-like cells in an adherent differentiation culture system comprising a fibroblast growth factor, wherein the cultured induced pluripotent stem, cells are differentiated until about 60% to about 90%, or about 65% to about 85%, or about 70% to about 80% of the cells express at least one germ cell marker;
(b) selecting individual differentiated cells that express at least one germ cell marker, transducing selected individual differentiated cells with a reporter system containing STRA8 and propagating selected individual differentiated cells to form cell lines;
(c) selecting for further differentiation those cell lines which overexpress STRA8 and in which about 85% or more of the cells express at least one germ cell marker; and
(d) differentiating selected cells to meiotic germ-like cells in an adherent differentiation culture system.

65. The method of claim 64, wherein the reporter system is a viral vector comprising a first antibiotic resistance element under viral promoter control and a second antibiotic responsive-STA8 element under viral promoter control.

66. The method of claim 64, wherein:
(a) the induced pluripotent stem cells are derived from IMR90 lung fibroblast cells and the feeder cells are mouse embryonic fibroblast feeder cells;
(b) prior to differentiation, the induced pluripotent stem cells are cultured in a culture medium comprising basic fibroblast growth factor and one or more components selected from the group consisting of knockout serum replacement, a non-essential amino acid, an antiobiotic, and mercaptoethanol;
(c) prior to differentiation in step (a) of claim 62 and during culturing, the induced pluripotent stem cells are passaged between 150 to 200 times;
(d) the cultured induced pluripotent stem cells are differentiated until about 85% or more of the cells express DDX4 and POU5F1;
(e) the cell lines which are selected for further differentiation in step (b) of claim 62 are cell lines in which about 90% or more of the cells express DDX4 and POU5F1; and
(f) the reporter system is a viral vector comprising a first antibiotic resistance element under viral promoter control and a second antibiotic responsive-STA8 element under viral promoter control.

66. A germ-like cell made by a method of any of claims 1-64.

67-81. (canceled)

82. A pharmaceutical composition comprising a germ-like cell made by a method of claim 1 and a pharmaceutically acceptable excipient or carrier.

83. The composition according to claim 82 wherein said germ-like cell is cryopreserved.

84. A kit comprising a germ-like cell made by a method of claim 1 optionally in combination with a cell culture medium.

85. (canceled)


SEQUENCE LISTING (2 sequences)

SEQ ID NO: 1 - DNA, 20 bp, Artificial Sequence
  sense strand primer for amplification of Cyp26b1
tctttgagggcttggatctg

SEQ ID NO: 2 - DNA, 19 bp, Artificial Sequence
  antisense strand primer for amplification of Cyp26b1
gaattggacaccgtgttgg